(19) United States
(12) Patent Application Publication
(10) Pub. No.: US 2024/0066099 A1
(43) Pub. Date: Feb. 29, 2024

(54) BDNF OTIC FORMULATIONS AND USE THEREOF

(71) Applicant: DOMPÉ FARMACEUTICI S.P.A., Milan (IT)

(72) Inventor: Jeff Anderson, San Diego, CA (US)

(21) Appl. No.: 18/267,739

(22) PCT Filed: Dec. 15, 2021
(86) PCT No.: PCT/US2021/010053

§ 371 Date: Jun. 15, 2023

Related U.S. Application Data
(60) Provisional application No. 63/125,902, filed on Dec. 15, 2020.

(51) Int. Cl. A61K 38/18; A61K 9/00; A61K 47/10; A61P 27/16

(52) U.S. Cl. CPC A61K 38/185; A61K 9/0019; A61K 9/0046; A61K 47/10; A61P 27/16

(57) ABSTRACT

Disclosed herein are otic formulations and compositions comprising BDNF. These otic formulations and compositions allow for the delivery of BDNF to the inner ear for the treatment of otic diseases and disorders.

### BACKGROUND OF THE DISCLOSURE

Vertebrates have a pair of ears, placed symmetrically on opposite sides of the head. The ear serves as both the sense organ that detects sound and the organ that maintains balance and body position. The ear is generally divided into three portions: the outer ear, auris media (or middle ear), and the auris interna (or inner ear).

### SUMMARY OF THE DISCLOSURE

Provided herein in some embodiments is a method of treating hearing loss or hearing impairement in a human subject, comprising intratympanically administering an otic formulation to the human subject, wherein the otic composition comprises from about 0.005 mg to about 0.40 mg of brain-derived neurotrophic factor (BDNF) and an auris-acceptable vehicle, wherein the otic formulation is formulated to provide sustained release of BDNF into the inner ear.

In some embodiments, the BDNF is a recombinant BDNF.

In some embodiments, the composition comprises from 0.005 mg to 0.015 mg BDNF. In some embodiments, the composition comprises about 0.01 mg BDNF.

In some embodiments, the composition comprises from 0.015 mg to 0.05 mg BDNF. In some embodiments, the composition comprises about 0.03 mg BDNF.

. In some embodiments, the composition comprises from 0.05 mg to 0.20 mg BDNF. In some embodiments, the composition comprises about 0.10 mg BDNF.

In some embodiments, the composition comprises from 0.20 mg to 0.40 mg BDNF. In some embodiments, the composition comprises about 0.3 mg BDNF.

In some embodiments, the composition comprises from 0.40 mg to 1.10 mg BDNF. In some embodiments, the composition comprises about 0.75 mg or about 0.78 mg BDNF.

In some embodiments, the composition comprises from 1.10 mg to 1.90 mg BDNF. In some embodiments, the composition comprises about 1.5 mg or about 1.56 mg BDNF.

In some embodiments, the auris-acceptable vehicle is an auris-acceptable gel.

In some embodiments, the auris-acceptable gel is a thermoreversible gel.

In some embodiments, the auris-acceptable gel comprises a copolymer of polyoxyethylene and polyoxypropylene.

In some embodiments, the copolymer of polyoxyethylene and polyoxypropylene is poloxamer 407.

In some embodiments, the otic formulation comprises from about 14 wt % to about 18 wt % poloxamer 407.

In some embodiments, the otic formulation comprises from about 15 wt % to about 17 wt % poloxamer 407.

In some embodiments, the otic formulation comprises about 15.8 wt % or about 16 wt % poloxamer 407.

In some embodiments, the auris-acceptable gel has a gelation viscosity from about 15,000 cP and about 3,000,000 cP.

In some embodiments, the auris-acceptable gel is capable of being injected by a narrow gauge needle or cannula through the tympanic membrane.

In some embodiments, the otic formulation has an osmolarity from about 100 mOsm/L to about 1000 mOsm/L.

In some embodiments, the otic formulation has a gelation temperature from about 19° C. to about 42° C.

In some embodiments, the otic formulation has a pH from about 7.0 to about 8.0.

In some embodiments, the otic formulation is an aqueous formulation and essentially free of any non-aqueous solvents.

In some embodiments, the growth factor is dissolved in the otic formulation.

In some embodiments, the growth factor is suspended in the otic formulation.

In some embodiments, the otic formulation provides sustained release of BDNF into the inner ear over a period of at least 5 days.

In some embodiments, the otic formulation provides sustained release of BDNF into the inner ear over a period of at least 1 week.

In some embodiments, the otic formulation provides sustained release of BDNF into the inner ear over a period of at least 2 weeks.

In some embodiments, the otic formulation provides sustained release of BDNF into the inner ear over a period of at least 3 weeks.

In some embodiments, the otic formulation provides sustained release of BDNF into the inner ear over a period of at least 4 weeks.

In some embodiments, the otic formulation repairs ribbon synapses.

In some embodiments, the hearing loss or hearing impairement is selected from cochlear synaptopathy, hearing-in-noise difficulties, speech-in-noise hearing impairement, or combinations thereof.

In some embodiments, the hearing loss or hearing impairement is cochlear synaptopathy.

In some embodiments, the hearing loss or hearing impairement is hearing-in-noise difficulties.

In some embodiments, the hearing loss or hearing impairement is speech-in-noise hearing impairement.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates the anatomy of the ear;

FIG. 2 illustrates inner ear PK of COMPOSITION A following intratympanic injection in rats;

FIG. 3 illustrates hearing function following a single intratympanic administration of COMPOSITION A in Rats (2-week Recovery Cohort). ABR threshold shifts (4, 10 and 20 kHz) in the treated ear at termination (2-weeks) from baseline values are reported as mean±SEM for male and female rats. Treatment groups were: saline (black), gentamicin (red), P407 vehicle (purple), 0.05% COMPOSITION A (light blue), 0.15% COMPOSITION A (blue) and 0.5% COMPOSITION A (dark blue);

FIG. 4 illustrates hearing function following a single intratympanic administration of COMPOSITIOTN A in Rats (3-month Recovery Cohort). ABR threshold shifts (4, 10 and 20 kHz) in the treated ear at termination (3-month) from baseline values are reported as mean±SEM for male and female rats. Treatment groups were: saline (black), gentamicin (red), P407 vehicle (purple), 0.05% COMPOSITION A (light blue), 0.15% COMPOSITION A (blue) and 0.5% COMPOSITION A (dark blue);

FIG. 5 illustrates hearing function following a single intratympanic administration of COMPOSITION A in Cats (2-Week Recovery Period). ABR threshold shifts (4, 10 and 20 kHz) in the treated ear at termination from baseline values are reported as mean±SEM for male and female cats. Treatment groups were: saline (black), gentamicin (red), P407 vehicle (purple), 0.05% COMPOSITION A (light blue), 0.15% COMPOSITION A (blue) and 0.5% COMPOSITION A (dark blue);

FIG. 6 illustrates hearing function following a single intratympanic administration of COMPOSITION A in Cats (3-Month Recovery Period). ABR threshold shifts (4, 10 and 20 kHz) in the treated ear at termination (3-month) from baseline values are reported as mean±SEM for male and female cats. Treatment groups were: saline (black), gentamicin (red), P407 vehicle (purple), 0.05% COMPOSITION A (light blue), 0.15% COMPOSITION A (blue) and 0.5% COMPOSITION A (dark blue);

FIG. 7 illustrates Digits in Noise test (DIN) speech reception threshold scores for individual subjects at testing time 1 (DIN 1) and testing time 2 (DIN 2) approximately 1 week apart. DIN SRT scores (db SNR) were strongly correlated indicating good test/re-test reliability;

FIG. 8 illustrate non-limiting examples of speech-in-noise tests used in certain embodiments disclosed herein;

FIG. 9. illustrates safty analysis of COMPOSITION A in a clinical study, showing subject disposition (top), overall adverse events (middle), and ear related advere events (bottom);

FIG. 10. illustrates the overall clinical efficacy of a clinical study of COMPOSITION A;

FIG. 11. illustrates the clinical efficacy for s subset of the patients with moderate-to-severe hearing loss in a clinical study of COMPOSITION A; and

FIG. 12. illustrates correlation between improvement in SIN tests and improvement in speech intelligibility.

### DETAILED DESCRIPTION

Systemic administration of active agents is, in some instances, ineffectual in the treatment of diseases that affect inner ear structures. The cochlear canals and the cochlea, for example, are isolated from the circulatory system limiting systemic delivery of active agents to target sites in the inner ear. In some instances, systemic drug administration creates a potential inequality in drug concentration with higher circulating levels in the serum, and lower levels in the target auris interna organ structures. In certain instances, large amounts of drug are required to overcome this inequality in order to deliver sufficient, therapeutically effective quantities of a drug to auditory structures. In some instances, systemic drug administration also increases the likelihood of secondary systemic accumulation and consequent adverse side effects.

Currently available treatment for inner ear diseases also carries the risk of attendant side effects. For example, available methods require multiple daily doses (e.g., intratympanic injection or infusion) of drugs. In certain instances, multiple daily intratympanic injections cause patient discomfort and non-compliance. In certain instances, delivery of active agents to the inner ear via otic drops administered in the ear canal or via intratympanic injection is hindered by the biological barrier presented by the tympanic membrane the oval window membrane and/or the round window membrane. In some instances, delivery of active agents to the inner ear via otic drops or intratympanic injection causes osmotic imbalance in inner ear structures, introduces infections or other immune disorders as a result of microbial or endotoxin presence, or results in permanent structural damage (e.g. perforation of the tympanic membrane), resulting in hearing loss and the like.

Intratympanic injection of therapeutic agents is the technique of injecting a therapeutic agent behind the tympanic membrane into the auris media and/or auris interna. Some challenges remain with intratympanic injections. For example, access to the round window membrane, the site of drug absorption into the auris interna, is challenging in some instances. In addition, current regimens using intratympanic injections do not address changing the osmolarity and pH of the peri lymph and endolymph, and introducing pathogens and endotoxins that directly or indirectly damage inner ear.

Provided herein in one aspect are otic formulations and compositions comprising a therapeutically effective amount of brain-derived neurotrophic factor (BDNF). In some embodiments, the otic formulations are auris-acceptable gels. In some embodiments, the otic formulations are triglyceride based auris-acceptable formulations.

These otic pharmaceutical formulations are suitable for drug delivery into the external, middle and/or inner ear. In some instances, these otic pharmaceutical formulations and compositions are suitable for administration to humans. In some instances, the otic formulations and compositions disclosed herein also meet stringent criteria for pH, osmolarity, ionic balance, sterility, endotoxin, and/or pyrogen levels. In some instances, the otic formulations and compositions are compatible with the microenvironment of the inner ear (e.g., the perilymph).

Accordingly, provided herein, in certain embodiments, are otic formulations and compositions that are controlled release auris-acceptable formulations and compositions that locally treat auris target structures and provide extended exposure of otic active agents to the target auris structures. In certain embodiments, the otic formulations and compositions described herein are designed for stringent osmolarity and pH ranges that are compatible with auditory structures and/or the endolymph and perilymph. In some embodiments, the otic formulations and compositions described herein are controlled release formulations that provide extended release for a period of at least 3 days and meet stringent sterility requirements. In some instances, otic formulations and compositions described herein contain lower endotoxin levels (e.g., <0.5 EU/mL when compared to typically acceptable endotoxin levels of 0.5 EU/mL. In some instances, the otic formulations and compositions described herein contain low levels of colony forming units (e.g., <50 CFUs) per gram of the formulation or composition. In some instances, the otic formulations or compositions described herein are substantially free of pyrogens and/or microbes. In some instances, the otic formulations or compositions described herein are formulated to preserve the ionic balance of the endolymph and/or the perilymph.

In some instances, local administration of the otic formulations and compositions described herein avoids potential adverse side effects as a result of systemic administration of active agents. In some instances, the locally applied otic formulations and compositions described herein are compatible with auris structures. Such compatible auris structures include those associated with the outer, middle, and/or inner ear. In some embodiments, the otic formulations and compositions are administered either directly to the desired auris structure, e.g. the cochlear region, or administered to a structure in direct communication with areas of the auris structure; in the case of the cochlear region, for example, including but not limited to the round window membrane, the crista fenestrae cochleae or the oval window membrane.

In certain instances, the otic formulations and compositions disclosed herein controlled release formulations or compositions that provide a constant rate of release of a drug from the formulation and provide a constant prolonged source of exposure of an otic active agent to the inner ear of an individual or patient suffering from an otic disorder, reducing or eliminating any variabilities associated with other methods of treatment (such as, e.g., otic drops and/or multiple intratympanic injections).

In some embodiments, the otic formulations and compositions described herein provide extended release of the active ingredient(s) into the external ear. In some embodiments, the otic formulations and compositions described herein provide extended release of the active ingredient(s) into the middle and/or inner ear (auris interna), including the cochlea and vestibular labyrinth. In some embodiments, the otic formulations and compositions further comprise an immediate or rapid release component in combination with a controlled release component. Certain Definitions

The term “auris-acceptable” with respect to a formulation, composition or ingredient, as used herein, includes having no persistent detrimental effect on the auris externa (or external ear or outer ear), auris media (or middle ear) and/or the auris interna (or inner ear) of the subject being treated. By “auris-pharmaceutically acceptable,” as used herein, refers to a material, such as a carrier or diluent, which does not abrogate the biological activity or properties of the compound in reference to the auris externa (or external ear or outer ear), auris media (or middle ear) and/or the auris interna (or inner ear), and is relatively or is reduced in toxicity to the auris externa (or external ear or outer ear), auris media (or middle ear) and the auris interna (or inner ear), i.e., the material is administered to an individual without causing undesirable biological effects or interacting in a deleterious manner with any of the components of the composition in which it is contained.

As used herein, amelioration or lessening of the symptoms of a particular otic disease, disorder or condition by administration of a particular compound or pharmaceutical composition refers to any decrease of severity, delay in onset, slowing of progression, or shortening of duration, whether permanent or temporary, lasting or transient that is attributed to or associated with administration of the compound or composition.

“Auris externa” refers to the external (or outer) ear, and includes the pinna and the external auditory canal (EAC).

“Auris interna” refers to the inner ear, including the cochlea and the vestibular labyrinth, and the round window that connects the cochlea with the middle ear.

. “Auris-interna bioavailability” or “Auris media bioavailability” refers to the percentage of the administered dose of compounds disclosed herein that becomes available in the inner or middle ear, respectively, of the animal or human being studied.

“Auris media” refers to the middle ear, including the tympanic cavity, auditory ossicles and oval window, which connects the middle ear with the inner ear.

“Auris-interna bioavailability” refers to the percentage of the administered dose of compounds disclosed herein that becomes available in the inner ear of the animal or human being studied.

“Balance disorder” refers to a disorder, illness, or condition which causes a subject to feel unsteady, or to have a sensation of movement. Included in this definition are dizziness, vertigo, disequilibrium, and pre-syncope. Diseases which are classified as balance disorders include, but are not limited to, Ramsay Hunt's Syndrome, Meniere's Disease, mal de debarquement, benign paroxysmal positional vertigo, labyrinthitis, and presbycusis.

“Blood plasma concentration” refers to the concentration of compounds provided herein in the plasma component of blood of a subject.

“Carrier materials” are excipients that are compatible with the otic agent, the auris media, the auris interna and the release profile properties of the auris-acceptable pharmaceutical formulations. Such carrier materials include, e.g., binders, suspending agents, disintegration agents, filling agents, surfactants, solubilizers, stabilizers, lubricants, wetting agents, diluents, and the like. “Auris-pharmaceutically compatible carrier materials” include, but are not limited to, acacia, gelatin, colloidal silicon dioxide, calcium glycerophosphate, calcium lactate, maltodextrin, glycerine, magnesium silicate, polyvinylpyrrolidone (PVP), cholesterol, cholesterol esters, sodium caseinate, soy lecithin, taurocholic acid, phosphatidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, alginate, carbomer, hyaluronic acid (HA), poloxamer, dextran, and the like.

As used herein, the term “cytotoxic agent” refers to compounds that are cytotoxic (i.e., toxic to a cell) effective for the treatment of otic disorders, e.g., autoimmune diseases of the ear and cancer of the ear, and are suitable for use in the formulations disclosed herein.

The term “diluent” are chemical compounds that are used to dilute the otic agent prior to delivery and which are compatible with the auris media and/or auris interna.

“Drug absorption” or “absorption” refers to the process of movement of the otic agent from the localized site of administration, by way of example only, the round window membrane of the inner ear, and across a barrier (the round window membranes, as described below) into the auris interna or inner ear structures. The terms “co-administration” or the like, as used herein, are meant to encompass administration of the otic agent to a single patient, and are intended to include treatment regimens in which the otic agents are administered by the same or different route of administration or at the same or different time.

The terms “effective amount” or “therapeutically effective amount,” as used herein, refer to a sufficient amount of the otic agent being administered that would be expected to relieve to some extent one or more of the symptoms of the disease or condition being treated. For example, the result of administration of the otic agents disclosed herein is reduction and/or alleviation of the signs, symptoms, or causes of any one of the diseases or conditions disclosed herein. For example, an “effective amount” for therapeutic uses is the amount of the otic agent, including a formulation as disclosed herein required to provide a decrease or amelioration in disease symptoms without undue adverse side effects. The term “therapeutically effective amount” includes, for example, a prophylactically effective amount. An “effective amount” of a otic agent composition disclosed herein is an amount effective to achieve a desired pharmacologic effect or therapeutic improvement without undue adverse side effects. It is understood that “an effective amount” or “a therapeutically effective amount” varies, in some embodiments, from subject to subject, due to variation in metabolism of the compound administered, age, weight, general condition of the subject, the condition being treated, the severity of the condition being treated, and the judgment of the prescribing physician. In some instances, it is also understood that “an effective amount” in an extended-release dosing format differs from “an effective amount” in an immediate-release dosing format based upon pharmacokinetic and pharmacodynamic considerations.

The terms “enhance” or “enhancing” refers to an increase or prolongation of either the potency or duration of a desired effect of the otic agent, or a diminution of any adverse symptomatology. For example, in reference to enhancing the effect of the otic agents disclosed herein, the term “enhancing” refers to the ability to increase or prolong, either in potency or duration, the effect of other therapeutic agents that are used in combination with the otic agents disclosed herein. An “enhancing-effective amount,” as used herein, refers to an amount of an otic agent or other therapeutic agent that is adequate to enhance the effect of another therapeutic agent or otic agent in a desired system. When used in a patient, amounts effective for this use will depend on the severity and course of the disease, disorder or condition, previous therapy, the patient's health status and response to the drugs, and the judgment of the treating physician.

The term “inhibiting” includes preventing, slowing, or reversing the development of a condition, including any of one of the conditions described herein, or advancement of a condition in a patient necessitating treatment.

The terms “kit” and “article of manufacture” are used as synonyms.

“Local anesthetic” means a substance which causes a reversible loss of sensation and/or a loss of nociception. Often, these substances function by decreasing the rate of the depolarization and repolarization of excitable membranes (for example, neurons). By way of non-limiting example, local anesthetics include lidocaine, benzocaine, prilocaine, and tetracaine.

As used herein, the term “otic agent” or “otic structure modulating agent” or “otic therapeutic agent” or “otic active agent” or “active agent” or “therapeutic agent” refers to compounds that are effective for the treatment of otic disorders, e.g., otitis media, otosclerosis, autoimmune diseases of the ear and cancer of the ear, and are suitable for use in the formulations disclosed herein. An “otic agent” or “otic structure modulating agent” or “otic therapeutic agent” or “otic active agent” or “active agent” includes, but is not limited to, compounds that act as an agonist, a partial agonist, an antagonist, a partial antagonist, an inverse agonist, a competitive antagonist, a neutral antagonist, an orthosteric antagonist, an allosteric antagonist, a positive allosteric modulator of an otic structure modulating target, a negative allosteric modulator of an otic structure modulating target, or combinations thereof.

The term “otic intervention” means an external insult or trauma to one or more auris structures and includes implants, otic surgery, injections, cannulations, or the like. Implants include auris-interna or auris-media medical devices, examples of which include cochlear implants, hearing sparing devices, hearing-improvement devices, short electrodes, micro-prostheses or piston-like prostheses; needles; stem cell transplants; drug delivery devices; any cell-based therapeutic; or the like. Otic surgery includes middle ear surgery, inner ear surgery, tympanostomy, cochleostomy, labyrinthotomy, mastoidectomy, stapedectomy, stapedotomy, endolymphatic sacculotomy, or the like. Injections include intratympanic injections, intracochlear injections, injections across the round window membrane or the like. Cannulations include intratympanic, intracochlear, endolymphatic, perilymphatic or vestibular cannulations, or the like.

“Pharmacokinetics” refers to the factors which determine the attainment and maintenance of the appropriate concentration of drug at the desired site within the auris media and/or auris interna.

In prophylactic applications, compositions containing the otic agents described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like.

As used herein, the term “subject” is used to mean an animal, preferably a mammal, including a human or non-human. The terms patient and subject are used interchangeably.

The terms “treat,” “treating” or “treatment,” as used herein, include alleviating, abating or ameliorating a disease or condition or the associated symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or controlling or stopping the symptoms of the disease or condition either prophylactically and/or therapeutically.

The term “substantially low degradation products” means about 10% by weight of the active agent are degradation products of the active agent. In further embodiments, the term means less than 10% by weight of the active agent are degradation products of the active agent. In further embodiments, the term means less than 9% by weight of the active agent are degradation products of the active agent. In further embodiments, the term means less than 8% by weight of the active agent are degradation products of the active agent. In further embodiments, the term means less than 7% by weight of the active agent are degradation products of the active agent. In further embodiments, the term means less than 6% by weight of the active agent are degradation products of the active agent. In further embodiments, the term means less than 5% by weight of the active agent are degradation products of the active agent. In further embodiments, the term means less than 4% by weight of the active agent are degradation products of the active agent. In further embodiments, the term means less than 3% by weight of the active agent are degradation products of the active agent. In yet further embodiments, the term means less than 2% by weight of the active agent are degradation products of the active agent. In further embodiments, the term means less than 1% by weight of the active agent are degradation products of the active agent. In some embodiments, any individual impurity (e.g., metal impurity, degradation products of active agent and/or excipients, or the like) present in a formulation described herein is less than 5%, less than 2%, or less than 1% by weight of the active agent. In some embodiments the formulation does not contain precipitate during storage or change in color after manufacturing and storage.

Other objects, features, and advantages of the methods and compositions described herein will become apparent from the following detailed description. It should be understood, however, that the detailed description and the specific examples, while indicating specific embodiments, are given by way of illustration only.

### Anatomy of the Ear

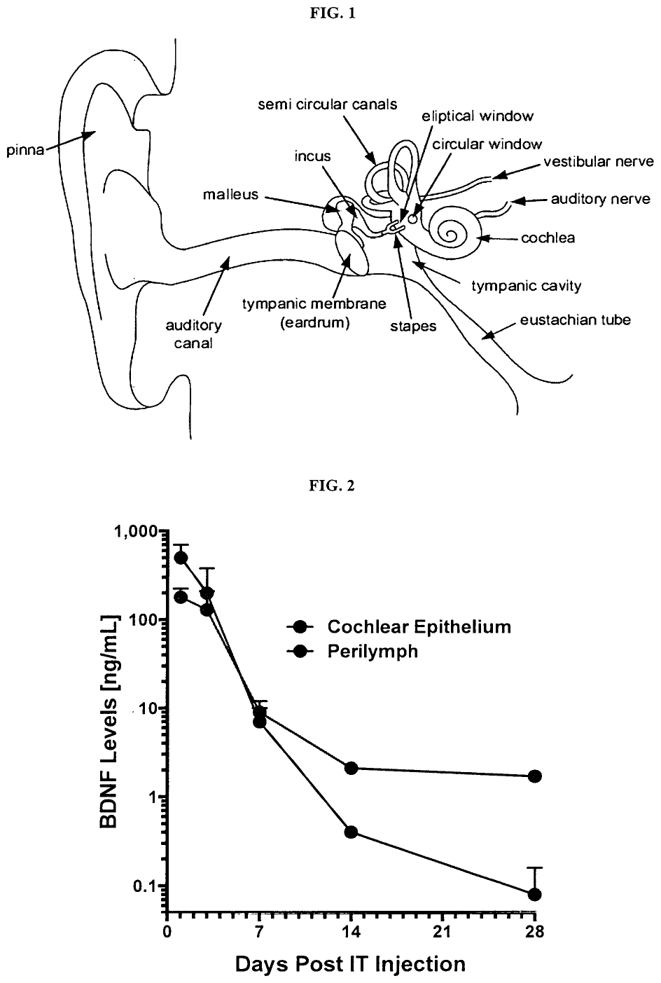

The ear serves as both the sense organ that detects sound and the organ that maintains balance and body position. The ear is generally divided into three portions: the outer ear, middle ear and the inner ear (or auris interna). As shown in FIG. 1, the outer ear is the external portion of the organ and is composed of the pinna (auricle), the auditory canal (external auditory meatus) and the outward facing portion of the tympanic membrane, also known as the ear drum. The pinna, which is the fleshy part of the externa ear that is visible on the side of the head, collects sound waves and directs them toward the auditory canal. Thus, the function of the outer ear, in part, is to collect and direct sound waves towards the tympanic membrane and the middle ear.

The middle ear is an air-filled cavity, called the tympanic cavity, behind the tympanic membrane. The tympanic membrane, also known as the ear drum, is a thin membrane that separates the external ear from the middle ear. The middle ear lies within the temporal bone, and includes within this space the three ear bones (auditory ossicles): the malleus, the incus and the stapes. The auditory ossicles are linked together via tiny ligaments, which form a bridge across the space of the tympanic cavity. The malleus, which is attached to the tympanic membrane at one end, is linked to the incus at its anterior end, which in turn is linked to the stapes. The stapes is attached to the oval window, one of two windows located within the tympanic cavity. A fibrous tissue layer, known as the annular ligament connects the stapes to the oval window. Sound waves from the outer ear first cause the tympanic membrane to vibrate. The vibration is transmitted across to the cochlea through the auditory ossicles and oval window, which transfers the motion to the fluids in the auris interna. Thus, the auditory ossicles are arranged to provide a mechanical linkage between the tympanic membrane and the oval window of the fluid-filled auris interna, where sound is transformed and transduced to the auris interna for further processing. Stiffness, rigidity or loss of movement of the auditory ossicles, tympanic membrane or oval window leads to hearing loss, e.g. otosclerosis, or rigidity of the stapes bone.

The tympanic cavity also connects to the throat via the eustachian tube. The eustachian tube provides the ability to equalize the pressure between the outside air and the middle ear cavity. The round window, a component of the auris interna but which is also accessible within the tympanic cavity, opens into the cochlea of the auris interna. The round window is covered by a membrane, which consists of three layers: an external or mucous layer, an intermediate or fibrous layer, and an internal membrane, which communicates directly with the cochlear fluid. The round window, therefore, has direct communication with the auris interna via the internal membrane.

Movements in the oval and round window are interconnected, i.e. as the stapes bone transmits movement from the tympanic membrane to the oval window to move inward against the auris interna fluid, the round window is correspondingly pushed out and away from the cochlear fluid. This movement of the round window allows movement of fluid within the cochlea, which eventually leads in turn to movement of the cochlear inner hair cells, allowing hearing signals to be transduced: Stiffness and rigidity in the round window leads to hearing loss because of the lack of ability of movement in the cochlear fluid. Recent studies have focused on implanting mechanical transducers onto the round window, which bypasses the normal conductive pathway through the oval window and provides amplified input into the cochlear chamber.

Auditory signal transduction takes place in the auris interna. The fluid-filled inner ear, or auris interna; consists of two major components: the cochlear and the vestibular apparatus.

The cochlea is the portion of the auris interna related to hearing. The cochlea is a tapered tube-like structure which is coiled into a shape resembling a snail. The inside of the cochlea is divided into three regions, which is further defined by the position of the vestibular membrane and the basilar membrane. The portion above the vestibular membrane is the scala vestibuli, which extends from the oval window to the apex of the cochlea and contains perilymph fluid, an aqueous liquid low in potassium and high in sodium content. The basilar membrane defines the scala tympani region, which extends from the apex of the cochlea to the round window and also contains perilymph. The basilar membrane contains thousands of stiff fibers, which gradually increase in length from the round window to the apex of the cochlea. The fibers of the basement membrane vibrate when activated by sound. In between the scala vestibuli and the scala tympani is the cochlear duct, which ends as a closed sac at the apex of the cochlea. The cochlear duct contains endolymph fluid, which is similar to cerebrospinal fluid and is high in potassium.

The Organ of Corti, the sensory organ for hearing, is located on the basilar membrane and extends upward into the cochlear duct. The Organ of Corti contains hair cells, which have hairlike projections that extend from their free surface, and contacts a gelatinous surface called the tectorial membrane. Although hair cells have no axons, they are surrounded by sensory nerve fibers that form the cochlear branch of the vestibulocochlear nerve (cranial nerve VIII).

As discussed, the oval window, also known as the elliptical window communicates with the stapes to relay sound waves that vibrate from the tympanic membrane. Vibrations transferred to the oval window increases pressure inside the fluid-filled cochlea via the peri lymph and scala vestibuli/scala tympani, which in turn causes the membrane on the round window to expand in response. The concerted inward pressing of the oval window/outward expansion of the round window allows for the movement of fluid within the cochlea without a change of intra-cochlear pressure. However, as vibrations travel through the perilymph in the scala vestibuli, they create corresponding oscillations in the vestibular membrane. These corresponding oscillations travel through the endolymph of the cochlear duct, and transfer to the basilar membrane. When the basilar membrane oscillates, or moves up and down, the Organ of Corti moves along with it. The hair cell receptors in the Organ of Corti then move against the tectorial membrane, causing a mechanical deformation in the tectorial membrane. This mechanical deformation initiates the nerve impulse which travels via the vestibulocochlear nerve to the central nervous system, mechanically transmitting the sound wave received into signals that are subsequently processed by the central nervous system.

The auris interna is located in part within the osseous or bony labyrinth, an intricate series of passages in the temporal bone of the skull. The vestibular apparatus is the organ of balance and consists of the three semi-circular canals and the vestibule. The three semi-circular canals are arranged relative to each other such that movement of the head along the three orthogonal planes in space is detected by the movement of the fluid and subsequent signal processing by the sensory organs of the semi-circular canals, called the crista ampullaris. The crista ampullaris contains hair cells and supporting cells, and is covered by a dome-shaped gelatinous mass called the cupula. The hairs of the hair cells are embedded in the cupula. The semi-circular canals detect dynamic equilibrium, the equilibrium of rotational or angular movements.

When the head turns rapidly, the semicircular canals move with the head, but endolymph fluid located in the membranous semi-circular canals tends to remain stationary. The endolymph fluid pushes against the cupula, which tilts to one side. As the cupula tilts, it bends some of the hairs on the hair cells of the crista ampullaris, which triggers a sensory impulse. Because each semicircular canal is located in a different plane, the corresponding crista ampullaris of each semi-circular canal responds differently to the same movement of the head. This creates a mosaic of impulses that are transmitted to the central nervous system on the vestibular branch of the vestibulocochlear nerve. The central nervous system interprets this information and initiates the appropriate responses to maintain balance. Of importance in the central nervous system is the cerebellum, which mediates the sense of balance and equilibrium.

The vestibule is the central portion of the auris interna and contains mechanoreceptors bearing hair cells that ascertain static equilibrium, or the position of the head relative to gravity. Static equilibrium plays a role when the head is motionless or moving in a straight line. The membranous labyrinth in the vestibule is divided into two sac-like structures, the utricle and the saccule. Each structure in turn contains a small structure called a macula, which is responsible for maintenance of static equilibrium. The macula consists of sensory hair cells, which are embedded in a gelatinous mass (similar to the cupula) that covers the macula. Grains of calcium carbonate, called otoliths, are embedded on the surface of the gelatinous layer.

When the head is in an upright position, the hairs are straight along the macula. When the head tilts, the gelatinous mass and otoliths tilts correspondingly, bending some of the hairs on the hair cells of the macula. This bending action initiates a signal impulse to the central nervous system, which travels via the vestibular branch of the vestibulocochlear nerve, which in turn relays motor impulses to the appropriate muscles to maintain balance.

In some instances, the otic formulations described herein are placed in the outer ear. In some instances, the otic formulations described herein are placed in the middle or inner ear, including the cochlea and vestibular labyrinth: one option is to use a syringe/needle or pump and inject the formulation across the tympanic membrane (the eardrum). In some instances, for cochlear and vestibular labyrinth delivery, one option is to deliver the active ingredient across the round window membrane or even by microinjection directly into the auris interna also known as cochlear microperfusion.

### Diseases or Conditions of the Ear

In some embodiments, the otic formulations and compositions described herein are suitable for the treatment and/or prevention of diseases or conditions associated with the outer, middle, and/or inner ear. In some embodiments, the otic formulations and compositions described herein are suitable for the treatment and/or prevention of diseases or conditions associated with the inner ear. In some embodiments, the otic formulations and compositions described herein reduce, reverse and/or ameliorate symptoms of otic diseases or conditions, such as any one of these disclosed herein. These disorders or conditions have many causes, which include but are not limited to, infection, injury, inflammation, tumors, and adverse response to drugs or other chemical agents.

In some embodiments, the otic formulations and compositions described herein is useful for treating ear pruritus, otitis externa, otalgia, tinnitus, vertigo, ear fullness, hearing loss, or a combination thereof. In some embodiments, the otic formulations and compositions described herein are used to for the treatment and/or prevention of cochlear synaptopathy.

### Hair Cell Regeneration

Hair cells in the mammalian cochlea are important for hearing. The inner and outer hair cells in the Organ of Corti sense vibrations in cochlea fluid produced by sound and transduce these into auditory nerve responses that travel to the brain for sound to be perceived. Loss of hair cells has been implicated hearing loss caused by age, exposure to loud noise, ototoxic drugs, and genetic factors. In birds and amphibians, damage to hair cells triggers mechanisms that cause epithelial cells (supporting cells) in the cochlea to transdifferentiate into new hair cells and to divide and regenerate new supporting cells and hair cells to restore hearing. This ability to regenerate hair cells has been lost in mammals.

In some embodiments, the otic formulations or compositions described herein are useful for the regeneration of otic hair cells.

### Hearing Loss

Hearing loss is a partial or total impairment to hearing. Hearing loss is classified into three types, conductive hearing loss, sensorineural hearing loss, and mixed hearing loss. Conductive hearing loss occurs when sound is not conducted efficiently through the external auditory canal to the tympanic membrane or eardrum. In some embodiments, conductive hearing loss involves a reduction in sound level or the ability to hear faint sounds. Treatment involves corrective medical or surgical procedures. Sensorineural hearing loss occurs when there is damage to the cochlea (inner ear), or to the nerve pathways from the cochlea to the brain. This type of hearing loss generally leads to permanent hearing loss. Mixed hearing loss is a combination of conductive hearing loss and sensorineural hearing loss in which damage occurs along both the outer and inner ear regions.

The degree or severity of hearing loss is categorized into seven groups ranging from normal, slight, mild, moderate, moderately severe, severe to profound. In addition, hearing loss is stratified based on frequency in some instances. For example, a hearing loss that only affects the high tones is referred to as a high frequency hearing loss, whereas that which affects the low tones is referred to as a low frequency hearing loss. In some cases, hearing loss affects both high and low frequencies.

Hearing loss is often accompanied by additional causes and symptoms such as ceruminosis, otitis externa, otalgia, tinnitus and vertigo. In some embodiments, it has been shown that ceruminosis can decrease hearing acuity by 40-45 dB. Such impairment, especially in the geriatric population can cause difficulties in communication and even physical immobility. In some embodiments, the otic compositions and formulations disclosed herein are useful for the treatment of hearing loss.

### Cochlear Synaplopahly/Hearing-in-Noise/Speech-in-Noise

Approximately 38 million people in the United States have hearing loss as defined by hearing thresholds of 25 dB or greater using pure-tone audiometry in quiet (Goman and Lin 2016). In addition, difficulty with hearing-in-noise or the inability to understand speech-in-noise are common complaints of people with sensorineural hearing loss and those who wear hearing aids (Lopez-Poveda et al 2017). It is estimated that an additional 26 million adults in the United States with normal audiometric thresholds have difficulty hearing in noisy environments (Tremblay et al 2015). Difficulty hearing-in-noise is more common in older compared to younger individuals, with impairment increasing in prevalence beginning in middle age, and more men than women are typically affected (Moore et al 2014).

Accurate speech perception is vital for successful communication, good social participation, and a high quality of life (Ciesla et al 2016). Patients with unilateral sensorineural hearing loss often experience communication difficulties in spite of having normal hearing in one ear, with hearing in background noise one of the most challenging listening situations (American Academy of Audiology Clinical Practice Guidelines 2015). Individuals who demonstrated improved speech perception in noisy environments after hearing aid fitting also reported better quality of life across all assessed domains (physical, psychological, environmental, self assessment/quality of life, and social relationships), with the largest improvement in social relationships (Garcia et al 2016). Not surprisingly, individuals who are able to listen better are better socializers, participate more actively in groups, and avoid social isolation (Garcia et al 2016).

In addition, hearing impairment is consistently related to accelerated cognitive decline and increased dementia risk in older adults (Loughrey et al 2017). Potential pathways linking hearing loss to cognitive decline include sensory degradation, cognitive resource depletion, and social isolation (Fulton et al 2015). Cochlear implants have been reported to improve neurocognitive skills including executive function in older adults with hearing impairment (Volter et al 2018). A study of the effects of hearing treatment and rehabilitation on reducing cognitive decline in older adults with mild-to-moderate hearing impairment is underway (Deal et al 2018). It is possible that improving hearing in the hearing impaired could reduce cognitive decline in at-risk patients and provide significant clinical, social, and public health impact. Sensorineural hearing loss results from damage to the cochlea and is typically

associated with noise exposure (noise-induced hearing loss) or aging (age-related hearing loss) (Cunningham and Tucci 2017). Under normal hearing circumstances, sound-induced vibrations are transduced by sensory hair cells into electrical signals in cochlear neurons that relay encoded information to the brain (Fettiplace 2017). Hair cell damage is a key contributor to sensorineural hearing loss, as defined by the audiogram, which measures pure-tone detection in quiet in the frequency range of 0.25 to 8 kHz.

Emerging evidence in animals and humans suggests that synaptic connections between inner hair cells and spiral ganglion neurons in the cochlea can be lost after noise exposure and with aging, even without notable hair cell loss (Kujawa and Liberman 2009; Wu et al 2018). In animal studies, the loss of these synapses, referred to as cochlear synaptopathy, occurs in the absence of detectable changes in auditory thresholds and has been proposed as a mechanism that could underlie speech-in-noise difficulties in humans with otherwise normal audiometric thresholds. In addition, cochlear synaptopathy may be one of the earliest manifestations of future sensorineural hearing loss in which synaptic loss co-occurs with hair cell loss (Bramhall et al 2019). Restoring these synapses therapeutically, therefore, may have the potential to treat speech-in-noise hearing impairment and reduce the risk of sensorineural hearing loss.

### Sensorineural Hearing Loss

Sensorineural hearing loss is a type of hearing loss which results from defects (congenital and acquired) in the vestibulocochlear nerve (also known as cranial nerve VIII), or sensory cells of the inner ear. The majority of defects of the inner ear are defects of otic hair cells.

Aplasia of the cochlea, chromosomal defects, and congenital cholesteatoma are examples of congenital defects which result in sensorineural hearing loss. By way of non-limiting example, inflammatory diseases (e.g. suppurative labyrinthitis, meningitis, mumps, measles, viral syphilis, and autoimmune disorders), Meniere's Disease, exposure to ototoxic drugs (e.g. aminoglycosides, loop diuretics, antimetabolites, salicylates, and cisplatin), physical trauma, presbycusis, and acoustic trauma (prolonged exposure to sound in excess of 90 dB) all result in acquired sensorineural hearing loss.

If the defect resulting in sensorineural hearing loss is a defect in the auditory pathways, the sensorineural hearing loss is called central hearing loss. If the defect resulting in sensorineural hearing loss is a defect in the auditory pathways, the sensorineural hearing loss is called cortical deafness.

In some instances, sensorineural hearing loss occurs when the components of the auris interna or accompanying neural components are affected, and contain a neural, i.e. when the auditory nerve or auditory nerve pathways in the brain are affected, or sensory component. Sensory hearing loss are hereditary, or it are caused by acoustic trauma (i.e. very loud noises), a viral infection, drug-induced or Meniere's disease. In some instances, neural hearing loss occurs as a result of brain tumors, infections, or various brain and nerve disorders, such as stroke. Some hereditary diseases, such as Refsum disease (defective accumulation of branched fatty acids), also cause neural disorders affecting hearing loss. Auditory nerve pathways are damaged by demyelinating diseases, e.g. idiopathic inflammatory demyelinating disease (including multiple sclerosis), transverse myelitis, Devic's disease, progressive multifocal leukoencephalopathy, Guillain-Barre syndrome, chronic inflammatory demyelinating polyneuropathy and anti-MAG peripheral neuropathy.

The incidence of sudden deafness, or sensorineural hearing loss, occurs in about 1 in 5000 individuals, and are caused by viral or bacterial infections, e.g. mumps, measles, influenza, chickenpox, cytomegalovirus, syphilis or infectious mononucleosis, or physical injury to the inner ear organ. In some cases, no cause is identified. In some cases, tinnitus and vertigo accompany sudden deafness, which subsides gradually. Oral corticosteroids are frequently prescribed to treat sensorineural hearing loss. In some cases, surgical intervention is necessary. Other treatments include AM-101 and AM-111, compounds under development for the treatment of auris interna tinnitus and acute sensorineural hearing loss. (Auris Medical AG, Basel, Switzerland).

### Noise Induced Hearing Loss

Noise induced hearing loss (NIHL) is caused upon exposure to sounds that are too loud or loud sounds that last a long time. In some instances, hearing loss occurs from prolonged exposure to loud noises, such as loud music, heavy equipment or machinery, airplanes, or gunfire. Long or repeated or impulse exposure to sounds at or above 85 decibels cause hearing loss in some cases. NIHL causes damage to the hair cells and/or the auditory nerve. The hair cells are small sensory cells that convert sound energy into electrical signals that travel to the brain. In some cases, impulse sound results in immediate hearing loss that is permanent. This kind of hearing loss are accompanied by tinnitus—a ringing, buzzing, or roaring in the ears or head—which subsides over time in some cases. Hearing loss and tinnitus are experienced in one or both ears, and tinnitus continue constantly or occasionally throughout a lifetime in some instances. Permanent damage to hearing loss is often diagnosed. Continuous exposure to loud noise also damages the structure of hair cells, resulting in hearing loss and tinnitus, although the process occurs more gradually than for impulse noise.

In some embodiments, an otoprotectant reverses, reduces, or ameliorates NIHL. Examples of otoprotectants that treat or prevent NIHL include, but are not limited to, D-methionine, L-methionine, ethionine, hydroxyl methionine, methioninol, amifostine, mesna (sodium 2-sulfanylethanesulfonate), a mixture of D and L methionine, normethionine, homomethionine, S-adenosyl-L-methionine), diethyldithiocarbamate, ebselen (2-phenyl-1,2-benzisoselenazol-3(2H)-one), sodium thiosulfate, AM-111 (a cell permeable JNK inhibitor, (Laboratoires Auris SAS)), leucovorin, leucovorin calcium, dexrazoxane, or combinations thereof.

### Presbycusis (Age Related Hearing Loss)

Presbycusis (age related hearing loss (ARHL)) is the progressive bilateral loss of hearing that results from aging. Most hearing loss occurs at higher frequencies (i.e. frequencies above 15 or 16 Hz) making it difficult to hear a female voice (as opposed to male voice), and an inability to differentiate between high-pitched sounds (such as “s” and “th”). It is difficult to filter out background noise. The disorder is most often treated by the implantation of a hearing aid and/or the administration of pharmaceutical agents which prevent the buildup of ROS.

The disorder is caused by changes in the physiology of the inner ear, the middle ear, and/or the VIII nerve. Changes in the inner ear resulting in presbycusis include epithelial atrophy with loss of otic hair cells and/or stereocilia, atrophy of nerve cells, atrophy of the stria vascularis, and the thickening/stiffening of the basilar membrane. Additional changes which contribute to presbycusis include the accumulation of defects in the tympanic membrane and the ossicles.

In some instances, changes leading to presbycusis occur due to the accumulation of mutations in DNA, and mutations in mitochondrial DNA; however, the changes are exacerbated by exposure to loud noise, exposure to ototoxic agents, infections, and/or the lessening of blood flow to the ear. The latter is attributable to atherosclerosis, diabetes, hypertension, and smoking.

Presbycusis, or age-related hearing loss, occurs as a part of normal aging, and occurs as a result of degeneration of the receptor cells in the spiral Organ of Corti in the auris interna. Other causes are also attributed to a decrease in a number of nerve fibers in the vestibulocochlear nerve, as well as a loss of flexibility of the basilar membrane in the cochlea. Most commonly, it arises from changes in the inner ear as one ages, but it also results from changes in the middle ear, or from complex changes along the nerve pathways from the ear to the brain. Certain medical conditions and medications also play a role. In some instances, presbycusis results from a gradual loss of spiral ganglion neuron afferent fibers and their synapses with hair cells (ribbon synapses), causing a disconnection between the sensory cells that detect sound and the auditory nerve that transmits this information to the auditory brain. Loss of spiral ganglion neurons and hair cells also occurs. In some cases, prior exposure to loud noise or other otic insults exacerbates this ageing process, leading to an accelerated loss of hearing. Presbycusis also involves “hidden hearing loss”, an inability to detect sound against a background noise (“speech-in-noise”) despite a lack of marked changes in hearing thresholds. These more subtle decrements in hearing have been associated with a loss of spiral ganglion neuron afferent fibers and their synaptic connections with hair cells (ribbon synapses).

In some embodiments, the agents described herein repair ribbon synapses and restores hearing function. In some embodiments, the ribbon synapses are damaged due to noise trauma or exposure.

### Ototoxicity

Ototoxicity refers to hearing loss caused by a toxin. The hearing loss are due to trauma to otic hair cells, the cochlea, and/or the cranial nerve VIII. Multiple drugs are known to be ototoxic. Often ototoxicity is dose-dependent. It is permanent or reversible upon withdrawal of the drug.

Known ototoxic drugs include, but are not limited to, the aminoglycoside class of antibiotics (e.g., gentamicin, and amikacin), some members of the macrolide class of antibiotics (e.g., erythromycin), some members of the glycopeptide class of antibiotics (e.g., vancomycin), salicylic acid, nicotine, some chemotherapeutic agents (e.g., actinomycin, bleomycin, cisplatin, carboplatin and vincristine), and some members of the loop diuretic family of drugs (e.g., furosemide).

Cisplatin and the aminoglycoside class of antibiotics induce the production of reactive oxygen species (“ROS”). ROS damages cells directly by damaging DNA, polypeptides, and/or lipids. Antioxidants prevent damage of ROS by preventing their formation or scavenging free radicals before they damage the cell. Both cisplatin and the aminoglycoside class of antibiotics are also thought to damage the ear by binding melanin in the stria vascularis of the inner ear.

Salicylic acid is classified as ototoxic as it inhibits the function of the polypeptide prestin. Prestin mediates outer otic hair cell motility by controlling the exchange of chloride and carbonate across the plasma membrane of outer otic hair cells. It is only found in the outer otic hair cells, not the inner otic hair cells. Accordingly, in some embodiments, the use of the controlled release auris-compositions described herein, ameliorates or lessens ototoxic effects of chemotherapy, including but not limited to cisplatin treatment, aminoglycoside or salicylic acid administration, or other ototoxic agents.

### Excitotoxicity

Excitotoxicity refers to the death or damaging of neurons and/or otic hair cells by glutamate and/or similar substances.

Glutamate is the most abundant excitatory neurotransmitter in the central nervous system. Pre-synaptic neurons release glutamate upon stimulation. It flows across the synapse, binds to receptors located on post-synaptic neurons, and activates these neurons. The glutamate receptors include the NMDA, AMPA, and kainate receptors. Glutamate transporters are tasked with removing extracellular glutamate from the synapse. Certain events (e.g. ischemia or stroke) damage the transporters. This results in excess glutamate accumulating in the synapse. Excess glutamate in synapses results in the over-activation of the glutamate receptors.

The AMPA receptor is activated by the binding of both glutamate and AMPA. Activation of certain isoforms of the AMPA receptor results in the opening of ion channels located in the plasma membrane of the neuron. When the channels open, Na⁺ and Ca²⁺ ions flow into the neuron and ions flow out of the neuron.

The NMDA receptor is activated by the binding of both glutamate or NMDA together with a co-agonist glycine or D-serine. Activation of the NMDA receptor, results in the opening of ion channels located in the plasma membrane of the neuron. However, these channels are blocked by Mg²⁺ ions. Activation of the AMPA receptor results in the expulsion of Mg²⁺ ions from the ion channels into the synapse. When the ion channels open, and the Mg²⁺ ions evacuate the ion channels, Na⁺ and Ca²⁺ ions flow into the neuron, and K⁺ ions flow out of the neuron.

Excitotoxicity occurs when the NMDA receptor and AMPA receptors are over-activated by the binding of excessive amounts of ligands, for example, abnormal amounts of glutamate. The over-activation of these receptors causes excessive opening of the ion channels under their control. This allows abnormally high levels of Ca²⁺ and Na⁺ to enter the neuron. The influx of these levels of Ca²⁺ and Na⁺ into the neuron causes the neuron to fire more often, resulting in a rapid buildup of free radicals and inflammatory compounds within the cell. The free radicals eventually damage the mitochondria, depleting the cell's energy stores. Furthermore, excess levels of Ca²⁺ and Na⁺ ions activate excess levels of enzymes including, but not limited to, phospholipases, endonucleases, and proteases. The over-activation of these enzymes results in damage to the cytoskeleton, plasma membrane, mitochondria, and DNA of the sensory neuron. Local otic administration

Also provided herein are methods, formulations, and compositions for local delivery of therapeutic agents (otic agents) to auris externa, auris media, and/or auris interna structures. In some embodiments, local delivery of the therapeutic agent (otic agent) overcomes the toxic and attendant side effects of systemic delivery. In some embodiments, access to the vestibular and cochlear apparatus is through the auris media and includes the round window membrane, the oval window/stapes footplate, the annular ligament and through the otic capsule/temporal bone.

Provided herein, in certain embodiments, are otic formulations and compositions that remain in contact with the target auditory surfaces (e.g., the round window) for extended periods of time. In some embodiments, the otic formulations and compositions further comprise mucoadhesives that allow the otic formulations to adhere to otic mucosal surfaces. In some instances, the formulations and compositions described herein avoid attenuation of therapeutic benefit due to drainage or leakage of active agents via the eustachian tube.

In some embodiments, the localized treatment of the auris externa, auris media and/or auris interna affords the use of previously undesired therapeutic agents, including agents with poor PK profiles, poor uptake, low systemic release and/or toxicity issues. In some embodiments, localized targeting of the otic agent formulations and compositions reduces the risk of adverse effects with previously characterized toxic or ineffective therapeutic agents (otic active agents). Accordingly, also contemplated within the scope of the embodiments described herein is the use of active agents and/or agents that have been previously rejected by practitioners because of adverse effects or ineffectiveness of the therapeutic agent (otic agent).

In some embodiments, specifically targeting the auris externa, auris media and/or auris interna structures avoids the adverse side effects usually associated with systemic treatment. In some embodiments, the otic formulations and compositions described herein are controlled release therapeutic agent formulations and compositions that treat otic disorders by providing a constant, variable and/or extended source of a therapeutic agent (otic agent) to the individual or patient suffering from an otic disorder, thereby reducing or eliminating the variability of treatment. Accordingly, one embodiment disclosed herein is to provide a formulation or composition that enables at least one therapeutic agent (otic agent) to be released in therapeutically effective doses either at variable or constant rates such as to ensure a continuous release of the at least one therapeutic agent (otic agent). In some embodiments, the therapeutic agents (otic agents) disclosed herein are administered as an immediate release formulation or composition. In other embodiments, the therapeutic agents (otic agents) are administered as a controlled release formulation, released either continuously or in a pulsatile manner, or variants of both. In still other embodiments, the therapeutic agent (otic agent) formulation or composition is administered as both an immediate release and controlled release formulation or composition, released either continuously or in a pulsatile manner, or variants of both. The release is optionally dependent on environmental or physiological conditions, for example, the external ionic environment (see, e.g. Oros® release system, Johnson & Johnson).

In addition, the otic compositions or formulations included herein also optionally include carriers, adjuvants, such as preserving, stabilizing, wetting or emulsifying agents, solution promoters, salts for regulating the osmotic pressure, and/or buffers. Such carriers, adjuvants, and other excipients are compatible with the environment in the auris externa, auris media and/or auris interna. Accordingly, specifically contemplated are carriers, adjuvants and excipients that lack ototoxicity or are minimally ototoxic in order to allow effective treatment of the otic disorders contemplated herein with minimal side effects in the targeted regions or areas. To prevent ototoxicity, otic compositions or formulations disclosed herein are optionally targeted to distinct regions of the auris externa, auris media and/or auris interna, including but not limited to the tympanic cavity, vestibular bony and membranous labyrinths, cochlear bony and membranous labyrinths and other anatomical or physiological structures located within the auris interna.

### Treatment

Provided herein are otic formulations and compositions suitable for the treatment of any otic condition, disease or disorder (e.g., outer, middle and/or inner ear disorders) described herein, comprising administration of a therapeutic agent (otic agent) described herein to an individual or patient in need thereof. The formulations and compositions described herein are suitable for the treatment of any disease described herein. In some instances, the treatment is long-term treatment for chronic recurring disease. In some instances, the treatment is prophylactic administration of an otic formulation described herein for the treatment of any otic disease or disorder described herein. In some instances, prophylactic administration avoids occurrence of disease in individuals suspected of having a disease or in individuals genetically predisposed to an otic disease or disorder. In some instances the treatment is preventive maintenance therapy. In some instances, preventive maintenance therapy avoids recurrence of a disease.

In some instances, because of their otic compatibility and improved sterility, the otic formulations and compositions described herein are safe for long-term administration. In some embodiments, the otic formulations and compositions described herein have very low ototoxicity.

In some embodiments, the otic formulations and compositions described herein provide a sustained release of a therapeutic agent (otic agent) for a period of at least one day, three days, five days, one week, two weeks, three weeks, a month, two months, three months, four months, five months, six months, or a year. In some embodiments, the otic formulations and compositions described herein provide a steady sustained release of a therapeutic agent (otic agent) for a period of at least three days. In some embodiments, the otic formulations and compositions described herein provide a steady sustained release of a therapeutic agent (otic agent) for a period of at least five days. In some embodiments, the otic formulations and compositions described herein provide a steady sustained release of a therapeutic agent (otic agent) for a period of at least one week. In some embodiments, the otic formulations and compositions described herein provide a steady sustained release of a therapeutic agent (otic agent) for a period of at least two weeks. In some embodiments, the otic formulations and compositions described herein provide a steady sustained release of a therapeutic agent (otic agent) for a period of at least three weeks. In some embodiments, the otic formulations and compositions described herein provide a steady sustained release of a therapeutic agent (otic agent) for a period of at least a month. In some embodiments, the otic formulations and compositions described herein provide a steady sustained release of a therapeutic agent (otic agent) for a period of at least two months. In some embodiments, the otic formulations and compositions described herein provide a steady sustained release of a therapeutic agent (otic agent) for a period of at least three months. In some embodiments, the otic formulations and compositions described herein provide a steady sustained release of a therapeutic agent (otic agent) for a period of at least four months. In some embodiments, the otic formulations and compositions described herein provide a steady sustained release of a therapeutic agent (otic agent) for a period of at least five months. In some embodiments, the otic formulations and compositions described herein provide a steady sustained release of a therapeutic agent (otic agent) for a period of at least six months. In some embodiments, the otic formulations and compositions described herein provide a steady sustained release of a therapeutic agent (otic agent) for a period of at least a year.

In some embodiments, the otic formulations and compositions described herein provide a sustained release of a therapeutic agent (otic agent) for a period of about a day, three days, five days, one week, two weeks, three weeks, a month, two months, three months, four months, five months, six months, or a year. In some embodiments, the otic formulations and compositions described herein provide a steady sustained release of a therapeutic agent (otic agent) for a period of about three days. In some embodiments, the otic formulations and compositions described herein provide a steady sustained release of a therapeutic agent (otic agent) for a period of about five days. In some embodiments, the otic formulations and compositions described herein provide a steady sustained release of a therapeutic agent (otic agent) for a period of about one week. In some embodiments, the otic formulations and compositions described herein provide a steady sustained release of a therapeutic agent for a period of about two weeks. In some embodiments, the otic formulations and compositions described herein provide a steady sustained release of a therapeutic agent (otic agent) for a period of about three weeks. In some embodiments, the otic formulations and compositions described herein provide a steady sustained release of a therapeutic agent (otic agent) for a period of about a month. In some embodiments, the otic formulations and compositions described herein provide a steady sustained release of a therapeutic agent (otic agent) for a period of about two months. In some embodiments, the otic formulations and compositions described herein provide a steady sustained release of a therapeutic agent (otic agent) for a period of about three months. In some embodiments, the otic formulations and compositions described herein provide a steady sustained release of a therapeutic agent (otic agent) for a period of about four months. In some embodiments, the otic formulations and compositions described herein provide a steady sustained release of a therapeutic agent (otic agent) for a period of about five months. In some embodiments, the otic formulations and compositions described herein provide a steady sustained release of a therapeutic agent (otic agent) for a period of about six months. In some embodiments, the otic formulations and compositions described herein provide a steady sustained release of a therapeutic agent (otic agent) for a period of about a year.

In one aspect, provided herein are controlled release compositions and formulations to treat and/or prevent diseases or conditions associated with the ear. In some instances, these diseases or conditions associated with the ear include the outer, the middle ear and/or inner ear.

In some embodiments, the disease or condition is cochlear synaptopathy, hearing loss, or a combination thereof. In some embodiment, the disease or condition is hearing-in-noise difficulties. In some embodiment, the disease or condition is speech-in-noise impairement. In some embodiments, the diseases or conditions include sensorineural hearing loss, noise induced hearing loss, presbycusis (age related hearing loss), ototoxicity, excitotoxicity, or combinations thereof.

The etiology of several ear diseases or disorders consists of a syndrome of progressive hearing loss, including noise induced hearing loss and age-related hearing loss, cochlear synaptopathy, hearing-in-noise difficulties, and speech-in-noise impairment. These disorders have many causes, such as infection, exposure to noise, injury, inflammation, tumors, and/or adverse response to drugs or other chemical agents.

In some embodiments, the otic formulations and compositions described herein have pH and osmolarity that are auris-acceptable. In some embodiments, the otic formulations and compositions described herein meet the stringent sterility requirements described herein and are compatible with the endolymph and/or the perilymph. Pharmaceutical agents that are used in conjunction with the formulations and compositions disclosed herein include agents that ameliorate or lessen otic disorders, including auris interna disorders, and their attendant symptoms, which include but are not limited to hearing loss, nystagmus, vertigo, tinnitus, inflammation, swelling, infection and congestion. Otic disorders have many causes and include infection, injury, inflammation, tumors and adverse response to drugs or other chemical agents that are responsive to the pharmaceutical agents disclosed herein. In some embodiments, pharmaceutically active metabolites, salts, polymorphs, prodrugs, analogues, and derivatives of the otic agents disclosed herein are used in the formulations.

For some embodiments, wherein the formulation or composition is designed such that the active ingredient has limited or no systemic release, therapeutic agents that produce systemic toxicities (e.g., liver toxicity) or have poor PK characteristics (e.g. short half-life) are also optionally used. Thus, in some embodiments, therapeutic agents that have been previously shown to be toxic, harmful or non-effective during systemic application, for example through toxic metabolites formed after hepatic processing, toxicity of the drug in particular organs, tissues or systems, through high levels needed to achieve efficacy, through the inability to be released through systemic pathways or through poor PK characteristics, are useful. The formulations and compositions disclosed herein are contemplated to be targeted directly to otic structures where treatment is needed; for example, one embodiment contemplated is the direct application of the otic formulations disclosed herein onto the round window membrane or the crista fenestrae cochlea of the auris interna, allowing direct access and treatment of the auris interna, or inner ear components. In other embodiments, the otic formulations and compositions disclosed herein are applied directly to the oval window. In yet other embodiments, direct access is obtained through microinjection directly into the auris interna, for example, through cochlear microperfusion. Such embodiments also optionally comprise a drug delivery device, wherein the drug delivery device delivers the otic formulations through use of a needle and syringe, a pump, a microinjection device, a spongy material or any combination thereof.

In still other embodiments, application of any otic formulation or composition described herein is targeted to the auris media through piercing of the intratympanic membrane and applying the otic agent formulation directly to the auris media structures affected, including the walls of the tympanic cavity or auditory ossicles. In some embodiments, the auris active agent formulations and compositions disclosed herein are confined to the targeted auris media structure, and will not be lost, for example, through diffusion or leakage through the eustachian tube or pierced tympanic membrane.

In some embodiments, the otic formulations and compositions disclosed herein are delivered to the auris externa in any suitable manner, including by cotton swab, injection or ear drops. Also, in other embodiments, the otic formulations and compositions described herein are targeted to specific regions of the auris externa by application with a needle and syringe, a pump, a microinjection device, a spongy material, or any combination thereof. For example, in the case of treatment of otitis externa, antimicrobial agent formulations disclosed herein are delivered directly to the ear canal, where they are retained, thereby reducing loss of the active agents from the target ear structure by drainage or leakage.

### Growth Factors

Contemplated for use with the formulations disclosed herein are agents that modulate the degeneration of neurons and/or hair cells of the auris, promote the survival and/or growth of neurons and/or hair cells of the auris, and agents for treating or ameliorating hearing loss or reduction resulting from destroyed, stunted, malfunctioning, damaged, fragile or missing hairs in the inner ear. Accordingly, some embodiments incorporate the use of agents which promote the survival of neurons and otic hair cells, and/or the growth of neurons and otic hair cells. In some embodiments, the agent which promotes the survival of otic hair cells is a growth factor. In some embodiments, the growth factor is a neurotroph. In certain instances, neurotrophs are growth factors which prevent cells from initiating apoptosis, repair damaged neurons and otic hair cells, and/or induce differentiation in progenitor cells. In some embodiments, the growth factor is brain-derived neurotrophic factor (BDNF).

In some embodiments, the growth factor is a neurotroph. In certain instances, neurotrophs are growth factors which prevent cells from initiating apoptosis, repair damaged neurons and otic hair cells, and/or induce differentiation in progenitor cells. In some embodiments, the neurotroph is brain-derived neurotrophic factor (BDNF).

In some embodiments, the neurotroph is BDNF. In certain instances, BDNF is a neurotroph which promotes the survival of existing neurons (e.g. spiral ganglion neurons), and otic hair cells by repairing damaged cells, inhibiting the production of ROS, and inhibiting the induction of apoptosis. In certain embodiments, it also promotes the differentiation of neural and otic hair cell progenitors.

In some embodiments, the neurotroph is a Trk agonist, such as a TrkB or TrkC agonist. For example, BDNF activate TrkB and TrkC on spiral ganglion neurons to prompt survival, neurite growth, and synapse formation.

### Devices

Also contemplated herein are the use of devices for the delivery of the pharmaceutical formulations and compositions disclosed herein, or alternatively for the measurement or surveillance of the function of the auris formulations disclosed herein. For example, in one embodiment pumps, osmotic devices or other means of mechanically delivering pharmaceutical formulations and compositions are used for the delivery of the pharmaceutical formulations disclosed herein. Reservoir devices are optionally used with the pharmaceutical drug delivery units, and reside either internally along with the drug delivery unit, or externally of the auris structures.

Other embodiments contemplate the use of mechanical or imaging devices to monitor or survey the hearing, balance or other auris disorder. For example, magnetic resonance imaging (MRI) devices are specifically contemplated within the scope of the embodiments, wherein the MRI devices (for example, 3 Tesla MRI devices) are capable of evaluating Ménière Disease progression and subsequent treatment with the pharmaceutical formulations disclosed herein. See, Carfrae et al. Laryngoscope 118:501-505 (March 2008). Whole body scanners, or alternatively cranial scanners, are contemplated, as well as higher resolution (7 Tesla, 8 Tesla, 9.5 Tesla or 11 Tesla for humans) are optionally used in MRI scanning.

### Visualization of Otic Formulations

Also provided herein in some embodiments are otic formulations and compositions that comprise a dye (e.g., a Trypan blue dye, Evans blue dye) or other tracer compound. In some instances, addition of an auris-compatible dye to an otic formulation or composition described herein aids visualization of any administered formulation or composition in an ear (e.g., a rodent ear and/or a human ear). In certain embodiments, an otic formulation or composition comprising a dye or other tracer compound eliminates the need for invasive procedures that are currently used in animal models to monitor the concentrations of drugs in the endolymph and/or perilymph.

In some instances, intratympanic injections require the need of a specialist and the formulation or composition needs to be delivered to a specific site of the ear to maximize efficiency of the medication delivered. In certain instances, a visualization technique for any formulation or composition described herein allows for visualization of a dosing site (e.g., the round window) so that the medication is applied in the proper place. In some instances, a formulation or composition comprising a dye allows visualization of the formulation or composition during administration of the formulation to an ear (e.g., a human ear), ensures that the medication will be delivered at the intended site, and avoids any complications due to incorrect placement of a formulation or composition. The inclusion of a dye to help enhance the visualization of the formulation or composition when applied, and the ability to visually inspect the location of the formulation or composition after administration without further intervention, represents an advance over currently available methods for testing intratympanic therapeutics in animal models and/or human trials. In some embodiments, dyes that are compatible with the otic compositions described herein include Evans blue (e.g., 0.5% of the total weight of an otic formulation), Methylene blue (e.g., 1% of the total weight of an otic formulation), Isosulfan blue (e.g., 1% of the total weight of an otic formulation), Trypan blue (e.g., 0.15% of the total weight of an otic formulation), and/or indocyanine green (e.g., 25 mg/vial). Other common dyes, e.g., FD&C red 40, FD&C red 3, FD&C yellow 5, FD&C yellow 6, FD&C blue 1, FD&C blue2, FD&C green 3, fluorescence dyes (e.g., Fluorescein isothiocyanate, rhodamine, Alexa Fluors, DyLight Fluors) and/or dyes that are visualizable in conjunction with non-invasive imaging techniques such as MRI, CAT scans, PET scans.or the like (e.g., Gadolinium-based MRI dyes, iodine-base dyes, barium-based dyes or the like) are also contemplated for use with any otic formulation or composition described herein. Other dyes that are compatible with any formulation or composition described herein are listed in the Sigma-Aldrich catalog under dyes (which is included herein by reference for such disclosure). In some embodiments, concentration of a dye in any otic formulation described herein is less than 2%, less than 1.5%, less than 1%, less than 0.5%, less than 0.25%, less than 0.1%, or less than 100 ppm of the total weight and/or volume of any formulation or composition described herein.

In certain embodiments of such auris-compatible formulations or compositions that comprise a dye, the ability to visualize a controlled release otic formulation or composition comprising a dye in an ear meets a long standing need for suitable testing methods that are applicable to the development of intratympanic otic formulations or compositions suitable for human use. In certain embodiments of such auris-compatible formulations or compositions that comprise a dye, the ability to visualize a controlled release otic formulation or composition comprising a dye allows for testing of any otic formulation described herein in human clinical trials.

### Auris-Acceptable Gel Formulations/Compositions

In some embodiments, the auris-acceptable formulations or compositions described herein are gel formulations or gel compositions.

In some embodiments, the otic gel formulations or compositions that include at least therapeutic agent and a pharmaceutically acceptable diluent(s), excipient(s), or carrier(s). In some embodiments, the otic gel formulations or compositions include other medicinal or pharmaceutical agents; carriers; adjuvants; preserving, stabilizing, wetting or emulsifying agents; solution promoters; salts for regulating the osmotic pressure; and/or buffers. In some embodiments, the otic gel formulations or compositions comprises (i) a therapeutic agent, (ii) a gelling and viscosity enhancing agent, (iii) a pH adjusting agent, and (iv) sterile water.

Gels, sometimes referred to as jellies, have been defined in various ways. For example, the United States Pharmacopoeia defines gels as semisolid systems consisting of either suspensions made up of small inorganic particles or large organic molecules interpenetrated by a liquid. Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums (e.g., tragacanth). In some embodiments, single-phase gels are generally aqueous but will also be made using alcohols and oils. Two-phase gels consist of a network of small discrete particles.

Gels can also be classified as being hydrophobic or hydrophilic. In certain embodiments, the base of a hydrophobic gel consists of a liquid paraffin with polyethylene or fatty oils gelled with colloidal silica or aluminum or zinc soaps. In contrast, the base of hydrophilic gels usually consists of water, glycerol, or propylene glycol gelled with a suitable gelling agent (e.g., tragacanth, starch, cellulose derivatives, carboxyvinylpolymers, and magnesium-aluminum silicates). In certain embodiments, the rheology of the formulations or devices disclosed herein is pseudo plastic, plastic, thixotropic, or dilatant.

In one embodiment the enhanced viscosity auris-acceptable formulation described herein is not a liquid at room temperature. In certain embodiments, the enhanced viscosity formulation is characterized by a phase transition between room temperature and body temperature (including an individual with a serious fever, e.g., up to about 42° C.). In some embodiments, the phase transition occurs at about 1° C. below body temperature, at about 2° C. below body temperature, at about 3° C. below body temperature, at about 4° C. below body temperature, at about 6° C. below body temperature, at about 8° C. below body temperature, or at about 10° C. below body temperature. In some embodiments, the phase transition occurs at about 15° C. below body temperature, at about 20° C. below body temperature, or at about 25° C. below body temperature. In specific embodiments, the gelation temperature (Tgel) of a formulation described herein is about 20° C., about 25° C., or about 30° C. In certain embodiments, the gelation temperature (Tgel) of a formulation described herein is about 35° C. or about 40° C. In one embodiment, administration of any formulation described herein at about body temperature reduces or inhibits vertigo associated with intratympanic administration of otic formulations. Included within the definition of body temperature is the body temperature of a healthy individual or an unhealthy individual, including an individual with a fever (up to ˜42° C.). In some embodiments, the pharmaceutical formulations or devices described herein are liquids at about room temperature and are administered at or about room temperature, reducing or ameliorating side effects such as, for example, vertigo.

Polymers composed of polyoxypropylene and polyoxyethylene form thermoreversible gels when incorporated into aqueous solutions. These polymers have the ability to change from the liquid state to the gel state at temperatures close to body temperature, therefore allowing useful formulations that are applied to the targeted auris structure(s). The liquid state-to-gel state phase transition is dependent on the polymer concentration and the ingredients in the solution.

Poloxamer 407 (PF-127) is a nonionic surfactant composed of polyoxyethylene-polyoxypropylene copolymers. Other poloxamers include 188 (F-68 grade), 237 (F-87 grade), and 338 (F-108 grade). Aqueous solutions of poloxamers are stable in the presence of acids, alkalis, and metal ions. PF-127 is a commercially available polyoxyethylene-polyoxypropylene triblock copolymer also known as Poloxamer 407 or P407. The polymer can be further purified by suitable methods that will enhance gelation properties of the polymer. It contains approximately 70% ethylene oxide, which accounts for its hydrophilicity. It is one of the series of poloxamer ABA block copolymers, whose members share the chemical formula shown below.

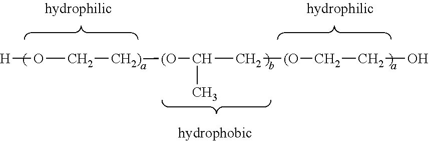

In some embodiments, the amount of thermoreversible polymer in any formulation described herein is about 0.01%, about 0.05%, about 0.1%, about 0.5%, about 1%, about 5%, about 10%, about 15%, about 20%, about 25%, about 30%, about 35%, or about 40% of the total weight of the formulation. In some embodiments, the amount of thermoreversible polymer in any formulation described herein is about 0.01%, about 0.05%, about 0.1%, about 0.5%, about 1%, about 5%, about 10%, about 11%, about 12%, about 13%, about 14%, about 15%, about 16%, about 17%, about 18%, about 19%, about 20%, about 21%, about 22%, about 23%, about 24%, or about 25% of the total weight of the formulation. In some embodiments, the amount of thermoreversible polymer (e.g., pluronic F127) in any formulation described herein is about 7.5% of the total weight of the formulation. In some embodiments, the amount of thermoreversible polymer (e.g., pluronic F127) in any formulation described herein is about 10% of the total weight of the formulation. In some embodiments, the amount of thermoreversible polymer (e.g., pluronic F127) in any formulation described herein is about 11% of the total weight of the formulation. In some embodiments, the amount of thermoreversible polymer (e.g., pluronic F127) in any formulation described herein is about 12% of the total weight of the formulation. In some embodiments, the amount of thermoreversible polymer (e.g., pluronic F127) in any formulation described herein is about 13% of the total weight of the formulation. In some embodiments, the amount of thermoreversible polymer (e.g., pluronic F127) in any formulation described herein is about 14% of the total weight of the formulation. In some embodiments, the amount of thermoreversible polymer (e.g., pluronic F127) in any formulation described herein is about 15% of the total weight of the formulation. In some embodiments, the amount of thermoreversible polymer (e.g., pluronic F127) in any formulation described herein is about 16% of the total weight of the formulation. In some embodiments, the amount of thermoreversible polymer (e.g., pluronic F127) in any formulation described herein is about 17% of the total weight of the formulation. In some embodiments, the amount of thermoreversible polymer (e.g., pluronic F127) in any formulation described herein is about 18% of the total weight of the formulation. In some embodiments, the amount of thermoreversible polymer (e.g., pluronic F127) in any formulation described herein is about 19% of the total weight of the formulation. In some embodiments, the amount of thermoreversible polymer (e.g., pluronic F127) in any formulation described herein is about 20% of the total weight of the formulation. In some embodiments, the amount of thermoreversible polymer (e.g., pluronic F127) in any formulation described herein is about 21% of the total weight of the formulation. In some embodiments, the amount of thermoreversible polymer (e.g., pluronic F127) in any formulation described herein is about 23% of the total weight of the formulation. In some embodiments, the amount of thermoreversible polymer (e.g., pluronic F127) in any formulation described herein is about 25% of the total weight of the formulation.

In an alternative embodiment, the thermogel is a PEG-PLGA-PEG triblock copolymer (Jeong et al, Nature (1997), 388:860-2; Jeong et al, J. Control. Release (2000), 63:155-63; Jeong et al, Adv. Drug Delivery Rev. (2002), 54:37-51). The polymer exhibits sol-gel behavior over a concentration of about 5% w/w to about 40% w/w. Depending on the properties desired, the lactide/glycolide molar ratio in the PLGA copolymer ranges from about 1:1 to about 20:1. The resulting coploymers are soluble in water and form a free-flowing liquid at room temperature but form a hydrogel at body temperature. A commercially available PEG-PLGA-PEG triblock copolymer is RESOMER RGP t50106 manufactured by Boehringer Ingelheim. This material is composed of a PGLA copolymer of 50:50 poly(DL-lactide-co-glycolide), is 10% w/w of PEG, and has a molecular weight of about 6000.

ReGel® is a tradename of MacroMed Incorporated for a class of low molecular weight, biodegradable block copolymers having reverse thermal gelation properties as described in U.S. Pat. Nos. 6,004,573, 6,117949, 6,201,072, and 6,287,588. It also includes biodegradable polymeric drug carriers disclosed in pending U.S. patent application Ser. Nos. 09/906,041, 09/559,799 and 10/919,603. The biodegradable drug carrier comprises ABA-type or BAB-type triblock copolymers, or mixtures thereof, wherein the A-blocks are relatively hydrophobic and comprise biodegradable polyesters or poly(orthoester)s, and the B-blocks are relatively hydrophilic and comprise polyethylene glycol (PEG), said copolymers having a hydrophobic content of between 50.1 to 83% by weight and a hydrophilic content of between 17 to 49.9% by weight, and an overall block copolymer molecular weight of between 2000 and 8000 Daltons. The drug carriers exhibit water solubility at temperatures below normal mammalian body temperatures and undergo reversible thermal gelation to then exist as a gel at temperatures equal to physiological mammalian body temperatures. The biodegradable, hydrophobic A polymer block comprises a polyester or poly(ortho ester), in which the polyester is synthesized from monomers selected from the group consisting of D,L-lactide, D-lactide, L-lactide, D,L-lactic acid, D-lactic acid, L-lactic acid, glycolide, glycolic acid, ϵ-caprolactone, ϵ-hydroxyhexanoic acid, γ-butyrolactone, γ-hydroxybutyric acid, δ-valerolactone, δ-hydroxyvaleric acid, hydroxybutyric acids, malic acid, and copolymers thereof and having an average molecular weight of between about 600 and 3000 Daltons. The hydrophilic B-block segment is preferably polyethylene glycol (PEG) having an average molecular weight of between about 500 and 2200 Daltons.

Additional biodegradable thermoplastic polyesters include AtriGel ® (provided by Atrix Laboratories, Inc.) and/or those disclosed, e.g., in U.S. Pat. Nos. 5,324,519; 4,938,763; 5,702,716; 5,744,153; and 5,990,194; wherein the suitable biodegradable thermoplastic polyester is disclosed as a thermoplastic polymer. Examples of suitable biodegradable thermoplastic polyesters include polylactides, polyglycolides, polycaprolactones, copolymers thereof, terpolymers thereof, and any combinations thereof. In some such embodiments, the suitable biodegradable thermoplastic polyester is a polylactide, a polyglycolide, a copolymer thereof, a terpolymer thereof, or any combination thereof. In one embodiment, the biodegradable thermoplastic polyester is 50/50 poly(DL-lactide-co-glycolide) having a carboxy terminal group; is present in about 30 wt. % to about 40 wt. % of the formulation; and has an average molecular weight of about 23,000 to about 45,000. Alternatively, in another embodiment, the biodegradable thermoplastic polyester is 75/25 poly (DL-lactide-co-glycolide) without a carboxy terminal group; is present in about 40 wt. % to about 50 wt. % of the formulation; and has an average molecular weight of about 15,000 to about 24,000. In further or alternative embodiments, the terminal groups of the poly(DL-lactide-co-glycolide) are either hydroxyl, carboxyl, or ester depending upon the method of polymerization. Polycondensation of lactic or glycolic acid provides a polymer with terminal hydroxyl and carboxyl groups. Ring-opening polymerization of the cyclic lactide or glycolide monomers with water, lactic acid, or glycolic acid provides polymers with the same terminal groups. However, ring-opening of the cyclic monomers with a monofunctional alcohol such as methanol, ethanol, or 1-dodecanol provides a polymer with one hydroxyl group and one ester terminal groups. Ring-opening polymerization of the cyclic monomers with a diol such as 1,6-hexanediol or polyethylene glycol provides a polymer with only hydroxyl terminal groups.

Since the polymer systems of thermoreversible gels dissolve more completely at reduced temperatures, methods of solubilization include adding the required amount of polymer to the amount of water to be used at reduced temperatures. Generally after wetting the polymer by shaking, the mixture is capped and placed in a cold chamber or in a thermostatic container at about 0-10° C. in order to dissolve the polymer. The mixture is stirred or shaken to bring about a more rapid dissolution of the thermoreversible gel polymer. The active agent and various additives such as buffers, salts, and preservatives are subsequently added and dissolved. In some instances the active agent and/or other pharmaceutically active agent is suspended if it is insoluble in water. The pH is modulated by the addition of appropriate buffering agents. Round window membrane mucoadhesive characteristics are optionally imparted to a thermoreversible gel by incorporation of round window membrane mucoadhesive carbomers, such as Carbopol® 934P, to the formulation (Majithiya et al., AAPS PharmSciTech (2006), 7(3), p. El; EP0551626, both of which is incorporated herein by reference for such disclosure).

In one embodiment are auris-acceptable pharmaceutical gel formulations which do not require the use of an added viscosity enhancing agent or viscosity modulating agent. Such gel formulations incorporate at least one pharmaceutically acceptable buffer. In one aspect is a gel formulation and a pharmaceutically acceptable buffer. In another embodiment, the pharmaceutically acceptable excipient or carrier is a gelling agent.

Also described herein are controlled-release formulations or devices a viscosity enhancing agent or viscosity modulating agent. Suitable viscosity-enhancing agents or viscosity modulating agents include by way of example only, gelling agents and suspending agents. In one embodiment, the enhanced viscosity formulation does not include a buffer. In other embodiments, the enhanced viscosity formulation includes a pharmaceutically acceptable buffer. Sodium chloride or other tonicity agents are optionally used to adjust tonicity, if necessary.

By way of example only, the auris-acceptable viscosity agent includes hydroxypropyl methylcellulose, hydroxyethyl cellulose, polyvinylpyrrolidone, carboxymethyl cellulose, polyvinyl alcohol, sodium chondroitin sulfate, sodium hyaluronate. Other viscosity enhancing agents compatible with the targeted auris structure include, but are not limited to, acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or combinations thereof. In specific embodiments, the viscosity-enhancing excipient is a combination of MCC and CMC. In another embodiment, the viscosity-enhancing agent is a combination of carboxymethylated chitosan, or chitin, and alginate. The combination of chitin and alginate with the active agent disclosed herein acts as a controlled-release formulation, restricting the diffusion of the active agent from the formulation. Moreover, the combination of carboxymethylated chitosan and alginate is optionally used to assist in increasing the permeability of the active agent through the round window membrane.

In some embodiments is an enhanced viscosity formulation, comprising from about 0.1 mM and about 100 mM of an active agent, a pharmaceutically acceptable viscosity enhancer or viscosity modulating agent, and water for injection, the concentration of the viscosity enhancer or viscosity modulating agent in the water being sufficient to provide an enhanced viscosity formulation with a final viscosity from about 100 to about 100,000 cP. In certain embodiments, the viscosity of the gel is in the range from about 100 to about 50,000 cP, about 100 cP to about 1,000 cP, about 500 cP to about 1500 cP, about 1000 cP to about 3000 cP, about 2000 cP to about 8,000 cP, about 4,000 cP to about 50,000 cP, about 10,000 cP to about 500,000 cP, about 15,000 cP to about 1,000,000 cP. In certain embodiments, the viscosity of the gel is in the range from about 100 to about 50,000 cP, about 100 cP to about 1,000 cP, abotlit 500 cP to about 1500 cP, about 1000 cP to about 3000 cP, about 2000 cP to about 8,000 cP, about 4,000 cP to about 50,000 cP, about 10,000 cP to about 500,000 cP, about 15,000 cP to about 3,000,000 cP. In other embodiments, when an even more viscous medium is desired, the biocompatible gel comprises at least about 35%, at least about 45%, at least about 55%, at least about 65%, at least about 70%, at least about 75%, or even at least about 80% or so by weight of the active agent. In highly concentrated samples, the biocompatible enhanced viscosity formulation comprises at least about 25%, at least about 35%, at least about 45%, at least about 55%, at least about 65%, at least about 75%, at least about 85%, at least about 90%, at least about 95%, or more by weight of the active agent.

In some embodiments, the viscosity of the gel formulations presented herein are measured by any means described. For example, in some embodiments, an LVDV-II+CP Cone Plate Viscometer and a Cone Spindle CPE-40 is used to calculate the viscosity of the gel formulation described herein. In other embodiments, a Brookfield (spindle and cup) viscometer is used to calculate the viscosity of the gel formulation described herein. In some embodiments, the viscosity ranges referred to herein are measured at room temperature. In other embodiments, the viscosity ranges referred to herein are measured at body temperature (e.g., at the average body temperature of a healthy human).

In one embodiment, the pharmaceutically acceptable enhanced viscosity auris-acceptable formulation comprises at least one active agent and at least one gelling agent. Suitable gelling agents for use in preparation of the gel formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum, and any combinations or mixtures thereof. In some other embodiments, hydroxypropylmethylcellulose (Methocel®) is utilized as the gelling agent. In certain embodiments, the viscosity enhancing agents or viscosity modulating agents described herein are also utilized as the gelling agent for the gel formulations presented herein.

In one specific embodiment of the auris-acceptable controlled-release formulations described herein, the active agent is provided in a gel matrix, also referred to herein as “auris-acceptable gel formulations”, “auris interna-acceptable gel formulations”, “auris media-acceptable gel formulations”, “auris externa-acceptable gel formulations”, “auris gel formulations”, or variations thereof. All of the components of the gel formulation must be compatible with the targeted auris structure. Further, the gel formulations provide controlled-release of the active agent to the desired site within the targeted auris structure; in some embodiments, the gel formulation also has an immediate or rapid release component for delivery of the active agent to the desired target site. In other embodiments, the gel formulation has a sustained release component for delivery of the active agent. In some embodiments, the auris gel formulations are biodegradable. In other embodiments, the auris gel formulations include a mucoadhesive excipient to allow adhesion to the external mucous layer of the round window membrane. In yet other embodiments, the auris gel formulations include a penetration enhancer excipient; in further embodiments, the auris gel formulation contains a viscosity enhancing agent sufficient to provide a viscosity of from about 10 to about 1,000,000 centipoise, from about 500 and 1,000,000 centipoise; from about 750 to about 1,000,000 centipoise; from about 1000 to about 1,000,000 centipoise; from about 1000 to about 400,000 centipoise; from about 2000 to about 100,000 centipoise; from about 3000 to about 50,000 centipoise; from about 4000 to about 25,000 centipoise; from about 5000 to about 20,000 centipoise; or from about 6000 to about 15,000 centipoise. In some embodiments, the auris gel formulation contains a viscosity enhancing agent sufficient to provide a viscosity of from about 50,0000 to about 1,000,000 centipoise. In some embodiments, the auris gel formulation contains a viscosity enhancing agent sufficient to provide a viscosity of from about 50,0000 to about 3,000,000 centipoise.

### Triglyceride Based Otic Formulations and Compositions

Provided herein in one embodiment are otic formulations and compositions comprising triglycerides. Triglycerides are esters derived from glycerol and three fatty acids. In some instances, these fatty acids are saturated fatty acids, unsaturated fatty acids, or a combination thereof. Provided herein in one aspect, is an otic formulation or a composition comprising a therapeutic agent, or pharmaceutically acceptable prodrug or salt thereof; and triglycerides comprising medium chain fatty acids; wherein the triglycerides are present in an amount that is sufficient to stabilize the therapeutic agent for injection into the ear, and wherein the otic pharmaceutical formulation or composition comprises at least about 50% by weight of the triglycerides.

In some instances, these triglycerides are medium chain triglycerides (MCTs). In some embodiments, these triglycerides comprise medium chain fatty acids. In some embodiments, these triglycerides are derived from glycerol and medium-chain fatty acids. In some embodiments, these triglycerides are derived from glycerol and at least two medium-chain fatty acids. In some embodiments, these triglycerides are derived from glycerol, two medium-chain fatty acids, and one long-chain fatty acid. In some embodiments, these triglycerides are derived from glycerol, and three medium-chain fatty acids.

In some embodiments, the triglycerides are derived from glycerol and medium chain fatty acids. In some embodiments, the triglycerides are derived from glycerol and at least two medium-chain fatty acids. In some embodiments, each medium chain fatty acid independently comprises 6 to 12 carbon atoms in the carbon chain. In some embodiments, each medium chain fatty acid independently comprises 8 to 12 carbon atoms in the carbon chain. In some embodiments, each medium chain fatty acid independently comprises 6, 7, 8, 9, 10, 11, or 12 carbon atoms in the carbon chain. In some embodiments, each medium chain fatty acid independently comprises 8 or 10 carbon atoms in the carbon chain. In some embodiments, the medium chain fatty acids are caproic acid (hexanoic acid), enanthic acid (heptanoic acid), caprylic acid (octanoic acid), pelargonic acid (nonanoic acid), capric acid (decanoic acid), undecylenic acid (undec-10-enoic acid), lauric acid (dodecanoic acid), or a combination thereof. In some embodiments, the medium chain fatty acids are caprylic acid (octanoic acid), capric acid (decanoic acid), or a combination thereof.

In some embodiments, the triglycerides comprising medium chain fatty acids are balassee oil, coconut oil, cohune oil, palm kernel oil, tucum oil, or combinations thereof. In some embodiments, triglycerides comprising medium chain fatty acids are coconut oil, cohune oil, palm kernel oil, tucum oil, or any combinations thereof. In some embodiments, the triglycerides comprising medium chain fatty acids are balassee oil. In some embodiments, the triglycerides comprising medium chain fatty acids are coconut oil. In some embodiments, the triglycerides comprising medium chain fatty acids are cohune oil. In some embodiments, the triglycerides comprising medium chain fatty acids are palm kernel oil. In some embodiments, the triglycerides comprising medium chain fatty acids are tucum oil.

In some embodiments, the otic pharmaceutical formulation has triglycerides in an amount that is sufficient to stabilize the therapeutic agent for injection into the ear. In some embodiments, the otic pharmaceutical formulation has triglycerides in an amount that is sufficient to provide sufficient retention time in the ear. In some embodiments, the ear is the outer ear, middle ear, or inner ear. In some embodiments, the otic pharmaceutical formulation has triglycerides in an amount that is sufficient to provide sustained release of the therapeutic agent. In some embodiments, the otic formulation has triglycerides in an amount that is sufficient to allow delivery of the formulation via a narrow gauge needle.

In some embodiments, the otic pharmaceutical formulation comprises between about 50% to about 99.9% by weight of the triglycerides. In some embodiments, the otic pharmaceutical formulation comprises between about 55% to about 99.9% by weight of the triglycerides. In some embodiments, the otic pharmaceutical formulation comprises between about 60% to about 99.9% by weight of the triglycerides. In some embodiments, the otic pharmaceutical formulation comprises between about 65% to about 99.9% by weight of the triglycerides. In some embodiments, the otic pharmaceutical formulation comprises between about 70% to about 99.9% by weight of the triglycerides. In some embodiments, the otic pharmaceutical formulation comprises between about 75% to about 99.9% by weight of the triglycerides. In some embodiments, the otic pharmaceutical formulation comprises between about 80% to about 99.9% by the weight of the triglycerides. In some embodiments, the otic pharmaceutical formulation comprises between about 85% to about 99.9% by the weight of the triglycerides. In some embodiments, the otic pharmaceutical formulation comprises between about 90% to about 99.9% by the weight of the triglycerides. In some embodiments, the otic pharmaceutical formulation comprises between about 95% to about 99.9% by the weight of the triglycerides.

In some embodiments, the otic pharmaceutical formulation comprises between about 50% to about 99.99% by weight of the triglycerides. In some embodiments, the otic pharmaceutical formulation comprises between about 55% to about 99.99% by weight of the triglycerides. In some embodiments, the otic pharmaceutical formulation comprises between about 60% to about 99.99% by weight of the triglycerides. In some embodiments, the otic pharmaceutical formulation comprises between about 65% to about 99.99% by weight of the triglycerides. In some embodiments, the otic pharmaceutical formulation comprises between about 70% to about 99.99% by weight of the triglycerides. In some embodiments, the otic pharmaceutical formulation comprises between about 75% to about 99.99% by weight of the triglycerides. In some embodiments, the otic pharmaceutical formulation comprises between about 80% to about 99.99% by weight of the triglycerides. In some embodiments, the otic pharmaceutical formulation comprises between about 85% to about 99.99% by weight of the triglycerides. In some embodiments, the otic pharmaceutical formulation comprises between about 90% to about 99.99% by weight of the triglycerides. In some embodiments, the otic pharmaceutical formulation comprises between about 95% to about 99.99% by weight of the triglycerides.

In some embodiments, the otic pharmaceutical formulation comprises between about 50% to about 95% by weight of the triglycerides. In some embodiments, the otic pharmaceutical formulation comprises between about 55% to about 95% by weight of the triglycerides. In some embodiments, the otic pharmaceutical formulation comprises between about 60% to about 95% by weight of the triglycerides. In some embodiments, the otic pharmaceutical formulation comprises between about 65% to about 95% by weight of the triglycerides. In some embodiments, the otic pharmaceutical formulation comprises between about 70% to about 95% by weight of the triglycerides. In some embodiments, the otic pharmaceutical formulation comprises between about 75% to about 95% by weight of the triglycerides. In some embodiments, the otic pharmaceutical formulation comprises between about 80% to about 95% by weight of the triglycerides. In some embodiments, the otic pharmaceutical formulation comprises between about 85% to about 95% by weight of the triglycerides. In some embodiments, the otic pharmaceutical formulation comprises between about 90% to about 95% by weight of the triglycerides.

In some embodiments, the otic formulations or compositions described herein are suspension formulations or compositions. In some embodiments, the otic formulations or compositions described herein are solution formulations or compositions. In some embodiments, the otic formulations or compositions have greater viscosity than an aqueous liquid composition. In some embodiments, the formulation or composition has a viscosity of greater than 1 cP (centipoise). In some embodiments, the formulation or composition has a viscosity of at least about 10 cP, about 20 cP, about 30 cP, about 40 cP, about 50 cP, about 60 cP, about 70 cP, about 80 cP, about 90 cP, about 100 cP, about 200 cP, about 300 cP, about 400 cP, about 500 cP, about 600 cP, about 700 cP, about 800 cP, about 900 cP, about 1,000 cP, about 2,000 cP, about 3,000 cP, about 4,000 cP, about 5,000 cP, about 6,000 cP, about 7,000 cP, about 8,000 cP, about 9,000 cP, about 10,000 cP, about 15,000 cP, or about 20,000 cP. In some embodiments, the formulation or composition has a viscosity of less than about 1,000 cP. In some embodiments, the formulation or composition has a viscosity of less than about 10,000 cP. In some embodiments, the formulation or composition has a viscosity of about 2 cP to about 250,000 cP, about 2 cP to about 100,000 cP, about 2 cP to about 50,000 cP, about 2 cP to about 25,000 cP, about 2 cP to about 10,000 cP, about 2 cP to about 5,000 cP, about 2 cP to about 1,000 cP, about 2 cP to about 500 cP, about 2 cP to about 250 cP, about 2 cP to about 100 cP, about 2 cP to about 90 cP, about 2 cP to about 80 cP, about 2 cP to about 70 cP, about 2 cP to about 60 cP, about 2 cP to about 50 cP, about 2 cP to about 40 cP, about 2 cP to about 30 cP, about 2 cP to about 20 cP, or about 2 cP to about 10 cP. In some embodiments, the liquid formulation or composition has a viscosity of about 2 cP, about 5 cP, about 10 cP, about 20 cP, about 30 cP, about 40 cP, about 50 cP, about 60 cP, about 70 cP, about 80 cP, about 90 cP, about 100 cP, about 200 cP, about 300 cP, about 400 cP, about 500 cP, about 600 cP, about 700 cP, about 800 cP, about 900 cP, about 1,000 cP, about 5,000 cP, about 10,000 cP, about 20,000 cP, about 50,000 cP, about 100,000 cP, or about 250,000 cP.

In some embodiments, the otic composition or formulation is free or substantially free of water. In some embodiments, the otic composition or formulation comprises less than 10% by weight of water. In some embodiments, the otic composition or formulation comprises less than 9% by weight of water. In some embodiments, the otic composition or formulation comprises less than 8% by weight of water. In some embodiments, the otic composition or formulation comprises less than 7% by weight of water. In some embodiments, the otic composition or formulation comprises less than 6% by weight of water. In some embodiments, the otic composition or formulation comprises less than 5% by weight of water. In some embodiments, the otic composition or formulation comprises less than 4% by weight of water. In some embodiments, the otic composition or formulation comprises less than 3% by weight of water. In some embodiments, the otic composition or formulation comprises less than 2% by weight of water. In some embodiments, the otic composition or formulation comprises less than 1% by weight of water. In some embodiments, the otic composition or formulation comprises less than 0.5% by weight of water. In some embodiments, the otic composition or formulation comprises less than 0.1% by weight of water. In some embodiments, an otic composition or formulation disclosed herein comprises less than about 50 ppm of water. In some embodiments, an otic composition or formulation disclosed herein comprises less than about 25 ppm of water. In some embodiments, an otic composition or formulation disclosed herein comprises less than about 20 ppm of water. In some embodiments, an otic composition or formulation disclosed herein comprises less than about 10 ppm of water. In some embodiments, an otic composition or formulation disclosed herein comprises less than about 5 ppm of water. In some embodiments, an otic composition or formulation disclosed herein comprises less than about 1 ppm of water.

In some embodiments, the otic composition or formulation is free or substantially free of poloxamer. In some embodiments, the otic composition or formulation is free or substantially free of poloxamer 407. In some embodiments, the otic composition or formulation is free or substantially free of C1-C6 alcohols or C1-C6 glycols. In some embodiments, the otic composition or formulation is free or substantially free of C1-C4 alcohols or C1-C4 glycols.

By way of non-limiting example, the use of the following commonly used solvents should be limited, reduced or eliminated when formulating agents for administration to the ear: alcohols, propylene glycol, and cyclohexane. Thus, in some embodiments, an otic composition or formulation disclosed herein is free or substantially free of alcohols, propylene glycol, and cyclohexane. In some embodiments, an otic composition or formulation disclosed herein comprises less than about 50 ppm of each of alcohols, propylene glycol, and cyclohexane. In some embodiments, an otic composition or formulation disclosed herein comprises less than about 25 ppm of each of alcohols, propylene glycol, and cyclohexane. In some embodiments, an otic composition or formulation disclosed herein comprises less than about 20 ppm of each of alcohols, propylene glycol, and cyclohexane. In some embodiments, an otic composition or formulation disclosed herein comprises less than about 10 ppm of each of alcohols, propylene glycol, and cyclohexane. In some embodiments, an otic composition or formulation disclosed herein comprises less than about 5 ppm of each of alcohols, propylene glycol, and cyclohexane. In some embodiments, an otic composition or formulation disclosed herein comprises less than about 1 ppm of each of alcohols, propylene glycol, and cyclohexane.

In some embodiments, therapeutic agent, or pharmaceutically acceptable prodrug or salt thereof, is multiparticulate. In some embodiments, the therapeutic agent, or pharmaceutically acceptable prodrug or salt thereof, is essentially in the form of micronized particles. In some embodiments, the therapeutic agent, or pharmaceutically acceptable prodrug or salt thereof, is essentially dissolved in the otic pharmaceutical formulation or composition. Carriers

Suitable carriers for use in a formulation or composition described herein include, but are not limited to, any pharmaceutically acceptable solvent. For example, suitable solvents include polyalkylene glycols such as, but not limited to, polyethylene glycol (PEG) and any combinations or mixtures thereof. In other embodiments, the base is a combination of a pharmaceutically acceptable surfactant and solvent.

In some embodiments, other excipients include, sodium stearyl fumarate, diethanolamine cetyl sulfate, isostearate, polyethoxylated castor oil, benzalkonium chloride, nonoxyl 10, octoxynol 9, sodium lauryl sulfate, sorbitan esters (s.orbitan monolaurate, sorbitan monooleate, sorbitan monopalmitate, sorbitan monostearate, sorbitan sesquioleate, sorbitan trioleate, sorbitan tristearate, sorbitan laurate, sorbitan oleate, sorbitan palmitate, sorbitan stearate, sorbitan dioleate, sorbitan sesqui-isostearate, sorbitan sesquistearate, sorbitan tri-isostearate), lecithins, phospholipids, phosphatidyl cholines (c8-c18), phosphatidylethanolamines (c8-c18), phosphatidylglycerols (c8-c18), pharmaceutical acceptable salts thereof and combinations or mixtures thereof.

In further embodiments, the carrier is polyethylene glycol. Polyethylene glycol is available in many different grades having varying molecular weights. For example, polyethylene glycol is available as PEG 200; PEG 300; PEG 400; PEG 540 (blend); PEG 600; PEG 900; PEG 1000; PEG 1450; PEG 1540; PEG 2000; PEG 3000; PEG 3350; PEG 4000; PEG 4600, and PEG 8000. For purposes of the present disclosure, all grades of polyethylene glycol are contemplated for use in preparation of a formulation described herein. In some embodiments the polyethylene glycol used to prepare a formulation described herein is PEG 300.

In other embodiments, the carrier is a polysorbate. Polysorbates are nonionic surfactants of sorbitan esters. Polysorbates useful in the present disclosure include, but are not limited to polysorbate 20, polysorbate 40, polysorbate 60, polysorbate 80 (Tween 80) and any combinations or mixtures thereof. In further embodiments, polysorbate 80 is utilized as the pharmaceutically acceptable carrier.

In one embodiment, water-soluble glycerin-based auris-acceptable enhanced viscosity formulations utilized in the preparation of pharmaceutical delivery vehicles comprise at least one active agent containing at least about 0.1% of the water-soluble glycerin compound or more. In some embodiments, the percentage of active agent is varied between about 1% and about 95%, between about 5% and about 80%, between about 10% and about 60% or more of the weight or volume of the total pharmaceutical formulation. In some embodiments, the amount of the compound(s) in each therapeutically useful formulation is prepared in such a way that a suitable dosage will be obtained in any given unit dose of the compound. Factors such as solubility, bioavailability, biological half-life, route of administration, product shelf life, as well as other pharmacological considerations are contemplated herein.

If desired, the auris-acceptable pharmaceutical gels also contain co-solvents, preservatives, cosolvents, ionic strength and osmolality adjustors and other excipients in addition to buffering agents. Suitable auris-acceptable water soluble buffering agents are alkali or alkaline earth metal carbonates, phosphates, bicarbonates, citrates, borates, acetates, succinates and the like, such as sodium phosphate, citrate, borate, acetate, bicarbonate, carbonate, and tromethamine (TRIS). These agents are present in amounts sufficient to maintain the pH of the system at 7.4±0.2 and preferably, 7.4. As such, the buffering agent is as much as 5% on a weight basis of the total formulation.

Cosolvents are used to enhance the active agent solubility, however, some active agents are insoluble. These are often suspended in the polymer vehicle with the aid of suitable suspending or viscosity enhancing agents.

Moreover, some pharmaceutical excipients, diluents or carriers are potentially ototoxic. For example, benzalkonium chloride, a common preservative, is ototoxic and therefore potentially harmful if introduced into the vestibular or cochlear structures. In formulating a controlled-release formulation, it is adVised to avoid or combine the appropriate excipients, diluents or carriers to lessen or eliminate potential ototoxic components from the formulation, or to decrease the amount of such excipients, diluents, or carriers. Optionally, a controlled-release formulation includes otoprotective agents, such as antioxidants, alpha lipoic acid, calcium, fosfomycin or iron chelators, to counteract potential ototoxic effects that may arise from the use of specific therapeutic agents or excipients, diluents, or carriers.

### pH and Osmolarity

Described herein are otic formulations or compositions with an ionic balance that is compatible with the perilymph and/or the endolymph and does not cause any change in cochlear potential. In specific embodiments, osmolarity/osmolality of the present formulations or compositions is adjusted, for example, by the use of appropriate salt concentrations (e.g., concentration of sodium salts) or the use of tonicity agents which renders the formulations or compositions endolymph-compatible and/or perilymph compatible (i.e. isotonic with the endolymph and/or perilymph). In some instances, the endolymph-compatible and/or perilymph-compatible formulations or compositions described herein cause minimal disturbance to the environment of the inner ear and cause minimum discomfort (e.g., vertigo) to a mammal (e.g., a human) upon administration. In some embodiments, the formulations or compositions described herein are free of preservatives and cause minimal disturbance (e.g., change in pH or osmolarity, irritation) in auditory structures. In some embodiments, the formulations or compositions described herein comprise antioxidants that are non-irritating and/or non-toxic to otic structures.

As used herein, “practical osmolarity” means the osmolarity of a formulation that is measured by including the active agent and all excipients except the gelling and/or the thickening agent (e.g., polyoxyethylene-polyoxypropylene copolymers, carboxymethylcellulose or the like). The practical osmolarity of a formulation described herein is measured by any suitable method, e.g., a freezing point depression method as described in Viegas et. al., Int. J. Pharm., 1998, 160, 157-162. In some instances, the practical osmolarity of a formulation described herein is measured by vapor pressure osmometry (e.g., vapor pressure depression method) that allows for determination of the osmolarity of a formulation at higher temperatures. In some instances, vapor pressure depression method allows for determination of the osmolarity of a formulation comprising a gelling agent (e.g., a thermoreversible polymer) at a higher temperature wherein the gelling agent is in the form of a gel. In some embodiments, the practical osmolality of an otic formulation described herein is from about 100 mOsm/kg to about 1000 mOsrn/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, the formulations described herein have a practical osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L.

In some embodiments, the osmolarity at a target site of action (e.g., the perilymph) is about the same as the delivered osmolarity (i.e., osmolarity of materials that cross or penetrate the round window membrane) of any formulation described herein. In some embodiments, the formulations described herein have a deliverable osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L.

The main cation present in the endolymph is potassium. In addition the endolymph has a high concentration of positively charged amino acids. The main cation present in the perilymph is sodium: In certain instances, the ionic composition of the endolymph and perilymph regulate the electrochemical impulses of hair cells. In certain instances, any change in the ionic balance of the endolymph or perilymph results in a loss of hearing due to changes in the conduction of electrochemical impulses along otic hair cells. In some embodiments, a composition or formulation disclosed herein does not disrupt the ionic balance of the perilymph. In some embodiments, a composition or formulation disclosed herein has an ionic balance that is the same as or substantially the same as the perilymph. In some embodiments, a composition or formulation disclosed herein does not disrupt the ionic balance of the endolymph. In some embodiments, a composition or formulation disclosed herein has an ionic balance that is the same as or substantially the same as the endolymph. In some embodiments, a composition or formulation described herein is formulated to provide an ionic balance that is compatible with inner ear fluids (i.e., endolymph and/or perilymph).

The endolymph and the perilymph have a pH that is close to the physiological pH of blood. The endolymph has a pH range of about 7.2-7.9; the perilymph has a pH range of about 7.2-7.4. The in situ pH of the proximal endolymph is about 7.4 while the pH of distal endolymph is about 7.9.

In some embodiments, the pH of a formulation or composition described herein is adjusted (e.g., by use of a buffer) to an endolymph-compatible pH range of about 7.0 to 8.0, and a preferred pH range of about 7.2-7.9. In some embodiments, the pH of the formulations or compositions described herein is adjusted (e.g., by use of a buffer) to a perilymph—compatible pH of about 7.0-7.6, and a preferred pH range of about 7.2-7.4.

In some embodiments, useful formulations or compositions also include one or more pH adjusting agents or buffering agents. Suitable pH adjusting agents or buffers include, but are not limited to acetate, bicarbonate, ammonium chloride, citrate, phosphate, pharmaceutically acceptable salts thereof and combinations or mixtures thereof.

In a specific embodiment the pH of a formulation or composition described herein is between about 6.0 and about 7.6, between 7 and about 7.8, between about 7.0 and about 7.6, between about 7.2 and about 7.6, or between about 7.2 and about 7.4. In certain embodiments the pH of a formulation or composition described herein is about 6.0, about 6.5, about 7.0, about 7.1, about 7.2, about 7.3, about 7.4, about 7.5, or about 7.6. In some embodiments, the pH of any formulation or composition described herein is designed to be compatible with the targeted otic structure (e.g., endolymph, perilymph or the like).

In some embodiments, any formulation or composition described herein has a pH that allows for sterilization (e.g., by filtration or aseptic mixing or heat treatment and/or autoclaving (e.g., terminal sterilization)) of a formulation or composition without degradation of the therapeutic agent. In order to reduce hydrolysis and/or degradation of the therapeutic agent during sterilization, the buffer pH is designed to maintain pH of the formulation or composition, in the 7-8 range during the process of sterilization.

In specific embodiments, any formulation or composition described herein has a pH that allows for terminal sterilization (e.g., by heat treatment and/or autoclaving) of a formulation or composition without degradation of the therapeutic agent. For example, in order to reduce hydrolysis and/or degradation of the therapeutic agent during autoclaving, the buffer pH is designed to maintain pH of the formulation or composition in the 7-8 range at elevated temperatures. Any appropriate buffer is used depending on the therapeutic agent used in the formulation or composition. In some instances, since pKₐ of TRIS decreases as temperature increases at approximately −0.031° C. and pKₐ of PBS increases as temperature increases at approximately 0.0031° C., autoclaving at 250° F. (121° C.) results in a significant downward pH shift (i.e. more acidic) in the TRIS buffer whereas a relatively much less upward pH shift in the PBS buffer and therefore much increased hydrolysis and/or degradation of an otic agent in TRIS than in PBS. In some embodiments, degradation of a therapeutic agent is reduced by the use of an appropriate of a buffer as described herein.

In some embodiments, a pH of between about 6.0 and about 7.6, between about 7 and about 7.8, between about 7.0 and about 7.6, between about 7.2 and 7.6, between about 7.2 and about 7.4 is suitable for sterilization (e.g., by filtration or aseptic mixing or heat treatment and/or autoclaving (e.g., terminal sterilization)) of formulations or compositions described herein. In specific embodiments a formulation or composition pH of about 6.0, about 6.5, about 7.0, about 7.1, about 7.2, about 7.3, about 7.4, about 7.5, or about 7.6 is suitable for sterilization (e.g., by filtration or aseptic mixing or heat treatment and/or autoclaving (e.g., terminal sterilization)) of any formulation or composition described herein.

In general, the endolymph has a higher osmolality than the perilymph. For example, the endolymph has an osmolality of about 304 mOsm/kg H₂O while the perilymph has an osmolality of about 294 mOsm/kg H₂O. In some embodiments, formulations or compositions described herein are formulated to provide an osmolarity of about 250 to about 320 mM (osmolality of about 250 to about 320 mOsm/kg H₂O); and preferably about 270 to about 320 mM (osmolality of about 270 to about 320 mOsm/kg H₂O). In certain embodiments, tonicity agents are added to the formulations described herein in an amount as to provide a practical osmolality of an otic formulation of about 100 mOsm/kg to about 1000 mOsm/kg, from about 200 mOsm/kg to about 800 mOsm/kg, from about 250 mOsm/kg to about 500 mOsm/kg, from about 250 mOsm/kg to about 350 mOsm/kg, or from about 280 mOsm/kg to about 320 mOsm/kg. In some embodiments, the formulations described herein have a practical osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 280 mOsm/L to about 320 mOsm/L, or about 250 mOsm/L to about 320 mOsm/L.

In specific embodiments, osmolarity/osmolality of the present formulations or compositions is adjusted, for example, by the use of appropriate salt concentrations (e.g., concentration of potassium salts) or the use of tonicity agents which renders the formulations or compositions endolymph-compatible and/or perilymph-compatible (i.e. isotonic with the endolymph and/or perilymph. In some instances, the endolymph-compatible and/or perilymph-compatible formulations or compositions described herein cause minimal disturbance to the environment of the inner ear and cause minimum discomfort (e.g., vertigo and/or nausea) to a mammal upon administration.

In some embodiments, the deliverable osmolarity of any formulation described herein is designed to be isotonic with the targeted otic structure (e.g., endolymph, perilymph, or the like). In specific embodiments, auris formulations described herein are formulated to provide a delivered perilymph-suitable osmolarity at the target site of action of about 250 to about 320 mOsm/L and preferably about 270 to about 320 mOsm/L. In specific embodiments, auris formulations described herein are formulated to provide a delivered perilymph-suitable osmolality at the target site of action of about 250 to about 320 mOsm/kg H₂O or an osmolality of about 270 to about 320 mOsm/kg H₂O. In specific embodiments, the deliverable osmolarity/osmolality of the formulations (i.e., the osmolarity/osmolality of the formulation in the absence of gelling or thickening agents (e.g., thermoreversible gel polymers) is adjusted, for example, by the use of appropriate salt concentrations (e.g., concentration of potassium or sodium salts) or the use of tonicity agents which renders the formulations endolymph-compatible and/or perilymph-compatible (i.e. isotonic with the endolymph and/or perilymph) upon delivery at the target site. The osmolarity of a formulation comprising a thermoreversible gel polymer is an unreliable measure due to the association of varying amounts of water with the monomeric units of the polymer. The practical osmolarity of a formulation (i.e., osmolarity in the absence of a gelling or thickening agent (e.g. a thermoreversible gel polymer) is a reliable measure and is measured by any suitable method (e.g., freezing point depression method, vapor depression method). In some instances, the formulations described herein provide a deliverable osmolarity (e.g., at a target site (e.g., perilymph) that causes minimal disturbance to the environment of the inner ear and causes minimum discomfort (e.g., vertigo and/or nausea) to a mammal upon administration.

In some embodiments, any formulation or composition described herein is isotonic with the perilymph. Isotonic formulations or compositions are provided by the addition of a tonicity agent. Suitable tonicity agents include, but are not limited to any pharmaceutically acceptable sugar, salt or any combinations or mixtures thereof, such as, but not limited to dextrose, glycerin, mannitol, sorbitol, sodium chloride, and other electrolytes.

Useful otic formulations or compositions include one or more salts in an amount required to bring osmolality of the composition into an acceptable-range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite, and ammonium sulfate.

In further embodiments, the tonicity agents are present in an amount as to provide a final osmolality of an otic formulation or composition of about 100 mOsm/kg to about 500 mOsm/kg, from about 200 mOsm/kg to about 400 mOsm/kg, from about 250 mOsm/kg to about 350 mOkn/kg or from about 280 mOsm/kg to about 320 mOsm/kg. In some embodiments, the formulations or compositions described herein have a osmolarity of about 100 mOsm/L to about 500 mOsm/L, about 200 mOsm/L to about 400 mOsm/L, about 250 mOsm/L to about 350 mOsm/L, or about 280 mOsm/L to about 320 mOsm/L. In some embodiments, the osmolarity of any formulation or composition described herein is designed to be isotonic with the targeted otic structure (e.g., endolymph, perilymph or the like). In some embodiments, the formulations described herein have a pH and/or practical osmolarity as described herein, and have a concentration of active pharmaceutical ingredient from about 0.0001% to about 60%, from about 0.001% to about 40%, from about 0.01% to about 20%, from about 0.01% to about 10%, from about 0.01% to about 7.5%, from about 0.01% to about 6%, from about 0.01 to about 5%, from about 0.1 to about 10%, or from about 0.1 to about 6% of the active ingredient by weight of the formulation.

### Particle Size

In some embodment, the active agent is dissolved in the otic composition. In some embodiments, the active agent is suspended in the otic composition in the form of multiparticulates, micron-sized particles,.nanoparticles, and is optionally encapsulated or coated (e.g. with a polymeric material). Size reduction is used to increase surface area and/or modulate formulation dissolution properties. It is also used to maintain a consistent average particle size distribution (PSD) (e.g., micrometer-sized particles, nanometer-sized particles or the like) for any formulation or composition described herein. In some embodiments, the formulation or composition comprises micrometer-sized particles. In some embodiments, the formulation or composition comprises nanometer-sized particles. In some instances, any formulation or composition described herein comprises multiparticulates, i.e., a plurality of particle sizes (e.g., micronized particles, nano-sized particles, non-sized particles); i.e., the formulation or composition is a multiparticulate formulation or composition. In some embodiments, any formulation or composition described herein comprises one or more multiparticulate (e.g., micronized) therapeutic agents. Micronization is a process of reducing the average diameter of particles of a solid material. Micronized particles are from about micrometer-sized in diameter to about picometer —sized in diameter. In some embodiments, the use of multiparticulates (e.g., micronized particles) of a therapeutic agent, or an otic agent, allows for extended and/or sustained release of the therapeutic agent from any formulation described herein compared to a formulation or composition comprising non-multiparticulate (e.g., non-micronized) therapeutic agent. In some instances, formulations or compositions containing multiparticulate (e.g., micronized) therapeutic agents are ejected from a lmL syringe adapted with a 27G needle without any plugging or clogging. In some embodiments, the therapeutic agent is essentially in the form of micronized particles. In some embodiments, the therapeutic agent is essentially in the form of microsized particles. In some embodiments, the therapeutic agent is essentially in the form of nanosized particles.

In some embodiments, the particle size of the formulation or composition described herein increases the retention time of the formulation or composition described herein. In some embodiments, the particle size of the formulation or composition described herein provides slow release of the therapeutic agent. In some embodiments, the particle size of the formulation or composition described herein provides sustained release of the therapeutic agent. In some embodiments, the particle size is less than 450 nm, less than 400 nm, less than 350 nm, less than 300 nm, less than 275 nm, less than 250 nm, less than 225 nm, less than 200 nm in size, less than 175 nm, less than 150 nm, or less than 125 nm, or less than 100 nm. In some embodiments, the particle size is less than 300 nm. In some embodiments, the particle size is less than 250 nm. In some embodiments, the particle size is less than 200 nm.

In some instances, any particle in any formulation or composition described herein is a coated particle (e.g., a coated micronized particle) and/or a microsphere and/or a liposomal particle. Particle size reduction techniques include, by way of example, grinding, milling (e.g., air-attrition milling (jet milling), ball milling), coacervation, high pressure homogenization, spray drying and/or supercritical fluid crystallization. In some instances, particles are sized by mechanical impact (e.g., by hammer mills, ball mill and/or pin mills). In some instances, particles are sized via fluid energy (e.g., by spiral jet mills, loop jet mills, and/or fluidized bed jet mills). In some embodiments formulations described herein comprise crystalline particles. In some embodiments, formulations or compositions described herein comprise amorphous particles. In some embodiments, formulations or compositions described herein comprise therapeutic agent particles wherein the therapeutic agent is a free base, or a salt, or a prodrug of a therapeutic agent, or any combination thereof.

In some instances, a combination of a therapeutic agent and a salt of the therapeutic agent is used to prepare pulsed release otic formulations or compositions using the procedures described herein. In some formulations, a combination of a micronized therapeutic agent (and/or salt or prodrug thereof) and coated particles (e.g., nanoparticles, liposomes, microspheres) is used to prepare pulsed release otic formulations or compositions using any procedure described herein.

In some embodiments, a pulsed release profile is achieved by solubilizing up to 40% of the delivered dose of the therapeutic agent (e.g., micronized therapeutic agent, or free base or salt or prodrug thereof; multiparticulate therapeutic agent, or free base or salt or prodrug thereof) with the aid of cyclodextrins, surfactants (e.g., poloxamers (407, 338, 188), tween (80, 60, 20,81), PEG-hydrogenated castor oil, cosolvents like N-methyl-2-Pyrrolidone or the like and preparing pulsed release formulations or compositions using any procedure described herein.

In some specific embodiments, any otic formulation or composition described herein comprises one or more micronized therapeutic agents. In some of such embodiments, a micronized therapeutic agent comprises micronized particles, coated (e.g., with an extended release coat) micronized particles, or a combination thereof. In some of such embodiments, a micronized therapeutic agent comprising micronized particles, coated micronized particles, or a combination thereof, comprises a therapeutic agent as a free base, a salt, a prodrug or any combination thereof.

### Modes of Otic Administration

In some embodiments, the auris formulations or compositions described herein are administered into the ear canal, or in the vestibule of the ear. Access to, for example, the vestibular and cochlear apparatus occurs through the auris media including the round window membrane, the oval window/stapes footplate, the annular ligament and through the otic capsule/temporal bone. In some embodiments, otic administration of the formulations or compositions described herein avoids toxicity associated with systemic administration (e.g., hepatotoxicity, cardiotoxicity, gastrointestinal side effects, and renal toxicity) of the active agents. In some instances, localized administration in the ear allows an active agent to reach a target organ (e.g., inner ear) in the absence of systemic accumulation of the active agent. In some instances, local administration to the ear provides a higher therapeutic index for an active agent that otherwise have dose-limiting systemic toxicity.

Provided herein are modes of treatment for otic formulations or compositions that ameliorate or lessen otic disorders described herein. Drugs delivered to the inner ear have been administered systemically via oral, intravenous or intramuscular routes. However, systemic administration for pathologies local to the inner ear increases the likelihood of systemic toxicities and adverse side effects and creates a non-productive distribution of drug in which high levels of drug are found in the serum and correspondingly lower levels are found at the inner ear.

Provided herein are methods comprising the administration of said auris formulations or compositions on or near the round window membrane via intratympanic injection. In some embodiments, a composition disclosed herein is administered on or near the round window or the crista fenestrae cochleae through entry via a post-auricular incision and surgical manipulation into or near the round window or the crista fenestrae cochleae area. Alternatively, a formulation or composition disclosed herein is applied via syringe and needle, wherein the needle is inserted through the tympanic membrane and guided to the area of the round window or crista fenestrae cochleae. In some embodiments, a formulation or composition disclosed herein is then deposited on or near the round window or crista fenestrae cochleae for localized treatment. In other embodiments, a formulation or composition disclosed herein is applied via microcathethers implanted into the patient, and in yet further embodiments a composition disclosed herein is administered via a pump device onto or near the round window membrane. In still further embodiments, a formulation or composition disclosed herein is applied at or near the round window membrane via a microinjection device. In yet other embodiments, a formulation or composition disclosed herein is applied in the tympanic cavity. In some embodiments, a formulation or composition disclosed herein is applied on the tympanic membrane. In still other embodiments, a formulation or composition disclosed herein is applied onto or in the auditory canal. The formulations or compositions described herein, and modes of administration thereof, are also applicable to methods of direct instillation or perfusion of the inner ear compartments. Thus, the formulations or compositions described herein are useful in surgical procedures including, by way of non-limiting examples, cochleostomy, labyrinthotomy, mastoidectomy, stapedectomy, endolymphatic sacculotomy or the like.

### Intratympanic Injections

In some embodiments, a surgical microscope is used to visualize the tympanic membrane. In some embodiments, the tympanic membrane is anesthetized by any suitable method (e.g., use of phenol, lidocaine, and xylocaine). In some embodiments, the anterior-superior and posterior-inferior quadrants of the tympanic membrane are anesthetized.

In some embodiments, a puncture is made in the tympanic membrane to vent any gases behind the tympanic membrane. In some embodiments, a puncture is made in the anterior-superior quadrant of the tympanic membrane to vent any gases behind the tympanic membrane. In some embodiments, the puncture is made with a needle (e.g., a 25 gauge needle). In some embodiments, the puncture is made with a laser (e.g., a CO₂ laser). In one embodiment the delivery system is a syringe and needle apparatus that is capable of piercing the tympanic membrane and directly accessing the round window membrane or crista fenestrae cochleae of the auris interna.

In one embodiment, the needle is a hypodermic needle used for instant delivery of the formulation. The hypodermic needle is a single use needle or a disposable needle. In some embodiments, a syringe is used for delivery of the pharmaceutically acceptable otic agent-containing compositions as disclosed herein wherein the syringe has a press-fit (Luer) or twist-on (Luer-lock) fitting. In one embodiment, the syringe is a hypodermic syringe. In another embodiment, the syringe is made of plastic or glass. In yet another embodiment, the hypodermic syringe is a single use syringe. In a further embodiment, the glass syringe is capable of being sterilized. In yet a further embodiment, the sterilization occurs through an autoclave. In another embodiment, the syringe comprises a cylindrical syringe body wherein the formulation is stored before use. In other embodiments, the syringe comprises a cylindrical syringe body wherein the pharmaceutically acceptable otic formulations or compositions as disclosed herein is stored before use which conveniently allows for mixing with a suitable pharmaceutically acceptable buffer. In other embodiments, the syringe contains other excipients, stabilizers, suspending agents, diluents, or a combination thereof to stabilize or otherwise stably store the otic agent or other pharmaceutical compounds contained therein.

In some embodiments, the syringe comprises a cylindrical syringe body wherein the body is compartmentalized in that each compartment is able to store at least one component of the auris-acceptable otic formulation. In a further embodiment, the syringe having a compartmentalized body allows for mixing of the components prior to injection into the auris media or auris interna. In other embodiments, the delivery system comprises multiple syringes, each syringe of the multiple syringes contains at least one component of the formulation such that each component is pre-mixed prior to injection or is mixed subsequent to injection. In a further embodiment, the syringes disclosed herein comprise at least one reservoir wherein the at least one reservoir comprises an otic agent, or a pharmaceutically acceptable buffer, or a viscosity enhancing agent, or a combination thereof. Commercially available injection devices are optionally employed in their simplest form as ready-to-use plastic syringes with a syringe barrel, needle assembly with a needle, plunger with a plunger rod, and holding flange, to perform an intratympanic injection.

In some embodiments, a needle is used to deliver the formulations or compositions described herein. In some embodiments, a needle punctures the posterior-inferior quadrant of the tympanic membrane. In some embodiments, the needle is a standard gauge needle. In some embodiments, the needle is a narrow gauge needle. In some embodiments, the needle is wider than an 18 gauge needle. In another embodiment, the needle gauge is from about 18 gauge to about 30 gauge. In some embodiments, the needle gauge is from about 20 gauge to about 30 gauge. In some embodiments, the needle gauge is from about 25 gauge to about 30 gauge. In some embodiments, the needle gauge is about 18 gauge, about 19 gauge, about 20 gauge, about 21 gauge, about 22 gauge, about 23 gauge, about 24 gauge, about 25 gauge, about 26 gauge, about 27 gauge, about 28 gauge, about 29 gauge, or about 30 gauge. In a further embodiment, the needle is a 25 gauge needle. Depending upon the thickness or viscosity of a formulation or composition disclosed herein, the gauge level of the syringe or hypodermic needle is varied accordingly. In some embodiments, the formulations or compositions described herein are liquids and are administered via narrow gauge needles or cannulas (e.g., 22 gauge needle, 25 gauge needle, or cannula), minimizing damage to the tympanic membrane upon administration. The formulations or compositions described herein are administered with minimal discomfort to a patient.

In some embodiments, an otoendoscope (e.g., about 1.7 mm in diameter) is used to visualize the round window membrane. In some embodiments, any obstructions to the round window membrane (e.g., a false round window membrane, a fat plug, fibrous tissue) are removed.

In some embodiments, a formulation or composition disclosed herein is injected onto the round window membrane. In some embodiments, 0.1 to 0.5 cc of a formulation or composition disclosed herein is injected onto the round window membrane.

In some embodiments, the tympanic membrane puncture is left to heal spontaneously. In some embodiments, a paper patch myringoplasty is performed by a trained physician. In some embodiments, a tympanoplasty is performed by a trained physician. In some embodiments, an individual is advised to avoid water. In some embodiments, a cotton ball soaked in petroleum-jelly is utilized as a barrier to water and other environmental agents. Dosage

In some embodiments, auris formulations or compositions described herein are controlled release formulations, and are administered at reduced dosing frequency compared to the current standard of care. In certain instances, when an auris formulation or composition is administered via intratympanic injection, a reduced frequency of administration alleviates discomfort caused by multiple intratympanic injections in individuals undergoing treatment for a middle and/or inner ear disease, disorder or condition. In certain instances, a reduced frequency of administration of intratympanic injections reduces the risk of permanent damage (e.g., perforation) to the ear drum. In some embodiments, formulations or compositions described herein provide a constant, sustained, extended, delayed or pulsatile rate of release of an active agent into the inner ear environment and thus avoid any variability in drug exposure in treatment of otic disorders.

The formulations or compositions containing the compound(s) described herein are administered for prophylactic and/or therapeutic treatments. In therapeutic applications, the formulations or compositions are administered to a Patient already suffering from a disease, condition or disorder, in an amount sufficient to cure or at least partially arrest the symptoms of the disease, disorder or condition. Amounts effective for this use will depend on the severity and course of the disease, disorder or condition, previous therapy, the patients health status and response to the drugs, and the judgment of the treating physician.

The amount of a given agent that will correspond to such an amount will vary depending upon factors such as the particular compound, disease condition and its severity, but is nevertheless routinely determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-50 mg per administration, preferably 1-15 mg per administration. In some embodiments, the desired dose is conveniently presented in a single dose or as divided doses administered simultaneously (or over a short period of time) or at appropriate intervals.

In some embodiments, doses for otic formulations comprising a growth factor range from a low dose of about 0.05% (about 0.5 mg/ml), to a medium dose of about 0.15% (about 1.5 mg/ml), to a higher dose about 0.5% (about 5 mg/ml) of the growth factor. In some cases, the growth factor is BDNF. The remaindei of the formulation includes auris acceptable vehicle such as a thermoreversible gel and optional other components, as described herein. In some cases, preparing such doses requires a 50 mg/ml growth factor (e.g., BDNF) concentrate. Such a concentrate may have limited stability over time.

Clinical doses range from about 0.01% to about 0.25% (e.g., about 0.1 mg/ml to about 2.5 mg/ml) of growth factor (e.g., BDNF). In some cases, preparing such clinical doses (e.g., for use in a Phase 1 or Phase 2 clinical trial) may require a 10× growth factor concentrate of 1 mg/ml to 25 mg/ml.

Solubility and stability of growth factor formulations are evaluated for concentrates (e.g., BDNF concentrates) between 1 mg/ml to 15 mg/ml to determine the highest usable concentrations. Solubility of growth factor (e.g., BDNF) is assessed in three buffer systems, phosphate buffer (PB), phosphate buffered saline (PBS), and Tris, to determine concentration limits of active vial concentrates.

The injection volume for an otic formulation comprising a growth factor is about 0.2 ml.

### Frequency of Administration

In the case wherein the patient's condition does not improve, upon the doctor's discretion the administration of the compounds is administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds are given continuously; alternatively, the dose of drug being administered are temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday are from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. Patients, however, require intermittent treatment on a long-term basis upon any recurrence of symptoms in some embodiments.

In some embodiments, the initial administration is of a particular formulation and the subsequent administration is of a different formulation or active pharmaceutical ingredient.

### NON-LIMITING EMBODIMENTS

1. A method of treating hearing loss or hearing impairement in a human subject, comprising intratympanically administering an otic formulation to the human subject, wherein the otic composition comprises from about 0.005 mg to about 1.90 mg of brain-derived neurotrophic factor (BDNF) and an auris-acceptable vehicle, wherein the otic formulation is formulated to provide sustained release of BDNF into the inner ear.
2. The method of Embodiment 1, wherein the BDNF is a recombinant BDNF.
3. The method of Embodiment 1 or 2, wherein the composition comprises from 0.005 mg to 0.015 mg BDNF.
4. The method of Embodiment 3, wherein the composition comprises about 0.01 mg BDNF.
5. The method of Embodiment 1 or 2, wherein the composition comprises from 0.015 mg to 0.05 mg BDNF.
6. The method of Embodiment 5, wherein the composition comprises about 0.03 mg BDNF.
7. The method of Embodiment 1 or 2, wherein the composition comprises from 0.05 mg to 0.20 mg BDNF.
8. The method of Embodiment 7, wherein the composition comprises about 0.10 mg BDNF.
9. The method of Embodiment 1 or 2, wherein the composition comprises from 0.20 mg to 0.40 mg BDNF.
10. The method of Embodiment 9, wherein the composition comprises about 0.3 mg BDNF.
11. The method of Embodiment 1 or 2, wherein the composition comprises from 0.40 mg to 1.10 mg BDNF.
12. The method of Embodiment 9, wherein the composition comprises about 0.75 mg or about 0.78 mg BDNF.
13. The method of Embodiment 1-2, wherein the composition comprises from 1.10 mg to 1.90 mg BDNF.
14. The method of Embodiment 9, wherein the composition comprises about 1.5 mg or about 1.56 mg BDNF.
15. The method of any one of Embodiments 1-14, wherein the auris-acceptable vehicle is an auris-acceptable gel.
16. The method of Embodiment 15, wherein the auris-acceptable gel is a thermoreversible gel.
17. The method of Embodiment 15 or 16, wherein the auris-acceptable gel comprises a copolymer of polyoxyethylene and polyoxypropylene.
18. The method of Embodiment 17, wherein the copolymer of polyoxyethylene and polyoxypropylene is poloxamer 407.
19. The method of Embodiment 18, wherein the otic formulation comprises from about 14 wt % to about 18 wt % poloxamer 407.
20. The method of Embodiment 18, wherein the otic formulation comprises from about 15 wt % to about 17 wt % poloxamer 407.
21. The method of Embodiment 18, wherein the otic formulation comprises about 15.8 wt % or about 16 wt % poloxamer 407.
22. The method of any one of Embodiments 15-21, wherein the auris-acceptable gel has a gelation viscosity from about 15,000 cP and about 3,000,000 cP.
23. The method of any one of Embodiments 15-22, wherein the auris-acceptable gel is capable of being injected by a narrow gauge needle or cannula through the tympanic membrane.
24. The method of any one of Embodiments 15-23, wherein the otic formulation has an osmolarity from about 100 mOsm/L to about 1000 mOsm/L.
25. The method of any one of Embodiments 16-24, wherein the otic formulation has a gelation temperature from about 19° C. to about 42° C.
26. The method of any one of Embodiments 1-25, wherein the otic formulation has a pH from about 7.0 to about 8.0.
27. The method of any one of Embodiments 1-26, wherein the otic formulation is an aqueous formulation and essentially free of any non-aqueous solvents.
28. The method of any one of Embodiments 1-27, wherein the growth factor is dissolved in the otic formulation.
29. The method of any one of Embodiments 1-27, wherein the growth factor is suspended in the otic formulation.
30. The method of any one of Embodiments 1-29, wherein the otic formulation provides sustained release of BDNF into the inner ear over a period of at least 5 days.
31. The method of any one of Embodiments 1-29, wherein the otic formulation provides sustained release of BDNF into the inner ear over a period of at least 1 week.
32. The method of any one of Embodiments 1-29, wherein the otic formulation provides sustained release of BDNF into the inner ear over a period of at least 2 weeks.
33. The method of any one of Embodiments 1-29, wherein the otic formulation provides sustained release of BDNF into the inner ear over a period of at least 3 weeks.
34. The method of any one of Embodiments 1-29, wherein the otic formulation provides sustained release of BDNF into the inner ear over a period of at least 4 weeks.
35. The method of any one of Embodiments 1-34, wherein the otic formulation repairs ribbon synapses.
36. The method of any one of Embodiments 1-35, wherein the hearing loss or hearing impairement is selected from cochlear synaptopathy, hearing-in-noise difficulties, speech-in-noise hearing impairement, or combinations thereof.
37. The method of any one of Embodiments 1-36, wherein the hearing loss or hearing impairement is cochlear synaptopathy.
38. The method of any one of Embodiments 1-37, wherein the hearing loss or hearing impairement is hearing-in-noise difficulties.
39. The method of any one of Embodiments 1-38, wherein the hearing loss or hearing impairement is speech-in-noise hearing impairement.

### NON-LIMITING EXAMPLES

### Example 1

### Preparation of Otic Thermoreversible Gel Formulation Containing BDNF

TABLE 1
Thermoreversible Gel Growth Factor Otic Formulation
COMPOSITION A for Phase 1/2 Clinical Study

| Component | COMPOSITION A | |
|---|---|---|
| BDNF | 0.05-7.8 | mg/mL |
| Sodium Chloride | 0.5% | w/w |
| Poloxamer 407 | 15.8% | w/w |

| | |
|---|---|
| WFI (buffered to pH 7-8) | QS to 100% |

Characterization of Composition A is listed below:
pH: 7.0-8.0
Osmolality: 250-300 mOsm/kg
Gelation temperature: 20-30° C.
Density: approximately 1.03 g/mL

### Example 2

### Pharmacokinetic Study of COMPOSITION A in Rat Model

Inner ear pharmacokinetic of COMPOSITION A with various BDNF concentrations were studies using a rat model describes below.

BDNF in Poloxamer 407

Poloxamer 407 solution at 32% was prepared by slowly adding it to a cold buffer solution (50 mM tris buffer with saline, pH 7.4). Sterilization was achieved by filtration. Human recombinant BDNF was either diluted or concentrated with the same tris buffer solution to a concentration twice the target concentration. The solutions were sterilized by filtration. Combining the above two solutions at 1:1 ratio yields a solution or a suspension containing 16% P407 and a concentration range of 0.05 mg/mL to 5 mg/mL BDNF.

Pharmacokinetics

Female rats (Charles River) weighing 200-300 g of approximately 12-16 weeks of age served as subjects (N=4 per group). Prior to any procedures, animals were anesthetized using a combination of xylazine (10 mg/kg) and ketamine (90 mg/kg) for up to an hour via the intraperitoneal route. If needed, an intraoperative booster was administered intraperitoneally representing a one-tenth of the original dose.

Intratympanic injection—Each animal was positioned so that the head was tilted at an angle to favor injection towards the round window niche. Briefly, under visualization with an operating microscope, 20 1.1.L of the formulation was injected using a 25G (Gauge) 11/2 needle through the tympanic membrane into the superior posterior quadrant. Formulations were delivered using a perfusion pump at the rate of 2 μL/sec. Contact with the round window membrane was maintained for 30 minutes by placing the animal in a recumbent position. During the procedure and until recovery, animals were placed on a temperature controlled (40° C.) heating pad until consciousness was regained at which time they were returned to the vivarium.

Perilymph sampling procedure—The skin behind the ear of anesthetized rats was shaved and disinfected with povidone-iodine. An incision was then made behind the ear, and muscles were carefully retracted from over the bulla. A hole was drilled through the bulla using a dental burr so that the middle ear was exposed and accessed. The cochlea and the round window membrane were visualized under a stereo surgical microscope. The basal turn of bulla was cleaned by using small cotton ball. A unique microhole was hand drilled through the bony shell of the cochlea (cochlear capsule) adjacent to the round window. Perilymph (about 2μL) was then collected using a microcapillary inserted into the cochlear scala tympani. Perilymph samples were added to a vial containing 18 μL of acetonitrile/water (50/50, v/v), stored at −80° C. until analysis.

Cochlear epithelium sampling procedure—Under a dissection scope, the bulla was opened, and the cochlea exposed by removing all surrounding muscles, blood vessels and bone tissues. Holding the bulla, the cochlear wall bony structure was removed working from the base to the apex of the cochlea, exposing the organ of Corti. The exposed cochlear epithelium was gently exposed, separated from the underlying bone and transferred to a tube for analysis.

Analytical Method

Concentrations of BDNF in perilymph and cochlear epithelium samples were determined using commercially available ELISA kits. The limit of detection of human BDNF was 4 pg/mL in perilymph, and 40 ng/g in cochlear epithelium

Results

The inner ear PK of COMPOSITION A is summaried in FIG. 2, which shows sustained release of BDNF into the inner ear for at least one week, at least two weeks, at least three week or at least four weeks post intratympanic injection.

### Example 3

### Preclinical Toxicology Studies of BDNF in Poloxamer Formulations

Single-dose toxicology studies evaluated COMPOSITION A following administration using the intended clinical route, i.e., IT. The rat and cat were the primary rodent and non-rodent species used in the toxicological assessment, as they are particularly suitable for otological studies. As IT is considered a local route of administration, studies addressing the local tolerance of COMPOSITION A were conducted in guinea pigs and rats.

A summary of the toxicology studies is presented in Table 2

TABLE 2
Summary of Studies Assessing the Toxicology of Composition A

| Study Description | | | | | |
|---|---|---|---|---|---|
| Study #/Tabular Summary | Species | Duration | | Route | Form and Dose |
| Single Dose Toxicity | | | | | |
| Acute toxicity - Otic | Rat | 3 | months | IT | COMPOSITION A, up to 0.5% |
| Acute toxicity - Otic | Cat | 3 | months | IT | COMPOSITION A, up to 0.5% |
| Local Tolerance | | | | | |
| Dermal toxicity | Rat | 14 | days | Top. | COMPOSITION A, up to 0.5% |
| Other Studies | | | | | |
| Skin sensitization | GP | 4 | weeks | Top. | COMPOSITION A, up to 0.5% |

IT; intratympanic; Top: topical.

The main objectives of the studies were: to characterize the degree of toxicity in the otic compartment and systemically following IT injection of COMPOSITION A, and to characterize local tolerance and antigenicity (delayed contact hypersensitivity). The acute IT toxicity assessment of COMPOSITION A in rodent (rat) and non-rodent (cat) species revealed that a single COMPOSITION A administration had no adverse effects on otic endpoints (auditory function, middle ear histology, inner ear integrity) up to the highest tested dose of 0.5%. Therefore, NOAEL values were established at 0.5% COMPOSITION A in both rat and cat for otic endpoints.

Systemically, the IT administration of COMPOSITION A was not associated with any adverse changes that were deemed treatment-related in either rats or cats. In rats, a unique instance of microscopic findings was noted in the mammary gland of two out of five males at 0.5% COMPOSITION A but not in any other animals across treatment groups (Saline, vehicle P407, COMPOSITION A all doses) and time points. Mammary adenocarcinoma is a common background neoplasm that occurs as an aging change in Sprague-Dawley rats, and this likely represented a background change although a test article-effect could not be ruled out. In cats, a single occurrence of red focus/foci in the heart of a female at 0.5% COMPOSITION A was observed. No similar microscopic changes were observed in any other animals across treatment groups and time points, and, therefore, this was not considered test article related. The lack of findings is further supported by the fact that systemic exposure to BDNF administered intratympanically was negligible across a variety of tissues (plasma, brain, heart, kidney, liver and lung) at all COMPOSITION A doses. Therefore, NOAEL values were established at 0.5% COMPOSITION A in both rat and cat for systemic endpoints.

GLP studies were conducted to delineate the local tolerance and antigenicity profile of COMPOSITION A. COMPOSITION A, at the highest dose of 0.5% did not produce acute skin irritation following dermal application in rats and did not induce allergic contact dermatitis (delayed hypersensitivity) in guinea pigs. In all, COMPOSITION A is well tolerated up to and including the highest dose of 0.5% when applied locally. Overall, the toxicological assessments of COMPOSITION A and its active ingredient BDNF support its safe use for the locally administered (IT) treatment of patients with hearing impairments. Details of those toxicological assessments are provided below.

Acute Ototoxicity Following IT Administration of COMPOSITION A in Rats

In this GLP Study, the potential toxicity of COMPOSITION A was evaluated following acute administration via the intratympanic route in rats. Subjects received a single unilateral intratympanic injection at the round window membrane of 0.05%, 0.15% and 0.5% COMPOSITION A followed by a 2-week and 3-month recovery periods (n=10/gender/group), for a total duration of 3 months. An additional cohort with a 2-week recovery period for the purpose of functional observation battery evaluation and toxicokinetic assessment were also included. A single IT injection deposited 20 μL at the round window membrane, corresponding to doses of 10, 30 and 100 μg per ear. The treatment group assignments are described in Table 3.

TABLE 3
Treatment Group Assignments

| Group | | Recovery | | Number of Animals | |
|---|---|---|---|---|---|
| Number | Dose Level | Period | | Male | Female |
| 1 | Saline | 2 | weeks | 10 | 10 |
| 1 | Gentamicin | 2 | weeks | 10 | 10 |
| 3 | Poloxamer 407 Vehicle | 2 | weeks | 10 | 10 |
| 4 | 0.05% COMPOSITION | 2 | weeks | 10 | 10 |
| 5 | 0.15% COMPOSITION | 2 | weeks | 10 | 10 |
| 6 | 0.5% COMPOSITION A | 2 | weeks | 10 | 10 |
| 7 | Saline | 3 | months | 7 | 7 |
| 8 | Gentamicin | 3 | months | 6 | 7 |
| 9 | Poloxamer 407 Vehicle | 3 | months | 7 | 7 |
| 10 | 0.05% COMPOSITION | 3 | months | 7 | 7 |
| 11 | 0.15% COMPOSITION | 3 | months | 7 | 7 |
| 12 | 0.5% COMPOSITION A | 3 | months | 7 | 7 |
| 13 | Poloxamer 407 Vehicle | 2 | weeks | 5 | 5 |
| 14 | 0.05% COMPOSITION | 2 | weeks | 5 | 5 |
| 15 | 0.15% COMPOSITION | 2 | weeks | 5 | 5 |
| 16 | 0.5% COMPOSITION A | 2 | weeks | 5 | 5 |

Groups 1-6: The animals were distributed equally for cytocochleogram or middle/inner ear histopathology.
Groups 7-12: The animals were assigned to middle/inner ear histopathology.
Groups 13-16: The animals were assigned to Functional Observation Battery evaluation and toxicokinetic assessment (tissues collected: plasma, brain, heart, kidney, liver, lung).

Assessment Of ototoxicity was based on otoscopic examinations, auditory function using auditory brainstem response (ABR), as well as evaluations of the middle ear (histology) and inner ear (cytocochleogram). In addition, assessment of overall toxicity was based on mortality, clinical observations, body weight, physical examinations, as well as clinical and anatomic pathology.

Test article-related mortality was not observed over the course of this study. Two animals were noted to have died on study: Animal No. 8002 and 9005, from the gentamicin and P407 Vehicle dose groups, respectively. These animals died during ABR procedures and these are considered procedural/anesthetic-related deaths. There were no signs of adverse clinical observations in any of the treatment groups, including up to the highest 0.5% COMPOSITION A dose. Clinical pathology analysis revealed no test article related effects on hematology parameters or clinical chemistry parameters in either sex at any COMPOSITION A dose tested.

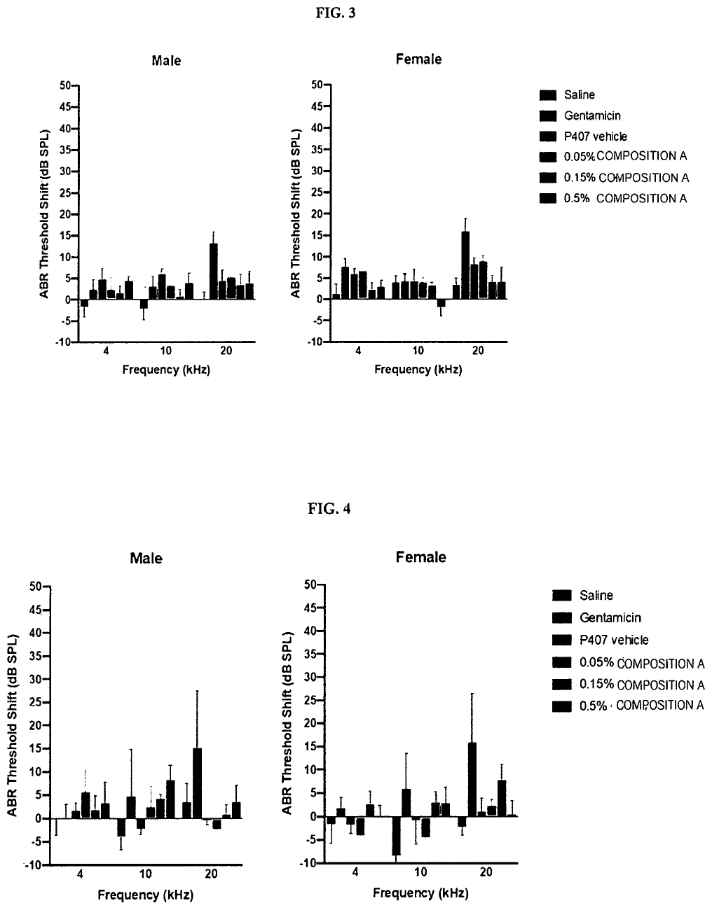

Hearing evaluation was conducted using ABR at three different frequencies (4, 10 and 20 kHz) at baseline, following the 2-week recovery period and at termination (3-month). Findings are presented in FIG. 3 (2-week recovery cohort) and FIG. 4 (3-month recovery cohort). The variability of the baseline ABR thresholds was similar between rats assigned to the different treatment groups at each tested frequency. In the saline group, no changes in ABR threshold were seen at either the 2-week or the 3-month termination endpoints in the treated ears. Similarly, in the P407 vehicle group, no changes in ABR threshold were evident at any frequency. Administration of the positive control, gentamicin (2900 mg/mL), a known ototoxicant, produced the expected threshold increases over the course of the study in both male and female rats. These changes are consistent with the hair cell losses observed in the basal and mid-regions of the organ of Corti. A single IT administration of COMPOSITION A, up to and including the highest dose of 0.5%, had no effects on ABR hearing thresholds at any frequency.

Macroscopic examination of the ears and organs revealed no definitive findings related to the administration of Saline, P407 vehicle or COMPOSITION A at any of the doses tested. One male at 0.5% COMPOSITION A (Animal No. 12001) in the 3-month cohort had a mass in the subcutis that correlated microscopically to a mammary adenocarcinoma. There were no additional macroscopic changes associated with mammary neoplasia. A potentially test article-related incidence of mammary gland neoplasia occurred in 2 out of 5 males at 0.5% COMPOSITION A. These changes were likely background due to the similarity to mammary gland neoplasia that occurs as a common aging change in rats of this strain but were not seen in other dose groups and a test article-effect could not be definitively excluded. A single intratympanic administration of COMPOSITION A up to and including the high dose of 0.5% followed by a 2-week or 3-month recovery period resulted in no test article-related changes to otic structures.

Microscopic changes in the middle ear in groups administered saline, vehicle, or test article were considered procedural and/or vehicle-related, irritant responses due to the introduction of foreign material to the middle ear, and/or background and not test article related. Gentamicin injection resulted in expected aminoglycoside associated changes to the inner ear as well as non-specific irritant responses. Cytocochleograms (assessment of inner ear integrity) were conducted at the 2-week recovery period. There were no adverse effects on hair cell integrity in the saline, P407 vehicle and OTO-413 treatment groups. Administration of gentamicin produced hair cell losses between 10 and 95% in the mid- and basal regions of the cochlea in all treated ears as expected. Untreated ears appeared normal in these animals. Therefore, with respect to both the auditory-related function and systemic effects of COMPOSITION A, the acute IT administration of COMPOSITION A up to and including 0.5% produced no adverse effects in rats. Overall, a no-observed-adverse-effect-level (NOAEL) of 0.5% COMPOSITION A was established for both otic and systemic endpoints.

Acute Ototoxicity Following IT Administration of COMPOSITION A in Cats

In this GLP Study, the potential toxicity of COMPOSITION A was evaluated following acute administration via the intratympanic route in cats. Subjects received a single unilateral intratympanic injection at the round window membrane of 0.05%, 0.15% and 0.5% COMPOSITION A followed by a 2-week and 3-month recovery periods (n=8/gender/group), for a total duration of 3 months. An additional cohort with a 2-week recovery period for toxicokinetic purposes was also included. A single IT injection deposited 150 !IL at the round window membrane, corresponding to doses of 75, 225 and 750 lig per ear. The treatment group assignments are described in Table 4 Assessment of ototoxicity was based on otoscopic examinations, auditory function using auditory brainstem response (ABR), as well as evaluations of the middle ear (histology) and inner ear (cytocochleogram). In addition, assessment of overall toxicity was based on mortality, clinical observations, body weight, physical examinations, as well as clinical and anatomic pathology.

TABLE 4
Treatment Group Assignments

| Group | | Recovery | | Number of Animals | |
|---|---|---|---|---|---|
| Number | Dose Level | Period | | Male | Female |
| 1 | Saline | 2 | weeks | 8 | 8 |
| 1 | Gentamicin | 2 | weeks | 8 | 8 |
| 3 | Poloxamer 407 Vehicle | 2 | weeks | 8 | 8 |
| 4 | 0.05% COMPOSITION | 2 | weeks | 8 | 8 |
| 5 | 0.15% COMPOSITION | 2 | weeks | 8 | 8 |
| 6 | 0.5% COMPOSITION A | 2 | weeks | 8 | 8 |
| 7 | Saline | 3 | months | 4 | 4 |
| 8 | Gentamicin | 3 | months | 4 | 4 |
| 9 | Poloxamer 407 Vehicle | 3 | months | 4 | 4 |
| 10 | 0.05% COMPOSITION | 3 | months | 4 | 4 |
| 11 | 0.15% COMPOSITION | 3 | months | 4 | 4 |
| 12 | 0.5% COMPOSITION A | 3 | months | 4 | 4 |
| 13 | Poloxamer 407 Vehicle | 2 | weeks | 4 | 4 |
| 14 | 0.05% COMPOSITION | 2 | weeks | 4 | 4 |
| 15 | 0.15% COMPOSITION | 2 | weeks | 4 | 4 |
| 16 | 0.5% COMPOSITION A | 2 | weeks | 4 | 4 |

Groups 1-6: The animals were distributed equally for cytocochleogram or middle/inner ear histopathology.
Groups 7-12: The animals were assigned to middle/inner ear histopathology.
Groups 13-16: The animals were assigned to toxicokinetic assessment (tissues collected: plasma, brain, heart, kidney, liver, lung).

No mortality was observed in any of the treatment groups, including up to the highest 0.5% COMPOSITION A dose. There were no signs of adverse clinical observations in any animals treated with saline, vehicle P407 or COMPOSITION A including up to the highest 0.5% COMPOSITION A dose. Gentamicin produced head tilt in most animals over the course of the study. This is a common finding in animals that have been administered this known ototoxicant. Clinical pathology analysis revealed no test article related effects on hematology parameters or clinical chemistry parameters in either sex at any COMPOSITION A dose tested.

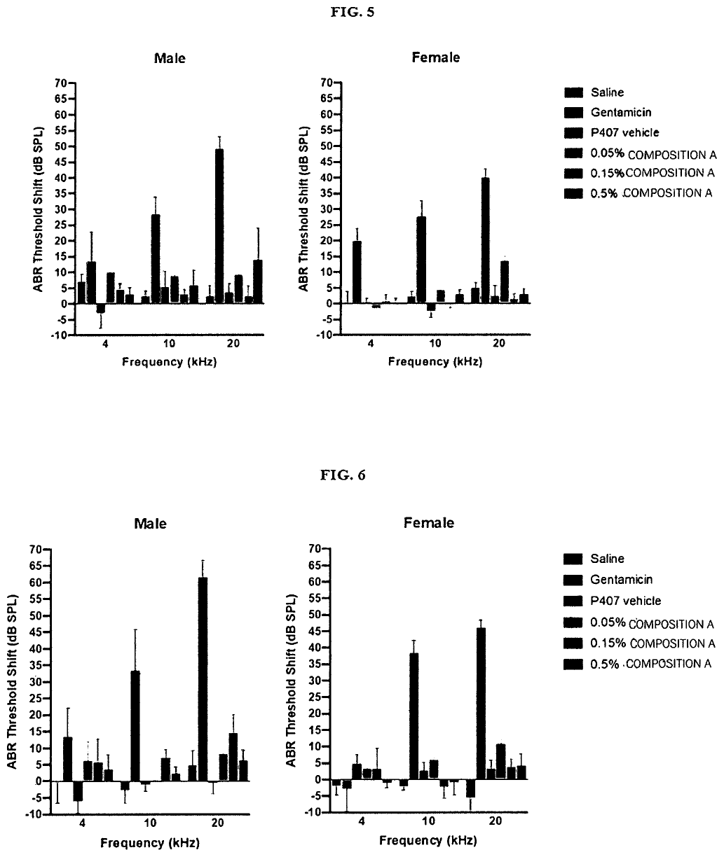

Hearing evaluation was conducted using ABR at three different frequencies (4, 10 and 20 kHz) at baseline, following the 2-week recovery period and at termination (3-month). Findings are presented in FIG. 5 (2-week recovery cohort) and FIG. 6 (3-month recovery cohort). The variability of the baseline ABR thresholds was similar between cats assigned to the different treatment groups at each tested frequency. In the saline group, no changes in ABR threshold were seen at either the 2-week or the 3-month termination endpoints in the treated ears.

Similarly, in the P407 vehicle group, no changes in ABR threshold were evident at any frequency. Administration of the positive control, gentamicin (1000 mg/mL) produced the expected threshold increases over the course of the study in both male and female cats. These changes are consistent with the hair cell losses observed in the basal and mid-regions of the organ of Corti. A single administration of COMPOSITION A, up to and including the highest dose of 0.5%, had no effects on ABR hearing thresholds.

Macroscopic examination of the ears and organs revealed no definitive findings related to the administration of Saline, P407 vehicle or COMPOSITION A at any of the doses tested. A single occurrence of red focus/foci in the heart of a female at 0.5% COMPOSITION A (Animal No. 6506) was considered an incidental background finding. This correlated microscopically to moderate multifocal myofiber degeneration/necrosis with minimal mineralization in the papillary muscle and cardiac myocytes near the endocardium. Based on this single finding the heart was evaluated microscopically for all animals across all treatment groups and time points and there was no similar microscopic finding in the heart of any other cat. There were no COMPOSITION A-related macroscopic findings at the 2-week or 3-month intervals.

A single intratympanic administration of COMPOSITION A up to and including the high dose of 0.5% followed by a 2-week or 3-month recovery period resulted in no test article-related changes to otic structures. Microscopic changes in the middle ear in groups administered saline, vehicle, or test article were considered procedural and/or vehicle-related irritant responses due to the introduction of foreign material to the middle ear, and/or background and not test article related. Vehicle-related changes were inflammatory in nature. Gentamicin injection resulted in expected aminoglycoside associated changes to the inner ear as well as non-specific irritant responses.

Cytocochleograms (assessment of inner ear integrity) were conducted at the 2-week recovery period. There were no adverse effects on hair cell integrity in the saline, P407 vehicle or OTO-413 treatment groups. Administration of gentamicin produced expected hair cell losses between 10 and 95% in the mid- and basal regions of the cochlea in all treated ears. Untreated ears appeared normal in these animals.

Therefore, with respect to both the auditory-related function and systemic effects of COMPOSITION A, the acute IT administration of COMPOSITION A up to and including 0.5% produced no adverse effects in cats. Overall, a no-observed-adverse-effect-level (NOAEL) of 0.5% COMPOSITION A was established for both otic and systemic endpoints.

Dermal Toxicity of COMPOSITION A in Rats

In this GLP Study, the potential of COMPOSITION A to cause dermal toxicity in rats (n=5/gender/group) was evaluated. This single dose acute dermal toxicity study was performed by topically administering (via a skin patch on the dorsal trunk, 1 mL) three doses of COMPOSITION A (0.05, 0.15 and 0.5% BDNF formulated in P407) for a contact period of 24 hours to male and female rats. The topical administration corresponds to applied doses of 0.5, 1.5 and 5 mg BDNF. Animals were observed daily for a period up to 2 weeks for clinical signs, including mortality, toxicity, pharmacological effects at 1 h and 4 h post-dosing, and once daily thereafter for 14 days. Dermal responses were scored at 24 h post-dosing and on Day 14.

COMPOSITION A did not produce mortality. Chromorhinorrhea and chromodacryorrhea were observed within 4 hours post-dosing and up to Day 1 among animals in all treatment groups. In the 0.5% COMPOSITION A group, one female appeared abnormal from Days 2-6, with evidence of coldness to the touch, piloerection, diminished fecal output, ataxia, tremors, wetness of the anogenital area and partially chewed food. Finally, COMPOSITION A up to and including the high dose of 0.5% did not produce acute skin irritation following dermal application.

Skin Sensitization of COMPOSITION A in Guinea Pigs

In this GLP Study, the potential of COMPOSITION A to cause or elicit skin sensitization reactions (allergic contact dermatitis) in guinea pigs was evaluated. This delayed hypersensitivity study was conducted in male and female guinea pigs (n=10 per sex). The experimental design, a skin sensitization study using the Buehler method, consisted of three phases: an induction phase, a transition period and a challenge phase.

The test article, vehicle and positive controls were administered topically to the dorsal flank three times over a period of 2 weeks using a Hilltop chamber (induction phase). Following a transition period of 2 weeks where animals were left untreated, a challenge exposure was performed (a unique application of the test article). Local reactions at the challenge site were reported at 24- and 48-hours following challenge using a sensitization scoring method based on the incidence, severity and duration of the sensitization phenotype.

A single dose of 0.5% COMPOSITION A (highest dose) was chosen based upon results from a range finding screen which demonstrated that COMPOSITION A doses of 0.05%, 0.15% and 0.5% did not produce dermal irritation. The total dose received by the 0.5% COMPOSITION A group was 8 mg. The vehicle control consisted of P407 vehicle administered during the induction phase but challenged with 0.5% COMPOSITION A. The positive control was hexyl cinnamic aldehyde (HCA), a known skin sensitizer.

Table 5 summarizes the sensitization scores for all groups. The positive control HCA produced an expected severity index of 0.55-0.70 classifying this control article as a sensitizer. The vehicle control (P407) did not induce sensitization over the course of the study and produced a SI of 0.00 (no reaction), classifying P407 as a non-sensitizer. COMPOSITION A, at the highest dose of 0.5%, did not induce sensitization over the course of the study and produced a SI of 0.00 (no reaction), classifying COMPOSITION A as a non-sensitizer.

TABLE 5
Delayed Hypersensitivity: Dermal Irritation
Scores of COMPOSITION A

Severity Index (SI)

| Test Article | 24 h | 48 h |
|---|---|---|
| P407 - Vehicle | 0.00 | 0.00 |
| HCA - Positive control | 0.70 | 0.55 |
| 0.5% COMPOSITION A | 0.00 | 0.00 |

The sensitization scoring was as follows: 0: no reaction; 0.5: very faint erythema, usually non-confluent; 1: obvious faint erythema, usually confluent; 2: moderate erythema; 3: strong erythema with or without edema.

### Example 4

### Speech-in-Noise Tests

A number of SIN tests have been used for clinical and research purposes. Examples include the Hearing-in-Noise Test (HINT; Nilsson et al., 1994), the Quick Speech-in-Noise test (QuickSIN; Killion et al., 2004), the Words-in-Noise test (WIN; Wilson, 2003), the Digits-in-Noise test (DIN; Watson et al., 2012), the American English Matrix test (AEMT; Kollmeier et al., 2015), Listening in Spatialized Noise-Sentences Test (LiSN-S: Cameron and Dillon, 2007), and Bamford-Kowal-Bench Speech-in-Noise test (BKB-SIN; Etymotic Research, Inc. 2005). In selecting tests for evaluating COMPOSITION A, we were interested in covering a range of test material modalities (e.g., digits, monosyllable words, and sentences) to determine which tests had sufficient test/re-test reliability and sensitivity for detecting SIN hearing changes. It was also important to utilize tests with minimal practice effects and/or to incorporate pre-testing to enable study subjects to become familiar with the test to limit learning effects. Finally, each test should have sufficient test material to enable repeat testing over an extended follow-up period; using the same speech material within a short time could heighten any learning effects as subjects may remember the word or sentence froM prior test sessions.

While digits-and monosyllable word tasks may more directly assess peripheral hearing than sentence-based tests which require more cognitive and linguistic input, sentence-based tests better represent everyday speech than digits or monosyllable words. Trends in performance on digits, words, and words in sentences are generally appreciated with increasing signal-to-noise ratios as the test items become more complex and the range of alternative possibilities increases (Miller et al., 1951). Using a battery of tests that utilize numbers, monosyllable words, and sentences enabled for evaluation across various SIN hearing modalities to comprehensively evaluate hearing under noisy conditions.

Digits-in-Noise Test (DIN)

Digits have been used in SIN testing for clinical diagnostic, screening, and research purposes (Wilson and Weakley 2004; van Wieringen and Wouters 2008; Smits 2004). Several different digits-in-noise tests have been developed and evaluated (see Van den Borre et al., 2011). Generally, the Digits-in-Noise test (DIN) employs a similar test paradigm with digit triplets (3 numbers between 0 and 9) presented in background masking noise (Smits et al., 2013). While the test was initially developed in Dutch, English versions have also been developed and validated (Watson et al., 2012; Smits et al., 2016). The advantage of a digits-based test is that it requires less linguistic or cognitive processing for comprehension compared to a more complex sentence-based test. Use of widely understandable digits enables a more focused assessment of peripheral auditory function. Another advantage is the DIN has limited practice effects meaning initial test results are typically similar to subsequent results for individual subjects. On the other hand, digits are limited in terms of phoneme distribution representative of daily life speech and do not approximate typical everyday listening conditions in which words are spoken in sentences or phrases.

The DIN we selected in our clinical study uses an adaptive one-up one-down procedure in which the signal-to-noise ratio (SNR) is automatically adjusted based on the previous response to determine the speech reception threshold (SRT; SNR at 50% correct for whole digit triplets). The SRT is calculated based on the average SNR across the last 20 of 23 digit-triplets administered. We focused on the 4 kHz low-pass filtered noise version of the DIN which has been demonstrated to have high sensitivity and specificity as well as a high level of test/re-test reliability (Motlagh Zadeh et al., 2019; Motlagh Zadeh et al., 2020).

Words-in-Noise Test (WIN)

The Words-in-Noise test (WIN) uses monosyllable words presented in multi-talker babble background noise (Wilson 2003; Wilson 2005). One advantage of the WIN is the use of single monosyllable words, which requires less central processing than multi-word sentences (but a greater central requirement than digits). Another key advantage is the use of multi-talker background noise of several speakers talking at the same time, a feature that approximates a real-world hearing environment that people typically encounter in a restaurant or social gathering. None of the background talking is intelligible and babble may impact speech to a greater degree than standard masking noise and therefore may be more relevant to everyday listening conditions (Wilson 2003).

The WIN uses the NU 6 monosyllable word materials recorded by a female speaker in the presence of multi-talker babble as the competing background noise. The 35-word version of the test was used in which groups of 5 words each were presented at 7 different, fixed signal-to-babble (S/B) ratios (Wilson 2005). We utilized 2 versions of the test with standard 70 dB HL sound presentation level as well as the more challenging 40 dB HL sound presentation level. The less traditional presentation level of 40 dB HL was chosen to challenge the listener further and potentially to increase the sensitivity for detecting SIN hearing changes. The 50% SRTs were determined using the Spearman-Karber equation as is standard for this test. The WIN has been demonstrated to have high test/re-test reliability following repeat assessments in the same individuals tested 1 to 3 months apart (Wilson 2007).

American English Matrix Test (AEMT)

The American English Matrix test (AEMT) is a sentence-based SIN test. The test uses S-word sentences presented in masking background noise. Each sentence is comprised of the same structure, i.e., name, verb, number, adjective, and noun. The sentences are grammatically correct, but semantically unpredictable (e.g., “Rachel has four pretty chairs”) thereby making them less likely to be correctly guessed if not heard properly. Another advantage of the AEMT is the large amount of test material available since the words for each sentence are randomly chosen from a base matrix containing 10 names, 10 verbs, 10 numbers, 10 adjectives, and 10 nouns which can generate thousands of different sentences. The large amount of test material facilitates repeat, longitudinal testing for individual subjects without the risk of subjects remembering a sentence from a prior test session. The matrix test is available in 14 different languages, although test performance differs by language (Kollmeier et al., 2015). The AEMT is administered adaptively and the SRT is determined by averaging the SNR at 50% correct performance for the 20 sentences of the test. The AEMT has high test/re-test reliability with a limited training effect (Zokoll et al., 2016), assuming practice testing was conducted beforehand.

Study Eligibility Criteria in Some Embodiments

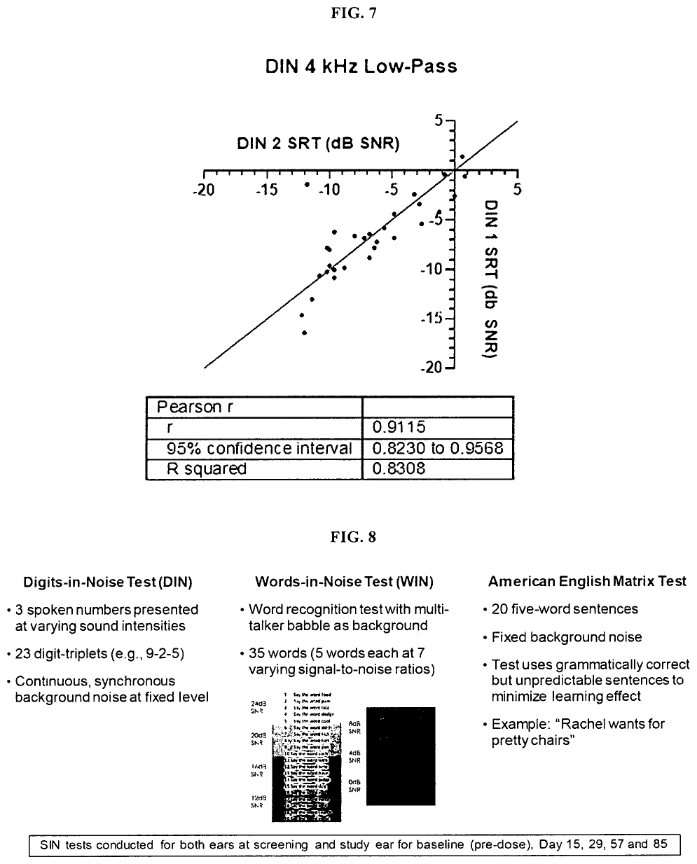

The ability of COMPOSITION A to improve SIN hearing is a key objective for clinical testing, so ensuring study subjects have a hearing-in-noise deficit is important for avoiding a ceiling effect. Therefore, to be eligible, subjects must self-report difficulty hearing in noisy environments for the preceding 6 months or longer to ensure the problem is present and persistent. In addition, a SIN deficit is confirmed objectively using a specific SIN test, in our case, we chose the DIN due to its advantages described above (e.g., high sensitivity, good test/re-test reliability, and minimal practice effects). The minimum score for eligibility (≥−12.5 dB SNR) was based on DIN SRT scores in individuals who subjectively complained of SIN hearing difficulty with either normal or mild hearing impairment via pure tone audiometry (Motlagh Zadeh et al., 2020). As illustrated in FIG. 7, there is a high degree of test/re-test reliability in subjects upon repeat testing approximately one week after the initial assessment based on DIN tests. FIG. 8 illustrate non-limiting examples of speech-in-noise tests used in certain embodiments disclosed herein.

Additional inclusion criteria for the study were pure tone audiometry thresholds for normal hearing or up to moderately severe hearing loss (≤70 dB of the average at 1, 2, and 4 kHz) in the study ear. We hypothesized that subjects with a range of auditory thresholds could benefit as long as a SIN deficit was present, and as stated previously, COMPOSITION A through restoration of type I SGN synapses may benefit hearing comprehension in subjects ranging from those with normal audiograms to individuals with audiometric threshold changes indicative of moderate to severe hearing loss. Only native English speakers were included to ensure English fluency did not impact performance on the SIN tests which were all in English. Subjects had to be without cognitive impairment based on scores on the cognitive screening test, the Mini-Mental State Examination (Folstein et al., 1975). Professional musicians or those with significant formal musical training were excluded since these individuals often perform extremely well on SIN tests. Lastly, any subjects expected to be exposed to intense noise during the study were excluded.

Safety Assessments

Appropriate measures to monitor the safety and tolerability of COMPOSITION A following IT injection focus on standard clinical measures of safety as well as ear-specific measures. Although IT injection is now routine in otolaryngology/neurotology practice, the tympanic membrane and middle ear must be in a healthy state to consider an IT injection and hence, eligibility criteria are included to help ensure this. For example, subjects are excluded with active middle ear disease, abnormal or monomeric tympanic membrane, perforated tympanic membrane, or indwelling myringotomy tubes in the affected ear. Post-administration, otoscopic examinations are conducted in order to evaluate the appearance of the external ear canal and tympanic membrane as well as to record the presence and size of any tympanic membrane perforations; normally perforations are small in size (“pinhole”) and heal within a week or two after the injection. Tympanometry is performed to assess the function of the tympanic membrane and middle ear post-administration. Typically, perforations of the tympanic membrane are associated with abnormal tympanograms (e.g., Type B or C), which revert to normal (Type A), once the perforation heals. Lastly, pure tone audiometry is conducted to determine if there are any changes in hearing across a range of frequencies including extended high frequencies (250 to 12,500 Hz). In addition to these ear-specific assessments, general safety tests are also conducted including vital signs, clinical safety labs, and body weight in as well as monitoring for adverse events and any concomitant medications for treatment.

### Example 5

### Clinical Trial of BDNF in Poloxamer Formulations

In Example 2, single dose nonclinical toxicology studies in rats and cats have been conducted and the results support the clinical investigation of COMPOSITION A in the current study. Additionally, pharmacokinetic assessments have been conducted in these species that provides a complete profile of exposure of COMPOSITION A to the inner ear compartment after intratympanic administration of COMPOSITION A, with negligible systemic exposure.

This example summarizes the first study of intratympanic administration of COMPOSITION A in humans. The starting dose level of COMPOSITION A of 0.01 mg (0.05 mg/mL) for intratympanic administration was chosen based on the No Observable Adverse Effect Level (NOAEL) in nonclinical toxicity studies. Based upon local exposure in the most sensitive species and normalization of the dose to perilymph volume, this starting dose provides a 250-fold safety margin. Ascending intratympanic dose levels of 0.03 mg (0.15 mg/mL), 0.10 mg (0.5 mg/mL), and 0.30 mg (1.5 mg/mL) which provide safety margins of 83-fold, 25-fold, and 8.3-fold respectively, are planned to be administered sequentially once the safety and tolerability has been established for the prior dose level. Details of the clinical trial protocol are provided below.

### 1. Objectives

### 1.1. Primary Objective

### All Cohorts

To evaluate the safety and tolerability of intratympanic COMPOSITION A.

### 1.2. Secondary Objective

### Cohorts 1-4

To evaluate the exploratory efficacy of COMPOSITION A on electrophysiological measures of cochlear synaptopathy including auditory-evoked potentials and middle ear muscle reflex (MEMR).
To evaluate the exploratory efficacy of COMPOSITION A on speech-in-noise hearing measures using the Digits-in-Noise Test (DIN), Words-in-Noise Test (WIN), and American English Matrix Test (AEMT).
To evaluate the exploratory efficacy of COMPOSITION A on self-reported hearing using the short form of the Speech, Spatial and Qualities of Hearing Test (SSQ-12) and the Patient Global Impression of Change (PGIC).
To evaluate the exploratory efficacy of COMPOSITION A on hearing function using distortion product otoacoustic emissions (DPOAE) and high frequency audiometry.

### Cohorts 5-7

To evaluate the exploratory efficacy of COMPOSITION A on speech-in-noise hearing measures using the DIN, WIN, and AEMT and on self-reported hearing ability using the PGIC.

### 1.3. Other Objectives

### Cohorts 1-4 and Cohorts 6-7

To assess the plasma PK and immunogenicity (anti-drug antibodies) of COMPOSITION A. Cohort 5
To assess the immunogenicity (anti-drug antibodies) and if warranted plasma PK of COMPOSITION A.

### 2. Overview of Study Design

This is a randomized, double-blind, placebo-controlled, single ascending dose study to evaluate the safety, exploratory efficacy, plasma pharmacokinetics (PK) and immunogenicity of intratympanic injection of COMPOSITION A in subjects with speech-in-noise hearing deficits. Dosing is planned to be conducted in 4 escalating cohorts of 8 subjects each (N=32 subjects). Dosing is also planned for one expansion cohort (Cohort 5) for the 0.30 mg dose level and will consist of 30 subjects. Dosing is also planned for two additional escalating cohorts of 12 subjects each for Cohorts 6 and 7. The number of subjects may be higher if some subjects are replaced and/or if some cohort sizes are expanded to obtain further experience with some dose levels.
The duration for each subject will be up to 17 weeks, including an up to a 5-week Screening period, a single-injection, and a 12-week follow-up period.
After Screening in Cohorts 1 to 4, 8 eligible subjects for each escalating cohort will be randomly assigned to either COMPOSITION A (6 subjects) or placebo (2 subjects) for intratympanic injection to the study ear. For the expansion Cohort 5, 30 eligible subjects will be randomly assigned to either COMPOSITION A (20 subjects) or placebo (10 subjects) for a single intratympanic injection to the study ear. For each escalating Cohort 6 and 7, 12 eligible subjects per cohort will be randomly assigned to either COMPOSITION A (8 subjects) or placebo (4 subjects) for intratympanic injection to the study ear. While 3:1 (COMPOSITION A:placebo) is the planned randomization ratio for the initial Cohorts 1-4, the planned ratio will be to 2:1 (COMPOSITION A:placebo) for expansion Cohort 5 and for escalating Cohorts 6 and 7 to enable enrollment of additional placebo subjects. Subjects will undergo safety and exploratory efficacy testing for 12 weeks following the injection. Blood samples for plasma concentrations of BDNF will be obtained at Screening (pre-dose), and post-dose on Days 1 and 8 in escalation Cohorts 1-4 and Cohorts 6 and 7 (plasma concentrations may be determined for Cohort 5 if warranted). Blood samples will also be obtained for immunogenicity testing at Screening (pre-dose), and post-dose on Day 8, Day 29, and Day 85 for all cohorts (Cohorts 1-7).
For Cohorts 1-4 exploratory efficacy assessments will be collected throughout the study and include electrophysiological tests of cochlear synaptopathy (auditory-evoked potentials; middle ear muscle reflex [MEMR]), tests of speech-in-noise hearing ability (Digits-in-Noise Test [DIN], Words-in-Noise [WIN] Test, and American English Matrix Test [AEMT]), self-reported tests of hearing ability (the short form of the Speech, Spatial and Qualities of Hearing Scale questionnaire [SSQ-12]) and the Patient Global Impression of Change [PGIC]), and assessments of hearing function [DPOAE] and audiometry).
For Cohorts 5-7, exploratory efficacy assessments will be limited to tests of speech-in-noise hearing ability DIN, WIN, and AEMT, and a self-reported test of hearing ability (PGIC).
Approximately 94 total subjects will be enrolled according to the following cohort dose levels, total number of subjects, and randomization ratios:
Cohort 1: 0.01 mg, N=8 subjects (3:1 randomization ratio)
Cohort 2: 0.03 mg, N=8 subjects (3:1 randomization ratio)
Cohort 3: 0.10 mg, N=8 subjects (3:1 randomization ratio)
Cohort 4: 0.30 mg, N=8 subjects with expansion up to 16 subjects (3:1 randomization ratio)
Cohort 5: 0.30 mg, N=30 subjects (2:1 randomization ratio)
Cohort 6: 0.75 mg, N=12 subjects (2:1 randomization ratio)
Cohort 7: 1.50 mg, N=12 subjects (2:1 randomization ratio)

### Study Design Schematic

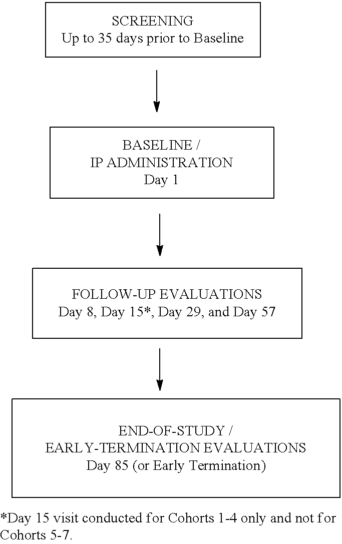

### 2.1. Dose Escalation and Safety Review Committee

Standard safety data reviews are conducted prior to escalating to the next higher dose-level cohort.

### 3. Study Population

### 3.1. General Considerations

Subjects will be eligible if they meet all of the following inclusion criteria and none of the exclusion criteria.

### 3.2. Inclusion Criteria

To be eligible for this study, each of the following criteria must be satisfied with a “YES” answer (unless not applicable):
1. Subject is a male or female aged 21 to 64 years, inclusive, at the time of informed consent.
2. Subject has audiometrically-defined normal hearing or up to moderately severe hearing impairment in at least one ear (study ear) as characterized by pure tone average of <70 dB at 1000, 2000, and 4000 Hz.
3. Subject has self-reported difficulty hearing in noisy environments for at least 6 months prior to Screening.
4. Subject exhibited a speech-in-noise hearing deficit in at least one ear (study ear) as indicated by the Digits-in-Noise test (e.g., ≥−12.5 dB signal-to-noise ratio [SNR]) at Screening.
5. Subject is a native English speaker (self-report) and is able to complete all study assessments including the speech-in-noise tests.
6. Subject has a score of ≥24 on the Mini-Mental State Examination.
7. Female subjects of childbearing potential [i.e., not surgically sterile (hysterectomy, bilateral oophorectomy or bilateral salpingectomy) and/or not post-menopausal (≥12 months since last menstrual period and 45 years of age or older)] must have a negative urine pregnancy test at Baseline. Women of childbearing potential who are not abstinent from sex with male partners must use effective methods of contraception for the duration of the study including: established use of oral, injected, or implanted hormonal methods of contraception; placement of an intrauterine device or intrauterine system; or “double barrier” methods including a combination of male condom with either diaphragm or cervical cap with spermicide. A vasectomized partner is also acceptable. Female subjects of childbearing potential must also refrain from egg donation or retrieval for the duration of the study.
8. Male subjects [unless surgically sterile (sterilized by vasectomy for at least 6 months with negative semen analysis)] who are not abstinent from sex with female partners of childbearing potential must agree to use an effective contraceptive method (as detailed for Inclusion Criteria 7) for the duration of the study. Male subjects must refrain from sperm donation for the duration of the study.
   Note: Abstinence (male or female subjects) is acceptable if this is the usual lifestyle and preferred contraception for the subject. Periodic abstinence, the rhythm method, and the withdrawal method are not acceptable.
9. Subject is willing to comply with the protocol and attend all study visits.
10. Subject is able to provide written informed consent after the scope and nature of the investigation have been explained, and before the initiation of any study-related procedures.

### 3.3. Main Criteria for Exclusion:

To be eligible for this study, each of the following criteria must be satisfied with a “NO” answer (unless not applicable):
1. Subject is pregnant or lactating.
2. Subject has the following hearing disorders or any other hearing disorders that may impact the efficacy assessments or safety of the subject in the opinion of the Investigator: Meniere's disease as defined by the American Academy of Otolaryngology-Head and Neck Surgery Equilibrium Committee in 2015 (Goebel 2016), congenital hearing loss, or genetic sensorineural hearing loss.
3. Subject has a cochlear implant or currently uses a hearing aid. Prior use of hearing aids is acceptable, but not within 7 days of the start of the Screening period.
4. Subject has worked at least 5 years as a professional musician or has had at least 15 years of formal musical training.
5. Subject has current (past 2 weeks prior to Screening) intense noise exposure due to either occupational, recreational, or other types of noise exposure OR is unable to avoid these types of noise exposure during study participation. Examples include: 1) occupational noise in professions requiring regular use of hearing protection, 2) target shooting/firing range exposure/military with exposure to weapons or loud vehicles, aircraft, or machinery, 3) hunting.
6. Subject is receiving any ongoing therapy known to affect hearing including but not limited to aminoglycosides, ototoxic chemotherapeutic agents (e.g., cisplatin), loop diuretics, quinine, high doses of aspirin or other nonsteroidal anti-inflammatory drugs. Usage of low doses of aspirin or other nonsteroidal anti-inflammatory drugs may be permitted at the Investigator's discretion.
7. Subject self-reports bothersome, subjective tinnitus and is consistently aware of their tinnitus throughout much of the waking day.
8. Subject has active middle ear disease (including but not limited to chronic otitis media, acute otitis media, middle ear effusions, middle ear atelectasis, or cholesteatoma) or vestibular schwannoma. .
9. Subject has an abnormality of the tympanic membrane in the study ear that would increase the risk associated with intratympanic injection, including but not limited to monomeric tympanic membrane.
10. Subject has evidence of perforation or lack of closure of the tympanic membrane in the study ear at Screening or Baseline.
11. Subject has a history of previous use of intratympanic gentamicin in either ear.
12. Subject has received intratympanic steroids (including use of dexamethasone) within 3 months prior to the Screening visit.
13. Subject has used an investigational drug or device in the 30 days prior to screening.
14. Subject has a history of substance or alcohol abuse within the preceding 6 months prior to Screening.
15. Subject has other clinically significant illness, medical condition or medical history at Screening or Baseline (Day 1) that, in the Investigator's opinion, would likely reduce the safety of study participation or compliance with study procedures.
16. Subject has previously participated in a clinical study with COMPOSITION A.

### 4. Randomization and Blinding

### 4.1. Overview

Four different escalating dose-level cohorts of COMPOSITION A (0.01 mg, 0.03 mg, 0.10 mg, and 0.30 mg), one expansion cohort of the COMPOSITION A 0.30 mg dose level, and two additional escalating dose-level cohorts (0.75 mg and 1.50 mg) are planned to be tested in this study. Eligible subjects in Cohorts 1-4 will be randomly assigned to either COMPOSITION A or placebo in a 3:1 allocation ratio (COMPOSITION A:placebo), based on a computer-generated randomization schedule. Expansion Cohort 5 (0.30 mg COMPOSITION A or placebo) and escalating Cohorts 6 and 7 will use a 2:1 (COMPOSITION A:placebo) randomization ratio.

### 4.2. Enrollment Procedures

### 4.2.1. Assignment of Subject Identification Numbers

At the Screening visit (Visit I), subjects signing the informed consent will be assigned a sequential subject identification number by the site. Once assigned, the subject identification number will not be re-assigned and should not be changed. This number will be used to identify the subject throughout the study.

### 4.2.2. Study Ear Assignment

COMPOSITION A or placebo is administered into a single ear by intratympanic injection. The ear to be injected, i.e., the study ear, is the ear with normal to moderately severe impairment via audiometric testing and speech-in-noise hearing impairment via the Digits-in-Noise test per the eligibility criteria at Screening. In the event that both ears meet the audiometric and speech-in-noise eligibility criteria at Screening, the most affected ear based on speech-in-noise testing will be the study ear. If the speech-in-noise test results are comparable for each ear, the most affected ear via audiometry will be selected as the study ear per the Investigator's judgment.

### 4.2.3. Treatment Assignment

After a subject has met all prerequisites for randomization on Day 1 (Baseline/Visit 2), study sites will execute each randomization via the interactive web randomization system (IWR). Once assigned, kit numbers cannot be re-assigned. Subjects will be considered enrolled into the study once they are randomized.
Study sites will provide the information contained in the IWR randomization notification to the person responsible for preparation of the syringe containing investigational product (COMPOSITION A or placebo). The unique kit number provided by IWR will correspond to a kit of packaged investigational product labeled with the identical kit number. The syringe will be prepared from the contents of the investigational product package corresponding to the kit number according to the instructions in the study Pharmacy Manual. The subject identification number and kit number both must be recorded in the subject's record.

### 4.2.4. Randomization Algorithm

Subjects will be randomized using either a 3:1 ratio (COMPOSITION A:placebo) for Cohorts 1-4 or a 2:1 ratio (COMPOSITION A:placebo) for Cohorts 5-7 using a permuted block randomization algorithm.
The randomization process will be deployed via IWR which is accessible 24 hours a day to authorized users. The subject's randomization number will determine the randomized treatment assignment. Investigational product kits will be labeled with a unique kit number using a separate and independent randomization algorithm. Numbered kits will be dispensed based on the treatment assignment.

### 4.3. Blinding

The study will be double-blinded. Each treatment syringe will be prepared by unblinded qualified research staff according to the detailed instructions in the Pharmacy Manual.
The blind should be broken for site personnel only if knowing the subject's treatment allocation would facilitate specific medical treatment. In all cases, the Investigator should consult with the medical monitor prior to unblinding, if possible, and must contact the medical monitor as soon as it is practical after unblinding has occurred.
If the blind is broken, the subject will continue to be followed and evaluated per-protocol. The date, time, and reason for the unblinding must be documented on the appropriate page of the eCRF.
The randomization schedule or blocking factor(s) will not be revealed to study subjects, Investigators, clinical staff, site managers or the Sponsor until all subjects have completed the study and the database has been finalized by the Sponsor.

### 5. Dosage and Administration

### 5.1. Investigational Product Administration

BDNF concentrate or placebo is provided in individual Investigational Product kits. The BDNF concentrate is stored at-20° C. until use, while the diluents and placebo are stored at 2-8° C. until use. Diluents, provided separately and not contained within the Investigational Product kit, are used to dilute the BDNF concentrate to the target concentration for each dose level (see Pharmacy Manual for instructions).
Syringes containing COMPOSITION A or placebo are prepared in a clean location at room temperature. Refer to the Pharmacy Manual for instructions on COMPOSITION A and placebo syringe preparation instructions.
COMPOSITION A or placebo will be administered as a single (0.2 mL volume) intratympanic injection to the study ear. Only an otolaryngologist may perform the intratympanic injection.
A 1 mL luer-lock sterile syringe should be used for intratympanic injection. Luer slip tip syringes are not acceptable for use due to the viscosity of COMPOSITION A. Recommended needles may be 25, 26, or 27 gauge and typically range from 1.5 to 3.5 inches in length.
The recommended injection procedure for intratympanic administration of COMPOSITION A or placebo in subjects is as follows. A ventilation hole in the tympanic membrane is not needed due to the small injection volume.
1. Place the subject in a recumbent position with the study ear upwards.
2. Prior to COMPOSITION A or placebo administration, confirm the ear to be treated is the study ear.
3. Anesthetize the tympanic membrane by covering the external surface of the inferior-posterior quadrant with a topical preparation containing lidocaine, prilocaine, or other related anesthetic (e.g., EMLA® cream or lidocaine spray or solution) until the tympanic membrane is numb. If necessary, suction away any excess topical preparation. Phenol should not be used to anesthetize the tympanic membrane.
4. Using the prepared syringe, insert the needle into the inferior-posterior quadrant of the tympanic membrane at the level of the round window, taking care not to insert the needle further than necessary.
5. With the needle bevel facing in the inferior-posterior direction, inject 0.2 mL of COMPOSITION A or placebo towards the round window.
5.2. Compliance
   COMPOSITION A or placebo will be administered by an otolaryngologist as a single, intratympanic injection at Day 1. Any deviations in administration will be documented in the source documents.
   The site will maintain a log of all investigational product dispensed and returned. Investigational product supplies for each subject will be inventoried and accounted for in the study.
6. Prior, Concomitant and Subsequent Therapy
   Use of all concomitant medications will be recorded in the subject's eCRF. This will include all prescription drugs, herbal products, vitamins, minerals, and over-the-counter medications taken within 30 days before randomization, which will be considered prior therapy. Any concomitant medication deemed necessary for the welfare of the subject during the study may be given at the discretion of the Investigator except for those medications listed in Section 6.1. Any changes in concomitant medications will be recorded in the subject's eCRF.

### 6.1. Prohibited Therapy During the Study Period

The following therapies are prohibited during the study:
Phenol for use in anesthetizing the tympanic membrane.
Intratympanic injection other than that outlined in the current study.
Other investigational drug(s) or device(s).
Agents that are known to affect hearing including but not limited to aminoglycosides, ototoxic chemotherapeutic agents (e.g., cisplatin), loop diuretics, quinine, high doses of aspirin or other nonsteroidal anti-inflammatory drugs. Usage of low doses of aspirin (i.e., daily doses of 81 mg) or low doses of other nonsteroidal anti-inflammatory drugs for intermittent pain relief may be permitted at the Investigator's discretion.
Hearing aid use is prohibited during the study period. However, past use of hearing aids is acceptable but not within 7 days of the start of the Screening period.
   Use of any of these prohibited therapies will be considered a protocol deviation.

### 7. Study Evaluations

### 7.1. Study Procedures by Visit

### 7.1.1. Screening Period: Up to 35 Days Prior to Baseline

For Cohorts 1-4, the following assessments, as listed in the Time and Events Schedule Error! Reference source not found., will be performed during the Screening period: informed consent, review and confirm eligibility criteria, demographics, medical history, concomitant medications, vital signs, height and weight measurements, serum pregnancy test (for female subjects of childbearing potential only), clinical laboratory tests, MMSE, audiometry, otoscopy, DIN test, and pre-dose plasma PK and immunogenicity samples. Subjects must exhibit a speech-in-noise hearing deficit in at least one ear (study ear) as indicated by the DIN test at Screening.
In addition, for Cohorts 1-4, tympanometry, DPOAE, and auditory-evoked potentials are conducted at any time during the Screening period but are not needed to determine eligibility. Likewise, the WIN, AEMT, MEMR, and SSQ-12 assessments are conducted during Screening but are not needed to determine eligibility (note: these four tests are administered twice during the period from Screening to Baseline/investigational product injection, with at least a 6-day interval between the test administrations).
For Cohorts 5-7, the following assessments, as listed in the Time and Events Schedule Error! Reference source not found, will be performed during the Screening period: informed consent, review and confirm eligibility criteria, demographics, medical history, concomitant medications, vital signs, height and weight measurements, serum pregnancy test (for female subjects of childbearing potential only), clinical laboratory tests, MMSE, audiometry, otoscopy, DIN test, and pre-dose plasma immunogenicity samples. Subjects must exhibit a speech-in-noise hearing deficit in at least one ear (study ear) as indicated by the DIN test at Screening.
In addition, for Cohorts 5-7, tympanometry is conducted at any time during the Screening period but is not needed to determine eligibility. Likewise, the WIN and AEMT are conducted during Screening but are not needed to determine eligibility (note: these 3 tests are administered twice during the period from Screening to Baseline/investigational product injection, with at least a 6-day interval between the test administrations).

### 7.1.2. Baseline/Investigational Product Administration: Day 1

Once a subject has signed consent, completed the Screening assessments, and met all study eligibility criteria, the subject will return to the clinical site for Baseline procedures and Investigational Product administration.
For Cohorts 1-4, pre-dose Baseline assessments including the DIN, WIN, AEMT, MEMR, and SSQ-12 test will be performed at the Baseline visit or up to 7 days prior to the Baseline visit.
For Cohorts 5-7, pre-dose Baseline assessments including the DIN, WIN, and AEMT will be performed at the Baseline visit or up to 7 days prior to the Baseline visit.
Results from the concomitant medications review and urine pregnancy test (for female subjects of childbearing potential) must be available and reviewed by the Investigator to confirm the subject's eligibility before randomization. In addition, the following assessments are to be performed on all subjects prior to dosing at the Baseline visit: medical history (changes since the Screening visit), vital signs, otoscopy, and C-SSRS (“Baseline” version).
If the subject is no longer eligible, the subject will not be randomized and should be recorded as a screen failure. Information related to specific inclusion/exclusion criteria will be documented. Once eligibility status is confirmed and the subject is randomized, the investigational product is administered and the remaining Day 1 assessments (e.g., PK sampling at 1 hour [±15 minutes] post-dose for Cohorts 1-4 and Cohorts 6 and 7, AE monitoring) are completed.
All assessments as listed in the Time and Events Schedule for Cohorts 1-4 (Error! Reference source not found.) or Cohorts 5-7 (Error! Reference source not found.) are to be performed at this visit.
7.1.3. Day 8 (±2 days): Follow Up
For all cohorts, the primary purpose of the Day 8 visit is to capture safety and PK data. The following assessments are to be performed: concomitant medications, AE monitoring, vital signs, clinical laboratory tests, audiometry, otoscopy, tympanometry, and C-SSRS. A final plasma PK sample (Cohorts 1-4 and Cohorts 6-7) and a plasma sample for immunogenicity testing will be obtained at this visit (at any time during the visit).
All assessments as listed in the Time and Events Schedule for Cohorts 1-4 (Error! Reference source not found.) or Cohorts 5-7 (Error! Reference source not found.) are to be performed at this visit.
7.1.4. Day 15 (±2 days): Follow Up
   For Cohorts 1-4 only, the primary purpose of the Day 15 visit is to capture safety and exploratory efficacy data. Safety assessments include concomitant medications, AE monitoring, vital signs, audiometry, otoscopy, tympanometry, and C-SSRS. Exploratory efficacy assessments include DPOAE, DIN, WIN, AEMT, auditory-evoked potentials, MEMR, SSQ-12, and PGIC.
   All Day 15 safety and exploratory efficacy assessments as listed in the Time and Events Schedule (Error! Reference source not found.) are to be performed.
   No Day 15 visit is conducted for Cohorts 5-7 subjects.
   7.1.5. Day 29 (±2 days): Follow Up
   For all cohorts, the primary purpose of the Day 29 visit is to capture safety and exploratory efficacy data. Safety assessments include concomitant medications, AE monitoring, vital signs, clinical laboratory tests, audiometry, otoscopy, tympanometry, and C-SSRS.
   For Cohorts 1-4, exploratory efficacy assessments include DPOAE, DIN, WIN, AEMT, auditory-evoked potentials, MEMR, SSQ-12, and PGIC.
   For Cohorts 5-7 subjects, exploratory efficacy assessments include the DIN, WIN, AEMT, and PGIC.
   For all cohorts, a plasma sample will be obtained for immunogenicity testing at any time during this visit.
   All Day 29 safety and exploratory efficacy assessments as listed in the Time and Events Schedule for Cohorts 1-4 (Error! Reference source not found.) or Cohorts 5-7 (Error! Reference source not found.) are to be performed.

### 7.1.6. Day 57 (±3 Days): Follow Up

For all cohorts, the primary purpose of the Day 57 visit is to capture safety and exploratory efficacy data. Safety assessments include concomitant medications, AE monitoring, vital signs, audiometry, otoscopy, tympanometry, and C-SSRS.
For Cohorts 1-4, exploratory efficacy assessments include DPOAE, DIN, WIN, AMET, auditory-evoked potentials, MEMR, SSQ-12, and PGIC.
For Cohorts 5-7 subjects, exploratory efficacy assessments include the DIN, WIN, AEMT, and PGIC.
All Day 57 safety and exploratory efficacy assessments as listed in the Time and Events Schedule for Cohort 1-4 (Error! Reference source not found.) or Cohorts 5-7 (Error! Reference source not found.) are to be performed.

### 7.1.7. Day 85 (±4 Days): End-of-Study/Early Termination

For all cohorts, the primary purpose of Day 85 visit is to capture safety and exploratory efficacy data. Safety assessments include concomitant medications, AE monitoring, vital signs, weight, urine pregnancy test (for female subjects of childbearing potential), clinical laboratory tests, tympanometry, audiometry, otoscopy, and C-SSRS.
For Cohorts 1-4, exploratory efficacy assessments include DPOAE, DIN, WIN, AEMT, MEMR, SSQ-12, and PGIC.
For Cohorts 5-7, exploratory efficacy assessments include DIN, WIN, AEMT, and PGIC.
For all cohorts, a final plasma sample will be obtained for immunogenicity testing at any time during this visit.
All Day 85 safety and efficacy assessments as listed in the Time and Events Schedule for Cohorts 1-4 (Error! Reference source not found.) or Cohorts 5-7 (Error! Reference source not found.) are to be performed. Ha subject terminates from the study prior to Day 85 (Early-Termination), every attempt should be made to perform all assessments as listed for Day 85.

### 7.1.8. Unscheduled Visit

Unscheduled Visits may occur in the event of safety-related issues. Appropriate safety assessments (e.g., otoscopy, vital signs, clinical laboratory tests) may be conducted at the Investigator's discretion at Unscheduled Visits.

### 7.2. Medical History and Demographics

The medical history will be obtained from medical records and/or via subject interview at the Screening visit, and includes general medical history, medication history, reproductive history, and hearing and noise exposure history.
Demographic information will also be obtained at the Screening visit and will include age; sex; race/ethnicity; and highest level of education completed.

### 7.3. Mini-Mental State Examination

The Mini-Mental State Examination (MMSE) is a widely used cognitive screening test with good test-retest and inter-rater reliability (Folstein 1975). The MMSE assesses orientation, memory, attention, language, and motor skills.
The MMSE is administered at Screening.

### 7.4. Efficacy Evaluations

Exploratory efficacy assessments include:
Electrophysiological Measures (Cohorts 1-4)
   Auditory-evoked potentials
   Middle Ear Muscle Reflex (MEMR)
Speech-in-Noise Tests (All Cohorts)
   Digits-in-Noise (DIN) Test
   Words-in-Noise (WIN) Test
   American English Matrix (AEMT) Test
Patient Reported Hearing Ability
   Speech, Spatial and Qualities of Hearing Scale 12 (SSQ-12) Test (Cohorts 1-4)
   Patient Global Impression of Change (PGIC) (All Cohorts)
Hearing Function Tests
   DPOAE (Cohorts 1-4)
      The Study Procedures Manual contains detailed instructions for each assessment and suggested order of assessment.

### 7.4.1. Digits-in-Noise Test (DIN)

The Digits-in-Noise test (DIN) is a hearing-in-noise test in which 3 spoken numbers (referred to as digit triplets) are presented at varying sound intensities through earphones, while a continuous noise at a fixed level is presented synchronously (Smits et al 2013). A speech reception threshold (SRT) is determined from the signal-to-noise ratio (SNR) for 50% correct for whole triplets.
Subjects in Cohorts 1-4 will be administered the DIN test in each ear at Screening and in study ear only at Day 1 (pre-dose), and Days 15, 29, 57, and 85. Subjects in Cohorts 5-7 will be administered the DIN in each ear at Screening and in study ear only at Day 1 (pre-dose), and Days 29, 57, and 85.

### 7.4.2. Words-in-Noise Test (WIN)

The Words-in-Noise test (WIN) is a word recognition test that uses multi-talker babble as the background noise as spoken words are presented (Wilson et al 2007). Both the multi-talker babble and spoken words are introduced through earphones. Five unique words, from the Northwestern University Auditory Test No. 6, are spoken by a female speaker. The presentation level of the multi-talker babble is fixed, and the words are presented at varying signal-to-babble ratios. The test is scored as the signal-to-noise ratio at which word recognition performance is 50% as determined by the Spearman-Karber equation.
Subjects in all Cohorts 1-4 will be administered the WIN test in each ear at Screening and in study ear only at Day 1 (pre-dose) and Days 15, 29, 57, and 85. Subjects in Cohorts 5-7 will be administered the WIN in each ear at Screening and in study ear only at Day 1 (pre-dose), and Days 29, 57, and 85.

### 7.4.3. American English Matrix Test (AEMT)

The American English Matrix Test (AEMT) is an adaptive speech-in-noise test using steady-state noise that is held constant as the sentence sound level is varied (Kollmeier et al 2015). The test uses grammatically correct but semantically unpredictable sentences with a fixed syntactic structure (name-verb-numeral-adjective-object, e.g., “Rachel wants four pretty chairs”). The sentences are pre-constructed by making random combinations of 5 words, one from each category of an inventory of 50 words. The low semantic predictability minimizes learning effects.
Subjects in Cohorts 1-4 will be administered th'e AEMT in each ear at Screening and study ear only at Day 1 (pre-dose) and Days 15, 29, 57, and 85. Subjects in Cohorts 5-7 will be administered the AEMT in each ear at Screening and in study ear only at Day 1 (pre-dose), and Days 29, 57, and 85.

### 7.4.4. Auditory-Evoked Potentials

Auditory-evoked potentials are electrophysiological tests of auditory brainstem and subcortical function in response to auditory stimuli. An auditory-evoked potential is elicited by a brief click or tone transmitted from an acoustic transducer from an insert electrode or earphone. The elicited waveform response is measured by surface electrodes typically placed on the scalp/forehead and earlobes. The elicited waveforms reflect neural activity at various points along the auditory pathway (e.g., for the auditory brainstem response [ABR] Wave I represents afferent neural activity of the distal portion of the auditory nerve, while Wave V reflects downstream neural activity in the vicinity of the inferior colliculus) (Boston and Moller 1985). Auditory-evoked potentials have been investigated as measures of cochlear synaptopathy and changes in individual wave amplitudes, latencies, and envelope-following responses have been associated, in some studies, in subjects with high noise exposure and high frequency hearing loss (Barbee et al., 2018; Bharadwaj et al., 2019).
Cohorts 1-4 subjects will undergo auditory-evoked potential testing in study ear only at Screening and Days 15, 29, and 57. Cohorts 5-7 subjects will not undergo auditory-evoked potential testing.

### 7.4.5. Middle Ear Muscle Reflex (MEMR)

The middle ear muscle reflex (MEMR) is an acoustic reflex that is used clinically to assess middle ear function. The reflex isan involuntary muscle contraction of the stapedius muscle in response to a high-intensity sound stimulus that reduces transmission through the middle ear (Mukerji et al., 2010). The MEMR and the acoustic threshold for activating the MEMR is sensitive to the presence of cochlear synaptopathy in animal studies (Valero et al., 2016) and the MEMR amplitude strength was reduced in patients with tinnitus and normal hearing thresholds, where cochlear synaptopathy is suspected (Wojtczak et al., 2017).
Cohorts 1-4 subjects will undergo MEMR testing in each ear at Screening and in study ear only at Day 1 (pre-dose) and Days 15, 29, 57, and 85. Cohorts 5-7 subjects will not undergo MEMR testing.

### 7.4.6. Distortion Product Otoacoustic Emissions (DPOAE)

Distortion Product Otoacoustic Emission (DPOAE) assesses outer hair cell integrity and cochlear function (Abdala and Visser-Dumont, 2001).
For Cohorts 1-4 subjects, DPOAE testing will be performed in each ear at Screening and in study ear only at Days 15, 29, 57, and 85. Cohorts 5-7 subjects will not undergo DPOAE testing.

### 7.4.7. Short Form of the Speech, Spatial and Qualities of Hearing Scale (SSQ-12) Test

The short form of the Speech, Spatial and Qualities of Hearing Scale (SSQ-12) is a 12-item self-report questionnaire that assesses speech hearing, spatial hearing reflecting directional and distance judgments, qualities of hearing, and listening effort (Noble et al., 2013).
Cohorts 1-4 subjects will complete the SSQ-12 at Screening, Day 1 (pre-dose) and Days 15, 29, 57, and 85. Cohorts 5-7 subjects will not complete the SSQ-12.

### 7.4.8. Patient Global Impression of Change (PGIC)

The PGIC is a patient-reported outcome that evaluates the change in overall “global” hearing status as perceived by the subject. The subject is asked: “Since the beginning of the clinical study, how would you rate your ability to hear in noise?”. The beginning of the clinical study in this context is the time prior to investigational product administration.
Cohort 1-4s subjects will complete the PGIC at Days 15, 29, 57, and 85. Cohorts 5-7 subjects will complete the PGIC at Days 29, 57, and 85.

### 7.5. Blood Sampling for Plasma PK Evaluations

For Cohorts 1-4 and Cohorts 6 and 7, blood samples for plasma BDNF will be collected from all subjects.
A pre-dose sample will be collected at Screening and post-dose on Day 1 at 1 hour (±15 minutes) after intratympanic administration. On Day 8, a final PK sample will be obtained at any time during the study visit.
Collection, storage, and shipping of plasma PK samples for BDNF will be performed as outlined in the Laboratory Manual. E′very attempt should be made to collect samples at the protocol-specified times.
Plasma samples will be evaluated for BDNF using validated analytical procedures by a bioanalytical laboratory.

### 7.6. Blood Sampling for Immunogenicity Testing

For all cohorts, blood samples for plasma immunogenicity testing will be collected from all subjects. A pre-dose sample will be collected at Screening and post-dose samples will be collected at any time during visits on Days 8, 29, and 85.
Collection, storage, and shipping of blood samples for immunogenicity testing will be performed as outlined in the Laboratory Manual.
Plasma samples will be evaluated for anti-BDNF antibodies using validated analytical procedures by a bioanalytical laboratory. In addition, plasma samples may be evaluated for BDNF in Cohort 5, if warranted, using validated analytical procedures.

### 7.7. Safety Evaluations

Safety assessments include:
Vital Signs and Weight Measurements
Clinical Laboratory Tests (Hematology, Serum Chemistry, and Urinalysis)
Tympanometry
Audiometry
Otoscopy
C-SSRS Assessment
Concomitant Medications (see Section 6)
Adverse events (see Section 8)

### 7.7.1. Vital Signs and Height/Weight Measurements

Vital signs measurements (including systolic and diastolic blood pressure, pulse rate, body temperature, and respiratory rate) will be collected at Screening, Day 1 (pre-dose), and Days 8, 15, 29, 57, and 85. No Day 15 visit is conducted for Cohorts 5-7 subjects.
Vital signs will be measured after subjects have been seated for 5 minutes and while subjects are in a sitting position.
Height and body weight (both without shoes) are measured at Screening. Weight will also be measured at Day 85 visit.

### 7.7.2. Clinical Laboratory Tests

All clinical laboratory tests (except for urine pregnancy) will be processed by a Central Laboratory.
Blood and urine samples for hematology, serum chemistry, urinalysis, and pregnancy tests will be prepared using standard procedures.
For all cohorts, clinical laboratory testing will be completed at Screening, Day 8, Day 29, and Day 85. In addition, female subjects of childbearing potential will have serum pregnancy test at Screening and a urine pregnancy test at Day 1 (pre-dose) and Day 85. The urine pregnancy tests will be performed locally at the site, so results are available that day.
The blood and urine samples will be used for the following tests:
Hematology: hemoglobin, hematocrit, red blood cell count, white blood cell count with differential, mean corpuscular volume, mean corpuscular hemoglobin, mean corpuscular hemoglobin concentration, and platelet count.
Serum chemistry: albumin, alkaline phosphatase, alanine aminotransferase, aspartate aminotransferase, gamma glutamyl-transpeptidase, bicarbonate, blood urea nitrogen, calcium, chloride, creatinine, glucose, lactate dehydrogenase, phosphorus, potassium, sodium, total bilirubin, and total protein.
Urinalysis: appearance, color, pH, specific gravity, protein, glucose, ketones, nitrite, leukocyte esterase, blood, urobilinogen, and reflex testing for microscopic sediment examination if necessary.

### 7.7.3. Tympanometry

Tympanometry assessments will be used to assess the mobility and compliance of the tympanic membrane, pressure and volume in the outer ear canal, and function of the tympanic membrane, ossicles and eustachian tube.
For Cohorts 1-4, tympanograms will be completed in each ear at Screening and in study ear only at Days 8, 15, 29, 57, and 85, unless the examiner determines that there is a contraindication to performing the procedure.
For Cohorts 5-7, tympanograms will be completed in each ear at Screening and in study ear only at Days 8, 29, 57, and 85, unless the examiner determines that there is a contraindication to performing the procedure.

### 7.7.4. Audiometry

Audiometric assessments must be conducted in accordance with American-Speech-Language-Hearing Association Guidelines (ASHA, 2005). Equipment calibration must be current and documented. The audiometric assessments must be conducted by a licensed or certified audiologist or a qualified assistant with appropriate training under the direct supervision of a licensed or certified audiologist.
Audiograms are conducted for air conduction at 250, 500, 1000, 2000, 3000, 4000, 6000, and 8000 Hz for air conduction and at 1000, 2000, and 4000 Hz for bone conduction. Additional high frequency testing is also required (see Study Procedure Manual). Both air and bone conduction thresholds will be assessed.
For Cohorts 1-4, audiometry will be used to assess hearing function in each ear at Screening and in study ear only at Days 8, 15, 29, 57, and 85.
For Cohorts 5-7, audiometry will be used to assess hearing function in each ear at Screening and in study ear only at Days 8, 29, 57, and 85.

### 7.7.5. Otoscopy

Otoscopic exams will be used to assess the auditory canal, the appearance of the tympanic membrane, and the healing of the intratympanic injection site. Presence and size of tympanic membrane perforations will be recorded. Perforations of the tympanic membrane will be captured as AEs if the perforation does not resolve by the end of the study or increases in size.
For Cohorts 1-4, otoscopic examinations will be performed by the physician or qualified healthcare professional in each ear at Screening and in study ear only at Day 1 (pre-dose) and Days 8, 15, 29, 57, and 85.
For Cohorts 5-7, otoscopic examinations will be performed by the physician or qualified healthcare professional in each ear at Screening and in study ear only at Day 1 (pre-dose) and Days 8, 29, 57, and 85.

### 7.7.6. C-SSRS Assessment

The C-SSRS is a scale that captures the occurrence, severity, and frequency of suicide-related thoughts and behaviors during the assessment period (Posner 2011). The U.S. Food and Drug Administration requires that a prospective assessment for suicidal ideation and behavior be included in clinical studies involving all drugs and biological products for neurological indications. This is true whether or not a particular product is known or suspected to be associated with treatment-emergent suicidal ideation and behavior. The C-SSRS fulfills this requirement. The scale includes suggested questions to solicit the type of information needed to determine if a suicide-related thought or behavior occurred. The C-SSRS must be administered by appropriately trained and certified personnel.
For Cohorts 1-4, the C-SSRS assessment will be administered at Day 1 (pre-dose), and Days 8, 15, 29, 57, and 85. For Cohorts 5-7, the C-SSRS assessment will be administered at Day 1 (pre-dose), and Days 8, 29, 57, and 85. The “Baseline” version will be used at Day 1. For all other days, the “Since Last Visit” version will be used.
Any subject deemed by the Investigator to be at significant risk of suicidal behavior should be excluded. Any subject with a new suicidal ideation or suicidal behavior on the “Since Last Visit” version should be evaluated by a clinician/mental health professional skilled in the evaluation of suicidal ideation and behavior (e.g., psychiatrist, licensed clinical psychologist) who will determine if it is safe for the subject to participate/continue in the study.

### 8. Adverse Event Reporting

Timely, accurate, and complete reporting and analysis of safety information from clinical trials will be conducted in accordance with Good Clinical Practice.
All AEs and serious adverse events (SAEs) that are reported or observed during or after dosing with the investigational product will be recorded on the AE page of the eCRF for all enrolled subjects. Medical conditions existing before administration of investigational product will be recorded as part of medical history. Information to be collected includes description of event, date of onset, Investigator-specified assessment of the severity and relationship to investigational product, relationship to the intratympanic injection, date of resolution of the event, seriousness, any required treatment or evaluations, and outcome. Adverse events resulting from concurrent illnesses, reactions to concurrent illnesses, reactions to concurrent medications, or progression of disease states must also be reported. Perforations of the tympanic membrane will be captured as AEs if the perforation does not resolve by the end of the study or increases in size.
If the existing medical condition worsens at any time after the injection, it should be recorded as an AE.

### 8.1. Adverse Event Classification Definitions

### Adverse Event:

An AE is any unfavorable and unintended diagnosis, symptom, sign, syndrome, or disease which occurs during the study, having been absent at baseline, or, if present at baseline, appears to worsen.
This includes any occurrence that is new in onset or aggravated in severity or frequency from the baseline condition, including abnormal results of diagnostic procedures and/or laboratory test abnormalities, which are considered AEs if they:
result in discontinuation from the study,
require treatment or any other therapeutic intervention,
require further diagnostic evaluation (excluding a repetition of the same procedure to confirm the abnormality), or
are associated with clinical signs or symptoms judged by the Investigator to have a significant clinical impact

### Serious Adverse Event (SAE):

An SAE is defined as any untoward medical occurrence that:
results in death,
is life-threatening (Note: the term “life-threatening” refers to an event in which the subject was at risk of death at the time of the event rather than to an event which hypothetically might have caused death if it were more severe.),
requires in-patient hospitalization or prolongation of existing hospitalization,
results in persistent or significant disability/incapacity, or
is a congenital anomaly/birth defect.
   Medical and scientific judgment should be exercised in deciding whether expedited reporting is appropriate in other situations, such as important medical events that may not be immediately life-threatening or result in death or hospitalization but may jeopardize the subject or may require intervention to prevent one of the other outcomes listed in the above definition. These events should be considered serious.
9. Subject Completion
9.1. Completion

### PCT/US2021/010053

Study subject participation is complete after Day 85 (Visit 7). Subjects who withdraw their consent to be followed or are lost-to-follow-up before completion of Day 85 will not be considered to have completed the study.
9.2. Withdrawal
All subjects have the right to withdraw from study evaluations at any time, for any reason, without prejudice; nonetheless, Investigators should attempt to encourage subjects to complete the protocol so that continued observation and follow-up measurements may be obtained.
   Subjects must be withdrawn from the study for any of the following:
withdrawal of consent, or
the subject is unwilling or unable to comply with the protocol.
   Other reasons for withdrawal of subjects from the study might include:
at the discretion of the Investigator for medical reasons,
at the discretion of the Investigator or Sponsor for noncompliance,
significant protocol deviation, or
decision by the Investigator or Sponsor
   At any point, the Investigator may discontinue the subject's study participation at the discretion of the Investigator or at the request of the subject, and ensure the subject receives appropriate medical care; the Investigator may also consult the medical monitor to discuss out-of-range test results.

### 9.2.1. Handling of Withdrawals

Subjects will be free to withdraw from the study, including discontinuing investigational product administration, and further follow-up of the study at any time.
Should a request for early withdrawal from the study with no further follow-up be made, the subject should be encouraged to return to the study site for a last follow up visit and undergo all End-of-Study/Early-Termination assessments.
When a subject withdraws from the study prior to completing the End-of-Study Visit, the reason for withdrawal is to be documented on the eCRFs and in the source document.

### 9.2.2. Replacements

In order to meet the target for 8 evaluable subjects for each escalating cohort for Cohorts 1-4 and to meet the target of 12 evaluable subjects for each escalating cohort for Cohorts 6 and 7, subjects who discontinue participation in the study for non-safety related reasons may be replaced at the discretion of the Sponsor.
In Cohort 5, subjects who discontinue participation prior to week 8 for non-safety related reasons, may be replaced at the discretion of the Sponsor.

### 10. Statistical Methods

### 10.1. Sample Size

The sample size for each dose level cohort was selected based on clinical judgment and prior experience to ensure that the safety and tolerability will be adequately assessed while minimizing subject exposure. Some cohorts may be expanded to further the experience with a particular dose level, using the 3:1 (COMPOSITION A:placebo) allocation ratio (Cohort 4) or the 2:1 (COMPOSITION A:placebo) allocation ratio (Cohorts 5-7).

### 10.2. Analysis Sets

The Safety analysis set includes all subjects randomized and dosed. Subjects will be included in the group based on the treatment that was received. The Safety analysis set will be used for all summaries of safety.
The Per Protocol analysis set includes all subjects that are randomized, dosed, had no major protocol deviations, and at least one post-dose efficacy visit completed. Subjects will be included in the group based on the treatment that was received. All exploratory efficacy analyses will be carried out in the Per Protocol analysis set.
Additional details on these and other analysis sets are provided in the statistical analysis plan.

### 10.3. Subject Demographics, Baseline Disease Status, and Disposition

Descriptive statistics for subject demographics, baseline disease status, and subject disposition will be provided by cohort.
10.4. Study Endpoints
10.4.1. Safety Endpoints
The safety endpoints for all cohorts are:
Assessment of treatment-emergent adverse events (TEAEs).
Clinically significant adverse change from Baseline in audiometry assessments, tympanometry, and otoscopic examinations.
Clinically significant adverse change from Baseline in clinical laboratory measurements (hematology, serum chemistry, and urinalysis) and vital signs measurements (systolic and diastolic blood pressure, pulse rate, body temperature, and respiratory rate).
Assessment of suicidality via the C-SSRS.

### 10.4.2. Exploratory Efficacy Endpoints

Exploratory efficacy endpoints are:

### Cohorts 1-4

The change from baseline in auditory-evoked potentials and MEMR as electrophysiological measures of cochlear synaptopathy.
The change from baseline in the signal-noise ratios (SNRs) for the DIN, WIN, and AEMT as assessments of speech-in-noise hearing.
The PGIC score and the change from baseline in the SSQ-12 as patient-reported outcomes of hearing ability.
The change from baseline in DPOAE and audiometry as measures of hearing function.

### Cohorts 5-7

The change from baseline in the signal-noise ratios (SNRs) for the DIN, WIN, and AEMT assessments of speech-in-noise hearing.
The PGIC score as a self-reported outcome of hearing ability.
   Changes from baseline at the individual subject level, as well as group means, will be evaluated.

### 10.4.3. Plasma PK and Immunogenicity Endpoints

For Cohorts 1-4 and Cohorts 6-7, the plasma PK endpoint is:
Plasma concentrations of BDNF. Note, for Cohort 5, plasma BDNF may be evaluated if warranted.
   For all cohorts, the immunogenicity endpoint is:
Anti-BDNF antibodies.

### 10.4.4. Analytic Methods for Efficacy

Descriptive statistical tabulations will be presented for all exploratory efficacy subject data.

### 10.5. Safety Evaluations

Safety endpoints to be examined include:
Adverse events
Vital Signs and Weight Measurements
Clinical Laboratory Tests
Tympanometry
Audiometry
Otoscopy
C-SSRS Assessment
Concomitant Medications
   Descriptive statistical tabulations will be presented for all subjects included in the Safety analysis set.

### 10.5.1. Adverse Events

The current version of Medical Dictionary for Regulatory Activities (MedDRA), as indicated in the Data Management Plan, will be used to code all AEs.
The primary analysis of AEs will consider only treatment-emergent adverse events (TEAEs), events occurring for the first time, or worsening during or after the first dose of investigational product. Subject incidence of TEAEs and SAEs will be tabulated by Preferred Terms (PTs) and System Organ Class (SOC). Severity and relationship to investigational product will also be presented. For summary tables, a subject who experiences the same coded event more than once is counted only one time for that coded event at the highest severity level. AEs will be presented by descending order of frequency in MedDRA SOC and PT.
Listings of all SAEs, AEs leading to study withdrawal, and deaths on-study will also be included. Duration and outcome of each AE will be reported in subject listings.

### 10.5.2. Vital Signs and Laboratory Parameters

The analysis of vital signs and laboratory parameters will include descriptive statistics for the change from Baseline (baseline assessment at Screening for laboratory parameters) to the endpoint visit and change from Baseline for each visit (vital signs only). Where appropriate, analyses will also include shifts from Baseline to the endpoint visit. For laboratory values, the normal ranges will be used to determine the classifications. Values below the normal range will be classified as low, values above the normal range will be classified as high, and values within the normal range will be classified as normal.

### 10.5.3. Otoscopic Examinations

Observations recorded during the conduct of otoscopic exams will be descriptive in nature. The number and percent of subjects presenting with each Otoscopic classification will be provided by treatment group and study visit. Where relevant, the number and proportion of subjects with changes in their otoscopic classification from Baseline to the endpoint visit will also be provided for each treatment group. All otoscopic assessments will be tabulated separately for the study and non-study ear.

### 10.5.4. Audiometry Assessments

Descriptive summary statistics for audiometric assessments of air and bone conduction thresholds at each frequency will be provided by treatment group and study visit. All audiometry assessments will be tabulated separately for the study and non-study ear.

### 10.5.5. Tympanometry

Shift tables representing the proportion of subjects with changes in their tympanogram from Baseline to each post-Baseline study visit will be calculated for each treatment group. Tympanogram changes will include the type of tympanogram (A, B-small volume and/or normal, B-large volume, or C). All tympanometry assessments will be tabulated separately for the study and non-study ear.

### 10.5.6. Columbia-Suicide Severity Rating Scale (C-SSRS)

All suicidal ideation and behavior variables will be tabulated overall and by treatment group for each study visit. All C-SSRS data will be included in data listings.

### 10.6. Handling of Missing Data, Subject Withdrawals, and Treatment Failures

Every reasonable effort will be made to encourage compliance with study measurement methods and study procedures to minimize missing data.
Every reasonable effort will be made to follow subjects who withdraw from the study for safety evaluation until the date they would have completed participation.

### 10.7. Interim Analyses

There are no formal interim analyses planned. Blinded reviews of safety data will be conducted as described in Section 2.1; however, the review of such data is not intended to impact the study conduct unless there are safety concerns. As such, it is expected that the study will continue to its scheduled completion barring any unexpected safety issues.

### 11. Investigational Product Information

### 11.1. Physical Description of Investigational Product

The investigational product administered to subjects will be COMPOSITION A and placebo. COMPOSITION A is prepared by diluting the BDNF concentrate solution with the diluents provided.
The BDNF concentrate (9 mg/mL) in phosphate buffered saline is provided sterile in a vial inside a kit that is stored at −20° C. until use. The Placebo, 16% P407 poloxamer, is provided sterile in a vial inside a kit that is stored at 2-8° C. until use. Diluents containing P407 are provided in vials and stored at 2-8° C. until use. The diluents are provided separately and used, by unblinded qualified research staff, to dilute the BDNF concentrate to the target concentration for the COMPOSITION A dose level (see Pharmacy Manual for investigational product preparation procedures).

### 11.2. Directions for Use

COMPOSITION A and placebo syringes will be prepared in a clean, secure location at room temperature by unblinded qualified research staff. Refer to the Pharmacy Manual for detailed investigational product preparation instructions.

### 11.3. Packaging and Labeling

### 11.3.1. Packaging

BDNF concentrate and Placebo kits will be labeled with information that will meet the applicable regulatory requirements.

### 11.3.2. Labels and Labeling Instructions

A label will be affixed to each kit box indicating kit number and storage instructions. A label will be affixed to the BDNF concentrate, Placebo, Diluent A, Diluent B, and Diluent C vials indicating contents and storage instructions.

### 11.4. Management of Clinical Supplies

The clinical supplies will be managed by IWR randomization system and an unblinded investigational product manager. Investigational product inventory and re-supply will be overseen by an unblinded IP manager and these requests will be manually submitted by them to the Almac depot. Upon shipment and receipt of the cliniCal study material, the unblinded site personnel will acknowledge the shipment and identify any damaged, missing, or unusable kits so they will not be dispensed.

### 11.4.1. Storage of Kits

Kits containing BDNF concentrate will be stored at −20° C. (−25° C. to −10° C.). Placebo kits and Diluent vials will be stored at 2-8° C. All temperature excursions of the investigational product must be documented in the investigational product accountability log. The pharmacy manual should be referenced if the investigational product falls outside of these conditions. The manual contains guidance on who to contact if an excursion occurs and outlines the process for confirming whether the investigational product is acceptable for use in treating subjects. If the contact determines that the investigational product is not considered acceptable for use, the individual preparing the investigational product should immediately quarantine the product and report the kit(s) as unacceptable for dispensing to the IWR to remove it from inventory.

### Preliminary Results of Clinical Trial (Cohorts 1-4)

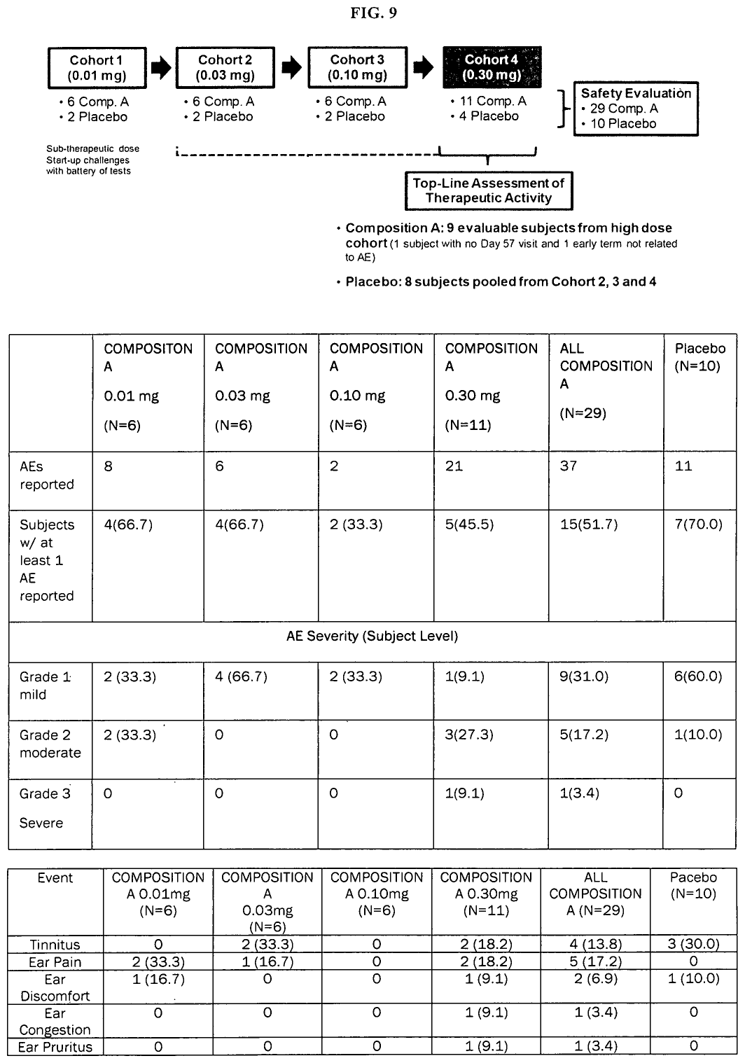

For Cohorts 1-4, FIG. 9 illustrates subject disposition (top), overall adverse events (middle), and ear related adverse events (bottom). COMPOSITION A was well tolerated across all dose cohorts with 52% of COMPOSITION A subjects versus 70% of placebo subjects reporting >1 adverse event. There was no apparent impact of dose on AE incidence across the cohorts. Most of the COMPOSITION A reported AEs were mild 28/37 (76%) or moderate 8/37 (22%) in severity. No Serious Adverse Events (SAEs) were reported. One AE of Diarrhea (due to COVID-19) was reported as Grade 3 and was not related to study drug. For ear related adverse events, AEs of the Ear and Labyrinth Disorders System Organ Class were the most frequently occurring AEs. The occurrence of ear related adverse events between COMPOSITION A and placebo treated subjects were comparable. One of the 4 tinnitus AEs for the COMPOSITION A treated subjects was deemed not related to study treatment and occurred in the non-injected ear; the tinnitus for this subject resolved within a day.

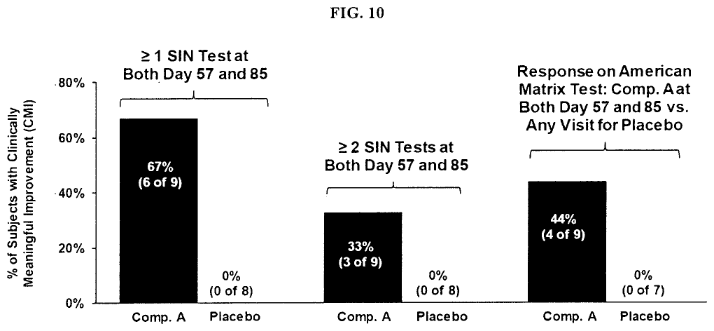

The overall clinical efficacy is summarized in FIG. 10, which shows that 6 of 9 (67%) subjects treated with the highest dose of COMPOSITION A (0.30 mg) showed a clinically meaningful improvement on at least one of the three speech-in-noise tests at both Day 57 and 85 versus 0 of 8 (0%) for placebo. In addition, 3 out of 9 (33%) subjects treated with the highest dose of COMPOSITION A (0.30 mg) showed a clinically meaningful improvement on at least two of the three speech-in-noise tests at both Day 57 and 85 versus 0 of 8 (0%) for placebo. Performance on the sentence-based AEMT favored COMPOSITION A, with 4 of 9 (44%) COMPOSITION A subjects experiencing a clinically meaningful improvement at both Day 57 and 85 compared to 0 of 7 (0%) placebo subjects at any single timepoint.

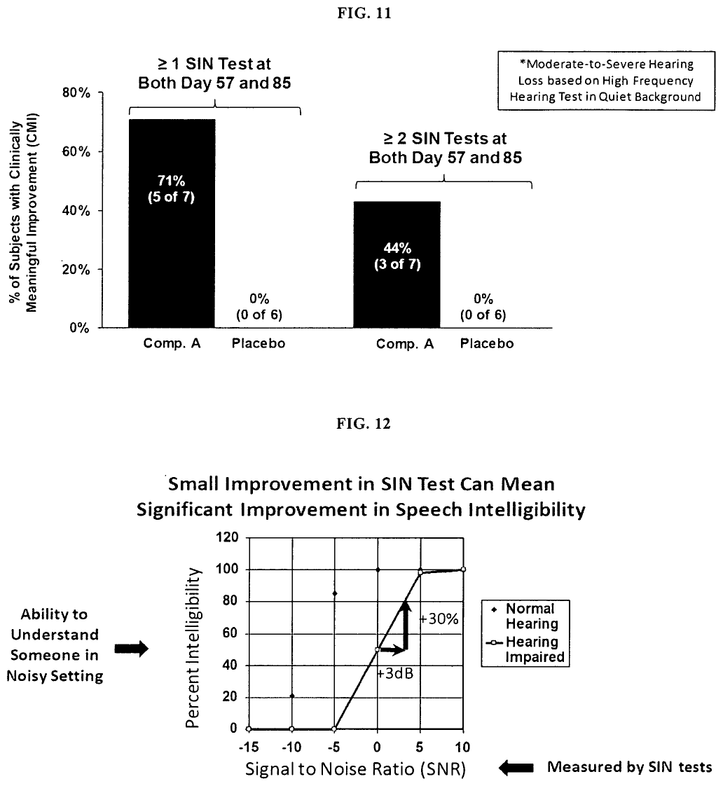

The clinical efficacy for a subset of the patients with moderate-to-severe hearing loss is summarized in FIG. 11, which shows that 5 of 7 (71%) subjects treated with the highest dose of COMPOSITION A (0.30 mg) showed a clinically meaningful improvement on at least one of the three speech-in-noise tests at both Day 57 and 85 versus 0 of 6 (0%) for placebo. In addition, 3 out of 7 (44%) subjects treated with the highest dose of COMPOSITION A (0.30 mg) showed a clinically meaningful improvement on at least two of the three speech-in-noise tests at both Day 57 and 85 versus 0 of 6 (0%) for placebo.

A clinically meaningful improvement was defined as a minimum change of −3 dB SNR (DIN) or −2 dB SNR (WIN and AEMT). As illustrated in FIG. 12, a small improvement in SIN tests can correspond to a significant improvement in speech intelligibility.

While preferred embodiments of the present disclosure have been shown and described herein, such embodiments are provided by way of example only. It is not intended that the invention be limited by the specific examples provided within the specification. Various alternatives to the embodiments described herein are optionally employed. Furthermore, it shall be understood that all aspects of the invention are not limited to the specific depictions, configurations or relative proportions set forth herein which depend upon a variety of conditions and variables. It is intended that the following claims define the scope of the disclosure and that methods and structures within the scope of these claims and their equivalents be covered thereby.

What is claimed is:

1. A method of treating hearing loss or hearing impairement in a human subject, comprising intratympanically administering an otic formulation to the human subject, wherein the otic composition comprises from about 0.005 mg to about 1.90 mg of brain-derived neurotrophic factor (BDNF) and an auris-acceptable vehicle, wherein the otic formulation is formulated to provide sustained release of BDNF into the inner ear.

2. The method of claim 1, wherein the BDNF is a recombinant BDNF.

3. The method of claim 1, wherein the composition comprises from 0.005 mg to 0.015 mg BDNF.

4. The method of claim 3, wherein the composition comprises about 0.01 mg BDNF.

5. The method of claim 1, wherein the composition comprises from 0.015 mg to 0.05 mg BDNF.

6. The method of claim 5, wherein the composition comprises about 0.03 mg BDNF.

7. The method of claim 1, wherein the composition comprises from 0.05 mg to 0.20 mg BDNF.

8. The method of claim 7, wherein the composition comprises about 0.10 mg BDNF.

9. The method of claim 1, wherein the composition comprises from 0.20 mg to 0.40 mg BDNF.

10. The method of claim 9, wherein the composition comprises about 0.3 mg BDNF.

11. The method of claim 1, wherein the composition comprises from 0.40 mg to 1.10 mg BDNF.

12. The method of claim 9, wherein the composition comprises about 0.75 mg or about 0.78 mg BDNF.

13. The method of claim 1, wherein the composition comprises from 1.10 mg to 1.90 mg BDNF.

14. The method of claim 9, wherein the composition comprises about 1.5 mg or about 1.56 mg BDNF.

15. The method of claim 1, wherein the auris-acceptable vehicle is an auris-acceptable gel.

16. The method of claim 15, wherein the auris-acceptable gel is a thermoreversible gel.

17. The method of claim 16, wherein the auris-acceptable gel comprises a copolymer of polyoxyethylene and polyoxypropylene.

18. The method of claim 17, wherein the copolymer of polyoxyethylene and polyoxypropylene is poloxamer 407.

19. The method of claim 18, wherein the otic formulation comprises from about 14 wt % to about 18 wt % poloxamer 407.

20. The method of claim 18, wherein the otic formulation comprises from about 15 wt % to about 17 wt % poloxamer 407.

21. The method of claim 18, wherein the otic formulation comprises about 15.8 wt % or about 16 wt % poloxamer 407.

22. The method of claim 16, wherein the auris-acceptable gel has a gelation viscosity from about 15,000 cP and about 3,000,000 cP.

23. The method of claim 16, wherein the auris-acceptable gel is capable of being injected by a narrow gauge needle or cannula through the tympaniC membrane.

24. The method of claim 16, wherein the otic formulation has an osmolarity from about 100 mOsm/L to about 1000 mOsm/L.

25. The method of claim 16, wherein the otic formulation has a gelation temperature from about 19° C. to about 42° C.

26. The method of claim 16, wherein the otic formulation has a pH from about 7.0 to about 8.0.

27. The method of claim 1, wherein the otic formulation is an aqueous formulation and essentially free of any non-aqueous solvents.

28. The method of claim 1, wherein the growth factor is dissolved in the otic formulation.

29. The method of claim 1, wherein the growth factor is suspended in the otic formulation.

30. The method of claim 1, wherein the otic formulation provides sustained release of BDNF into the inner ear over a period of at least 5 days.

31. The method of claim 1, wherein the otic formulation provides sustained release of BDNF into the inner ear over a period of at least 1 week.

32. The method of claim 1, wherein the otic formulation provides sustained release of BDNF into the inner ear over a period of at least 2 weeks.

33. The method of claim 1, wherein the otic formulation provides sustained release of BDNF into the inner ear over a period of at least 3 weeks.

34. The method of claim 1, wherein the otic formulation provides sustained release of BDNF into the inner ear over a period of at least 4 weeks.

35. The method of claim 1, wherein the otic formulation repairs ribbon synapses.

36. The method of claim 1, wherein the hearing loss or hearing impairement is selected from cochlear synaptopathy, hearing-in-noise difficulties, speech-in-noise hearing impairement, or combinations thereof.

37. The method of claim 1, wherein the hearing loss or hearing impairement is cochlear synaptopathy.

38. The method of claim 1, wherein the hearing loss or hearing impairement is hearing-in-noise difficulties.

39. The method of claim 1, wherein the hearing loss or hearing impairement is speech-in-noise hearing impairement.
